(19) United States
(12) Patent Application Publication
(10) Pub. No.: US 2024/0317872 A1
(43) Pub. Date: Sep. 26, 2024

(54) POLYPEPTIDE COMPLEXES WITH IMPROVED STABILITY AND EXPRESSION

(71) Applicant: WUXI BIOLOGICS IRELAND LIMITED, DUNDALK, CO LOUTH (IE)

(72) Inventors: Jianqing XU, Shanghai (CN); Shuang WANG, Shanghai (CN); Jijie GU, Shanghai (CN)

(21) Appl. No.: 18/272,548

(22) PCT Filed: Jan. 18, 2022
(86) PCT No.: PCT/CN2022/072592

§ 371 Date: Jul. 14, 2023

(30) Foreign Application Priority Data
Jan. 19, 2021 (WO) PCT/CN2021/072601

(51) Int. Cl. C07K 16/28; A61K 38/00; A61K 39/00; C07K 14/725; C07K 16/24; C07K 16/32

(52) U.S. Cl. CPC C07K 16/2878; C07K 14/7051; C07K 16/244; C07K 16/2827; C07K 16/32; A61K 38/00; A61K 2039/505; C07K 2317/31; C07K 2317/75; C07K 2317/92; C07K 2317/94; C07K 2319/30

(57) ABSTRACT

A polypeptide complex is provided. The polypeptide complex comprises antibody variable regions of the heavy chain and light chain respectively fused to modified TCR constant regions, wherein the modified TCR constant regions comprises at least one mutations to stabilize the polypeptide complex such that polypeptide complex has improved stability and/or expression level. A bispecific antigen binding polypeptide complex that contains a first antigen-binding moiety of the polypeptide complex and a second antigen-binding moiety, methods of producing the polypeptide complex or the bispecific antigen binding polypeptide complex, methods of treating disease or disorder using the polypeptide complex or the bispecific antigen binding polypeptide complex, polypeptides encoding the polypeptide complex and/or the bispecific antigen binding polypeptide complex, vectors and host cells containing the polypeptides, compositions and pharmaceutical compositions comprising the polypeptide complex and/or the bispecific antigen binding polypeptide complex are also provided herein.

### CROSS-REFERENCING

This application claims the benefit of International application PCT/CN2021/072601, filed on Jan. 19, 2021, which is incorporated by reference in its entirety.

### FIELD OF THE INVENTION

The present disclosure generally relates to polypeptide complexes comprising antibody variable regions fused to modified TCR constant regions, and multispecific polypeptide complexes comprising the same, and methods of preparation and use thereof.

### BACKGROUND

Multispecific such as bispecific antibodies are growing to be a new category of therapeutic reagents. The unique capability of bispecific antibodies in binding two different targets or two different epitopes enabled many new mechanisms of action (MOA) that are not accessible by regular antibodies. In order to apply these new MOA into clinical development, we developed a bispecific platform named WuXiBody™, which can assemble two parental antibodies into a bispecific molecule with desired valency and functionality, as disclosed in PCT/CN2018/106766 (WO 2019/057122), the full content of which is herein incorporated by reference.

For example, as disclosed in PCT/CN2018/106766 (WO 2019/057122), the WuXiBody™ platform employed certain TCR constant regions (CBeta/CAlpha) to replace the CH1 and CL domains of an antibody Fab. The chimeric Fab possesses a unique light-heavy chain interface that is orthogonal to that of a regular antibody Fab. The assembly of the chimeric and regular Fabs in different formats can create various bispecific molecules with different structures and valences.

There is a great need to design multispecific, e.g., bispecific, molecules with desirable expression level, stability, and/or affinity to antigens. The present disclosure describes certain multispecific such as bispecific polypeptide complexes with desired stability and/or protein expression levels through engineering, e.g., the CAlpha and/or CBeta c-terminus regions and/or the residues that are spatially close to the c-terminus of CAlpha and/or CBeta.

### SUMMARY OF THE DISCLOSURE

In one aspect, the present disclosure provides a polypeptide complex comprising a first polypeptide comprising, from N-terminus to C-terminus, a first heavy chain variable domain (VH) of a first antibody operably linked to a first T cell receptor (TCR) constant region (C1), and a second polypeptide comprising, from N-terminus to C-terminus, a first light chain variable domain (VL) of the first antibody operably linked to a second TCR constant region (C2), wherein C1 and C2 are capable of forming a dimer comprising at least one non-native interchain bond between C1 and C2, and the non-native interchain bond is capable of stabilizing the dimer, and the first antibody has a first antigenic specificity, wherein C1 comprises an engineered CBeta, and C2 comprises an engineered CAlpha, or C1 comprises an engineered CAlpha, and C2 comprises an engineered CBeta.

In one aspect, the present disclosure provides a bispecific polypeptide complex, comprising a first antigen-binding moiety associated with a second antigen-binding moiety, wherein the first antigen-binding moiety comprises a first polypeptide comprising, from N-terminal to C-terminal, a first heavy chain variable domain (VH) of a first antibody operably linked to a first T cell receptor (TCR) constant region (C1), and a second polypeptide comprises, from N-terminal to C-terminal, a first light chain variable domain (VL) of the first antibody operably linked to a second TCR constant region (C2), wherein C1 and C2 are capable of forming a dimer comprising at least one non-native interchain bond between a first mutated residue comprised in C1 and a second mutated residue comprised in C2, and the non-native interchain bond is capable of stabilizing the dimer, wherein C1 comprises an engineered CBeta, and C2 comprises an engineered CAlpha; or C1 comprises an engineered CAlpha, and C2 comprises an engineered CBeta, and wherein the first antibody has a first antigenic specificity, a second antigen-binding moiety has a second antigenic specificity which is different from the first antigenic specificity.

In certain embodiments, the polypeptide complex disclosed herein comprises at least one mutation at the CAlpha and/or CBeta c-terminus regions or the residues that are spatially close to the c-terminus of CAlpha and/or CBeta. In some embodiments, the at least one mutation improves the stability of CAlpha, the stability of CBeta, and/or the CAlpha-CBeta interfacial stability compared to the CAlpha and/or CBeta without such mutation. In some embodiments, the polypeptide complex disclosed with the at least one mutation has improved expression level, stability, and/or affinity to antigen(s) compared to the polypeptide complex without such mutation.

In one aspect, the present disclosure provides a bispecific fragment of the bispecific polypeptide complex provided herein. In one aspect, the present disclosure provides herein a conjugate comprising the polypeptide complex provided herein, or the bispecific polypeptide complex provided herein, conjugated to a moiety.

In one aspect, the present disclosure provides herein an isolated polynucleotide encoding the polypeptide complex provided herein, or the bispecific polypeptide complex provided herein. In one aspect, the present disclosure provides herein an isolated vector comprising the polynucleotide provided herein. In one aspect, the present disclosure provides herein a host cell comprising the isolated polynucleotide provided herein or the isolated vector provided herein. In one aspect, the present disclosure provides herein a method of expressing the polypeptide complex provided herein, or the bispecific polypeptide complex provided herein, comprising culturing the host cell provided herein under the condition at which the polypeptide complex, or the bispecific polypeptide complex is expressed.

In one aspect, the present disclosure provides herein a method of producing the polypeptide complex provided herein, comprising a) introducing to a host cell a first polynucleotide encoding a first polypeptide comprising, from N-terminal to C-terminal, a first heavy chain variable domain (VH) of a first antibody operably linked to a first TCR constant domain (C1), and a second polynucleotide encoding a second polypeptide comprising, from N-terminal to C-terminal, a first light chain variable domain (VL) of the first antibody operably linked to a second TCR constant domain (C2), wherein C1 and C2 are capable of forming a dimer comprising at least one non-native interchain bond between C1 and C2, and the non-native interchain bond is capable of stabilizing the dimer of C1 and C2, and the first antibody has a first antigenic specificity; b) allowing the host cell to express the polypeptide complex.

In one aspect, the present disclosure provides herein a method of producing a bispecific polypeptide complex provided herein, comprising a) introducing to a host cell a first polynucleotide encoding a first polypeptide comprising, from N-terminal to C-terminal, a first heavy chain variable domain (VH) of a first antibody operably linked to a first TCR constant region (C1), a second polynucleotide encoding a second polypeptide comprising, from N-terminal to C-terminal, a first light chain variable domain (VL) of the first antibody operably linked to a second TCR constant region (C2), a third polynucleotide encoding a third polypeptide comprising VH of a second antibody, and a fourth polynucleotide encoding a fourth polypeptide comprising VL of the second antibody, wherein C1 and C2 are capable of forming a dimer comprising at least one non-native interchain bond between C1 and C2, and the non-native interchain bond is capable of stabilizing the dimer, and the first antibody has a first antigenic specificity and the second antibody has a second antigenic specificity; b) allowing the host cell to express the bispecific polypeptide complex.

In one aspect, the present disclosure provides herein a method of producing a bispecific polypeptide complex provided herein, comprising a) introducing to a host cell a first polynucleotide encoding a first polypeptide comprising, from N-terminal to C-terminal, a first heavy chain variable domain (VH) and constant region (CH1) of a first antibody operably linked to a second heavy chain variable domain (VH) of a second antibody operably linked to a first TCR constant region (C1), a second polynucleotide encoding a second polypeptide comprising, from N-terminal to C-terminal, a second light chain variable domain (VL) of the second antibody operably linked to a second TCR constant region (C2), a third polynucleotide encoding a third polypeptide comprising VL of the first antibody, wherein C1 and C2 are capable of forming a dimer comprising at least one non-native interchain bond between C1 and C2, and the non-native interchain bond is capable of stabilizing the dimer, and the first antibody has a first antigenic specificity and the second antibody has a second antigenic specificity; b) allowing the host cell to express the bispecific polypeptide complex. In certain embodiments, the method of producing the bispecific polypeptide complex provided herein further comprising isolating the polypeptide complex.

In certain embodiments, the methods of producing the bispecific polypeptide complex provided herein further comprises isolating the polypeptide complex.

In one aspect, the present disclosure provides a composition comprising the polypeptide complex provided herein, or the bispecific polypeptide complex provided herein. In one aspect, the present disclosure provides herein a pharmaceutical composition comprising the polypeptide complex provided herein, or the bispecific polypeptide complex provided herein and a pharmaceutically acceptable carrier.

In one aspect, the present disclosure provides herein a method of treating a condition in a subject in need thereof, comprising administrating to the subject a therapeutically effective amount of the polypeptide complex provided herein, or the bispecific polypeptide complex provided herein. In certain embodiments, the condition can be alleviated, eliminated, treated, or prevented when the first antigen and the second antigen are both modulated.

In certain embodiments, the non-native interchain bond is formed between a first mutated residue comprised in C1 and a second mutated residue comprised in C2. In certain embodiments, at least one of the first and the second mutated residues is a cysteine residue. In certain embodiments, the non-native interchain bond is a disulphide bond. In certain embodiments, the first mutated residue is comprised within a contact interface of C1, and/or the second mutated residue is comprised within a contact interface of C2. In certain embodiments, at least one native cysteine residue is absent or present in C1 and/or C2. In certain embodiments, the native cysteine residue at position C74 of engineered CBeta is absent or present. In certain embodiments, the native C74 is absent in CBeta. In certain embodiments, the native disulfide bond (C96 on CAlpha, and A128, D129 and C130 on CBeta) may be present or absent.

In certain embodiments, at least one native N-glycosylation site is absent or present in C1 and/or C2. In certain embodiments, the native N-glycosylation sites are absent in C1 and/or C2.

In certain embodiments, the dimer as disclosed herein comprises 1, 2, 3, 4, 5, 6, 7, 8, 9, 10, 11, 12, 13, 14, 15 or more non-native interchain bonds. In certain embodiments, at least one of the non-native interchain bonds is disulphide bond. In certain embodiments, the dimer comprises 1, 2, 3, 4, 5, 6, 7, 8, 9, 10, 11, 12, 13, 14, 15 or more disulphide bonds.

In certain embodiments, the first VH is operably linked to C1 at a first conjunction domain, and the first VL is operably linked to C2 at a second conjunction domain. In certain embodiments, the first VH associates to C1 at a first conjunction domain via a connector, the first VL associates to C2 at a second conjunction domain via a connector.

In certain embodiments, the first and/or the second conjunction domain comprises a proper length (e.g., 0, 1, 2, 3, 4, 5, 6, 7, 8, 9, or 10 amino acid residues) of the C terminal fragment of antibody V/C conjunction, and a proper length (e.g., 0, 1, 2, 3, 4, 5, 6, 7, 8, 9, or 10 amino acid residues) of the N terminal fragment of TCR V/C conjunction.

In certain embodiments, the engineered CBeta comprises a mutated residue such as a mutated cysteine residue within a contact interface selected from the group consisting of amino acid residues 9-35, 52-66, 71-86, and 122-127; and/or the engineered CAlpha comprises a mutated residue such as a mutated cysteine residue within a contact interface selected from a group consisting of amino acid residues 6-29, 37-67, and 86-95.

In certain embodiments, the engineered CBeta comprises a mutated residue K9E.

In certain embodiments, the engineered CBeta comprises a mutated cysteine residue that substitutes for an amino acid residue at a position selected from: S56C, S16C, F13C, V12C, E14C, L62C, D58C, S76C, and R78C, and/or the engineered CAlpha comprises a mutated cysteine residue that substitutes for an amino acid residue at a position selected from: T49C, Y11C, L13C, S16C, V23C, Y44C, T46C, L51C, and S62C.

In certain embodiments, the engineered CBeta and the engineered CAlpha comprise a pair of mutated cysteine residues that substitute for a pair of amino acid residues selected from the group consisting of: S16C in CBeta and Y11C in CAlpha, F13C in CBeta and L13C in CAlpha, S16C in CBeta and L13C in CAlpha, V12C in CBeta and S16C in CAlpha, E14C in CBeta and S16C in CAlpha, F13C in CBeta and V23C in CAlpha, L62C in CBeta and Y44C in CAlpha, D58C in CBeta and T46C in CAlpha, S76C in CBeta and T46C in CAlpha, S56C in CBeta and T49C in CAlpha, S56C in CBeta and L51C in CAlpha, S56C in CBeta and S62C in CAlpha, and R78C in CBeta and S62C in CAlpha, and wherein the pair of cysteine residues are capable of forming a non-native interchain disulphide bond.

In certain embodiments, at least one native glycosylation site is absent or present in the engineered CBeta and/or in the engineered CAlpha. In certain embodiments, the native glycosylation site in the engineered CBeta is N69, and/or the native glycosylation site(s) in the engineered CAlpha is/are selected from N34, N68, N79, and any combination thereof.

In certain embodiments, the engineered CBeta lacks or retains an FG loop encompassing amino acid residues 101-117 of the native CBeta and/or a DE loop encompassing amino acid residues 66-71 of the native CBeta.

In certain embodiments, the first polypeptide further comprises an antibody CH2 domain, and/or an antibody CH3 domain.

In certain embodiments, the first antigenic specificity and the second antigenic specificity are directed to two different antigens, or are directed to two different epitopes on one antigen.

In certain embodiments, the first antigen-binding moiety binds to CTLA-4. In certain embodiments, the second antigen-binding moiety binds to PD-L1. In certain embodiments, the first antigen-binding moiety binds to PD-L1. In certain embodiments, the second antigen-binding moiety binds to CTLA-4. In certain embodiments, the first antigen-binding moiety binds to PD-L1. In certain embodiments, the second antigen-binding moiety binds to 4-1BB. In certain embodiments, the first antigen-binding moiety binds to 4-1BB. In certain embodiments, the second antigen-binding moiety binds to PD-L1. In certain embodiments, the first and second antigen-binding moieties binds to two separate domains of HER2, respectively. In certain embodiments, the first antigen-binding moiety binds to HER2 D2 In certain embodiments, the second antigen-binding moiety binds to HER2 D4. In certain embodiments, the first antigen-binding moiety binds to HER2 D4. In certain embodiments, the second antigen-binding moiety binds to HER2 D2. In certain embodiments, the first antigen-binding moiety binds to IL-17. In certain embodiments, the second antigen-binding moiety binds to IL-20. In certain embodiments, the first antigen-binding moiety binds to IL-20. In certain embodiments, the second antigen-binding moiety binds to IL-17. In certain embodiments, the first antigen-binding moiety binds to IL-4. In certain embodiments, the second antigen-binding moiety binds to IL-13. In certain embodiments, the first antigen-binding moiety binds to IL-13. In certain embodiments, the second antigen-binding moiety binds to IL-4.

In certain embodiments, the association of the polypeptide complex disclosed herein is via a connecter, a disulphide bond, a hydrogen bond, electrostatic interaction, a salt bridge, or hydrophobic-hydrophilic interaction, or the combination thereof.

In certain embodiments, the second antigen-binding moiety comprises a heavy chain variable domain and a light chain variable domain of a second antibody having the second antigenic specificity. In certain embodiments, the second antigen-binding moiety comprises a Fab. In certain embodiments, the first antigenic specificity and the second antigenic specificity are directed to two different antigens, or are directed to two different epitopes on one antigen.

In certain embodiments, one of the first and the second antigenic specificities is directed to a T-cell specific receptor molecule and/or a natural killer cell (NK cell) specific receptor molecule, and the other is directed to a tumor associated antigen. In certain embodiments, one of the first and the second antigenic specificities is directed to CD3, and the other is directed to a tumor associated antigen. In certain embodiments, one of the first and the second antigenic specificities is directed to CD3, and the other is directed to CD19.

In certain embodiments, the first antigen-binding moiety further comprises a first dimerization domain, and the second antigen-binding moiety further comprises a second dimerization domain, wherein the first and the second dimerization domains are associated.

In certain embodiments, the association of the first and second dimerization domains is via a connecter, a disulphide bond, a hydrogen bond, electrostatic interaction, a salt bridge, or hydrophobic-hydrophilic interaction, or the combination thereof.

In certain embodiments, the first and/or the second dimerization domain comprises at least a portion of an antibody hinge region, optionally derived from IgG1, IgG2 or IgG4.

In certain embodiments, the first and/or the second dimerization domain further comprises at least a portion of an antibody hinge region, an antibody CH2 domain, and/or an antibody CH3 domain. In certain embodiments, the first dimerization domain is operably linked to the first TCR constant region (C1) at a third conjunction domain.

In certain embodiments, the second dimerization domain is operably linked to the heavy chain variable domain of the second antigen-binding moiety.

In certain embodiments, the first and the second dimerization domains are different and associate in a way that discourages homodimerization and/or favors heterodimerization.

In certain embodiments, the first and the second dimerization domains are capable of associating into heterodimers via knobs-into-holes, hydrophobic interaction, electrostatic interaction, hydrophilic interaction, or increased flexibility.

In certain embodiments, the engineered CAlpha and CBeta comprise at least one residue substituted with at least one corresponding amino acid residue that contributes to the stability of murine TCR, to improve the stability and/or expression level of the polypeptide complex disclosed herein.

In certain embodiments, the engineered CAlpha comprises one or more mutated residues, e.g., 1, 2, 3, 4, 5, or 6 mutated residues, at the C-terminus, wherein mutated residues comprise a fragment derived from a human IgG1 hinge sequence, a human kappa light c-terminal sequence, a PDB-5DK3 (human IgG4) CH1 region sequence, a human lambda light chain c-terminus region sequence, a PDB-1IGT (mouse IgG2a) CH1 region sequence, a PDB-1IGT (mouse IgG2a) kappa light chain c-terminus region sequence, a human IgG2 hinge region sequence (e.g., ERKCC-ERKSC, SEQ ID NO: 3), PDB-5E8E (human IgA) CH1 region sequence, a PDB-5E8E (human IgA) kappa light chain c-terminus region sequence, a PDB-1DEE (human IgM) CH1 region sequence, a human IgE CH1 region sequence, or a human IgD CH1 region sequence.

In certain embodiments, one or more modifications of CAlpha, for example at its C-terminus, may improve the stability and/or expression level of the polypeptide complex disclosed herein. In certain embodiments, replacing the CAlpha c-terminus residues (residues at the c-terminal region of CAlpha, e.g., amino acid residues at positions 81-96, 84-95, 92-95, or 92-96) by a human IgG1 sequence segment or human kappa light c-terminal segment may improve the stability and/or expression level of the polypeptide complex disclosed herein. In certain embodiments, the engineered CAlpha comprises mutations at the C-terminus of CAlpha wherein the C-terminus comprises an amino acid sequence, e.g., “EPKS” (SEQ ID NO: 4) and “VEPKS” (SEQ ID NO: 5), derived from a human IgG1 hinge sequence. For example, the engineered CAlpha comprises mutations at the C-terminus of CAlpha with “VEPKS” (SEQ ID NO: 5) in place of “PESS” (SEQ ID NO: 6). For another example, the engineered CAlpha comprises mutations at the C-terminus of CAlpha with “EPKS” (SEQ ID NO: 4) in place of “PESS” (SEQ ID NO: 6). In certain embodiments, the engineered CAlpha comprises mutations at the C-terminus of CAlpha wherein the C-terminus comprises an amino acid sequence, e.g., “NRGE” (SEQ ID NO: 7), derived from human kappa light c-terminal residues. For example, the engineered CAlpha comprises mutations at the C-terminus of CAlpha with “NRGE” (SEQ ID NO: 7) in place of “PESS” (SEQ ID NO: 6).

In certain embodiments, modifications of at least one amino acid residue on CBeta, for example, the residues that are structurally or spatially close to the C-terminus of CAlpha, may also improve the stability and/or expression level of the polypeptide complexes disclosed herein.

In certain embodiments, the engineered CAlpha and/or CBeta comprise one or more residues mutated to corresponding one or more amino acid residues from murine TCR to improve stability and/or expression levels of the polypeptide complexes disclosed herein. For example, the engineered CAlpha comprises at least one mutated residue selected from substitutions P92S, E93D, S94V, and S95P, and/or the engineered CBeta comprises at least one mutated residue selected from substitutions E17K and S21A. In certain embodiments, the engineered CAlpha comprises mutated residues P92S, E93D, S94V, and S95P. In certain embodiments, the engineered CBeta comprises mutated residues E17K and S21A. In certain embodiments, the engineered CAlpha comprises mutated residues P92S, E93D, S94V, and S95P, and the engineered CBeta comprises mutated residues E17K and S21A.

In certain embodiments, the engineered CAlpha and/or the engineered CBeta comprise one, two, three, four, five, six, or seven mutations chosen from S22F, T33L, and A73T on CAlpha and E17K, H22R, D38P, and S53D on CBeta.

In certain embodiments, at least one native cysteine residue can be absent or present in the engineered CAlpha and CBeta. In certain embodiments, the native residues A128, D129 and C130 are absent in CBeta and/or the native residue C96 is absent in CAlpha. In certain embodiments, the native residues A128, D129, and C130 are present in CBeta and/or the native residue C96 is present in CAlpha, such that the original TCR native disulfide bond formed by the two Cys residues is present.

In certain embodiments, the engineered CAlpha and/or CBeta comprise one or more mutated residues to form one or more non-native disulfide bonds, selected from: P8C on CAlpha, A9C on CAlpha, V10C on CAlpha, F26C on CAlpha, F29C on CAlpha, T33C on CAlpha, Q34C on CAlpha, V35C on CAlpha, S36C on CAlpha, S38C on CAlpha, K39C on CAlpha, F78C on CAlpha, N80C on CAlpha, S81C on CAlpha, I82C on CAlpha, P84C on CAlpha, D86C on CAlpha, T87C on CAlpha, F88C on CAlpha, F89C on CAlpha, P90C on CAlpha, and A18C on CBeta.

In certain embodiments, the engineered CAlpha and/or CBeta comprise one or more groups of mutated residues to form non-native disulfide bonds, selected from: F26C and F78C on CAlpha, S36C and N80C on CAlpha, S38C and N80C on CAlpha, K39C and N80C on CAlpha, Q34C and S81C on CAlpha, V35C and S81C on CAlpha, S36C and S81C on CAlpha, Q34C and I82C on CAlpha, P8C and P84C on CAlpha, F29C and P84C on CAlpha, T33C and P84C on CAlpha, P8C and D86C on CAlpha, P8C and T87C on CAlpha, A9C and F88C on CAlpha, V10C and F89C on CAlpha, P90C on CAlpha and A18C on CBeta, P8C, D86C, P90C on CAlpha and A18C on CBeta, P8C and D86C on CAlpha.

In certain embodiments, the engineered CAlpha comprises mutated residues: F26C and F78C on CAlpha.

In certain embodiments, the engineered CAlpha comprises mutated residues: S36C and N80C on CAlpha.

In certain embodiments, the engineered CAlpha comprises mutated residues: S38C and N80C on CAlpha.

In certain embodiments, the engineered CAlpha comprises mutated residues: K39C and N80C on CAlpha.

In certain embodiments, the engineered CAlpha comprises mutated residues: Q34C and S81C on CAlpha.

In certain embodiments, the engineered CAlpha comprises mutated residues: V35C and S81C on CAlpha.

In certain embodiments, the engineered CAlpha comprises mutated residues: S36C and S81C on CAlpha.

In certain embodiments, the engineered CAlpha comprises mutated residues: Q34C and 182C on CAlpha.

In certain embodiments, the engineered CAlpha comprises mutated residues: P8C and P84C on CAlpha.

In certain embodiments, the engineered CAlpha comprises mutated residues: F29C and P84C on CAlpha.

In certain embodiments, the engineered CAlpha comprises mutated residues: T33C and P84C on CAlpha.

In certain embodiments, the engineered CAlpha comprises mutated residues: P8C and D86C on CAlpha.

In certain embodiments, the engineered CAlpha comprises mutated residues: P8C and T87C on CAlpha.

In certain embodiments, the engineered CAlpha comprises mutated residues: A9C and F88C on CAlpha.

In certain embodiments, the engineered CAlpha comprises mutated residues: V10C and F89C on CAlpha.

In certain embodiments, the engineered CBeta comprises a mutated residue: A18C on CBeta.

In certain embodiments, the engineered CAlpha and the engineered CBeta comprise mutated residues: P90C on CAlpha and A18C on CBeta.

In certain embodiments, the engineered CAlpha and the engineered CBeta comprise mutated residues: P8C, D86C, and P90C on CAlpha, and A18C on CBeta.

In certain embodiments, the engineered CAlpha comprises mutated residues: P8C and D86C on CAlpha.

In certain embodiments, the engineered CBeta comprises a Gly amino acid residue at the C-terminal end of CBeta such that the Gly amino acid residue is adjacent to or forms part of the hinge region.

In certain embodiments, the engineered CAlpha comprises a deletion of 1 to 22 amino acid residues, e.g., 1, 2, 3, 4, 5, 6, 7, 8, 9, 10, 11, 12, 13, 14, 15, 16, 17, 18, 19, 20, 21, or 22, at or around its C-terminus. In certain embodiments, the engineered CAlpha comprises a deletion of 8 amino acid residues (e.g., amino acid residues 88-95) at or around its C-terminus, such as amino acid residues “FFPSPESS” (SEQ ID NO: 9). In certain embodiments, the engineered CAlpha comprises a deletion of 4 amino acid residues (e.g., amino acid residues 92-95) at or around its C-terminus, such as amino acid residues “PESS” (SEQ ID NO: 6).

In certain embodiments, the engineered CAlpha and CBeta comprise amino acid residue glycine at the C-terminal end of CBeta, and mutations at the C-terminus of CAlpha with “VEPKS” (SEQ ID NO: 5) in place of “PESS” (SEQ ID NO: 6).

In certain embodiments, the engineered CAlpha and CBeta comprise the original TCR native disulfide bond (C96 on CAlpha, and A128, D129 and C130 on CBeta), amino acid residue glycine at the C-terminal end of CBeta, and mutations at the C-terminus of CAlpha with “NRGE” (SEQ ID NO: 7) in place of “PESS” (SEQ ID NO: 6).

In certain embodiments, the engineered CAlpha and CBeta comprise the original TCR native disulfide bond (C96 on CAlpha, and A128, D129 and C130 on CBeta), and mutations P92S, E93D, S94V, and S95P at the C-terminus of CAlpha, E17K and S21A on CBeta.

In certain embodiments, the engineered CAlpha and CBeta comprise the original TCR native disulfide bond (C96 on CAlpha, and A128, D129 and C130 on CBeta), amino acid residue glycine at the C-terminal end of CBeta, and mutations at the C-terminus of CAlpha with “VEPKS” (SEQ ID NO: 5) in place of “PESS” (SEQ ID NO: 6).

In certain embodiments, the engineered CAlpha and CBeta comprise the original TCR native disulfide bond (C96 on CAlpha, and A128, D129 and C130 on CBeta), amino acid residue glycine at the C-terminal end of CBeta, and mutations at the C-terminus of CAlpha with “NRGE” (SEQ ID NO: 7) in place of “PESS” (SEQ ID NO: 6).

In certain embodiments, the engineered CAlpha comprises mutated residues F26C and F78C on Calpha, and a deletion of 4 amino acid residues at Calpha C terminal (amino acid residues 92-95).

In certain embodiments, the engineered CAlpha comprises mutated residues S36C and N80C on Calpha, and a deletion of 4 amino acid residues at Calpha C terminal (amino acid residues 92-95).

In certain embodiments, the engineered CAlpha comprises mutated residues S38C and N80C on Calpha and a deletion of 4 amino acid residues at Calpha C terminal (amino acid residues 92-95).

In certain embodiments, the engineered CAlpha comprises mutated residues K39C and N80C on Calpha, and a deletion of 4 amino acid residues at Calpha C terminal (amino acid residues 92-95).

In certain embodiments, the engineered CAlpha comprises mutated residues Q34C and S81C on Calpha, and a deletion of 4 amino acid residues at Calpha C terminal (amino acid residues 92-95).

In certain embodiments, the engineered CAlpha comprises mutated residues V35C and S81C on Calpha, and a deletion of 4 amino acid residues at Calpha C terminal (amino acid residues 92-95).

In certain embodiments, the engineered CAlpha comprises mutated residues S36C and S81C on Calpha, and a deletion of 4 amino acid residues at Calpha C terminal (amino acid residues 92-95).

In certain embodiments, the engineered CAlpha comprises mutated residues Q34C and I82C on Calpha, and a deletion of 4 amino acid residues at Calpha C terminal (amino acid residues 92-95).

In certain embodiments, the engineered CAlpha comprises mutated residues P8C and P84C on Calpha, and a deletion of 4 amino acid residues at Calpha C terminal (amino acid residues 92-95).

In certain embodiments, the engineered CAlpha comprises mutated residues F29C and P84C on Calpha, and a deletion of 4 amino acid residues at Calpha C terminal (amino acid residues 92-95).

In certain embodiments, the engineered CAlpha comprises mutated residues T33C and P84C on Calpha, and a deletion of 4 amino acid residues at Calpha C terminal (amino acid residues 92-95).

In certain embodiments, the engineered CAlpha comprises mutated residues P8C and D86C on Calpha, and a deletion of 4 amino acid residues at Calpha C terminal (amino acid residues 92-95).

In certain embodiments, the engineered CAlpha comprises mutated residues P8C and T87C on Calpha, and a deletion of 4 amino acid residues at Calpha C terminal (amino acid residues 92-95).

In certain embodiments, the engineered CAlpha comprises mutated residues A9C and F88C on Calpha, and a deletion of 4 amino acid residues at Calpha C terminal (amino acid residues 92-95).

In certain embodiments, the engineered CAlpha comprises mutated residues V10C and F89C on Calpha, and a deletion of 4 amino acid residues at Calpha C terminal (amino acid residues 92-95).

In certain embodiments, the engineered CAlpha and the engineered CBeta comprise mutated residues with P90C on Calpha and A18C on Cbeta, and a deletion of 4 amino acid residues at Calpha C terminal (amino acid residues 92-95).

In certain embodiments, the engineered CAlpha and the engineered CBeta comprise mutations S22F, T33L and A73T on Calpha and E17K, H22R, D38P, and S53D on Cbeta.

In certain embodiments, the engineered CAlpha and the engineered CBeta comprise 7 mutations S22F, T33I, and A73T on Calpha, and E17K, H22R, D38P, and S53D on Cbeta, and the original TCR native disulfide bond (C96 on CAlpha and A128, D129 and C130 on CBeta).

In certain embodiments, the engineered CAlpha and the engineered CBeta comprise mutations P8C, D86C, and P90C on Calpha, and A18C on Cbeta.

In certain embodiments, the engineered CAlpha and the engineered CBeta comprise mutations P8C and D86C on Calpha, and comprise the original TCR native disulfide bond (C96 on CAlpha and A128, D129 and C130 on CBeta).

In certain embodiments, the engineered CAlpha and the engineered CBeta comprise mutations P90C on Calpha and A18C on Cbeta, and comprise the original TCR native disulfide bond (C96 on CAlpha and A128, D129 and C130 on CBeta).

In certain embodiments, the engineered CAlpha and the engineered CBeta comprise mutations P8C, D86C, and P90C on Calpha, and A18C on Cbeta, and comprise the original TCR native disulfide bond (C96 on CAlpha and A128, D129 and C130 on CBeta).

In certain embodiments, the engineered C2 comprises any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 79, and 80, and/or the engineered C1 comprises any one of SEQ ID NOs: 11, 13, 15, 17, 19, 21, 23, 25, 27, 29, 31, 33, 35, 37, 39, 41, 43, 45, 47, 49, 51, 53, 55, 57, 59, 61, 63, and 65. In certain embodiments, the engineered C1 comprises any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 79, and 80, and/or the engineered C2 comprises any one of SEQ ID NOs: 11, 13, 15, 17, 19, 21, 23, 25, 27, 29, 31, 33, 35, 37, 39, 41, 43, 45, 47, 49, 51, 53, 55, 57, 59, 61, 63, and 65. In certain embodiments, the engineered C1 and the engineered C2 respectively comprise a pair of sequences selected from the group consisting of SEQ ID NOs: 10/11, 12/13, 14/15, 16/17, 18/19, 20/21, 22/23, 24/25, 26/27, 28/29, 30/31, 32/33, 34/35, 36/37, 38/39, 40/41, 42/43, 44/45, 46/47, 48/49, 50/51, 52/53, 54/55, 56/57, 58/59, 60/61, 62/63, 64/65, 79/43, and 80/51. In certain embodiments, the engineered C2 and the engineered C1 respectively comprise a pair of sequences selected from the group consisting of SEQ ID NOs: 12/13, 14/15, 16/17, 18/19, 20/21, 22/23, 24/25, 26/27, 28/29, 30/31, 32/33, 34/35, 36/37, 38/39, 40/41, 42/43, 44/45, 46/47, 48/49, 50/51, 52/53, 54/55, 56/57, 58/59, 60/61, 62/63, 64/65, 79/43, and 80/51.

In certain embodiments, the engineered C2 and the engineered C1 respectively comprise a pair of sequences of SEQ ID NOs: 42 and 43. In certain embodiments, the engineered C1 and the engineered C2 respectively comprise a pair of sequences of SEQ ID NOs: 42 and 43.

In certain embodiments, the engineered C2 and the engineered C1 respectively comprise a pair of sequences of SEQ ID NOs: 50 and 51. In certain embodiments, the engineered C1 and the engineered C2 respectively comprise a pair of sequences of SEQ ID NOs: 50 and 51.

In certain embodiments, the engineered C2 and the engineered C1 respectively comprise a pair of sequences of SEQ ID NOs: 79 and 43. In certain embodiments, the engineered C1 and the engineered C2 respectively comprise a pair of sequences of SEQ ID NOs: 79 and 43.

In certain embodiments, the engineered C2 and the engineered C1 respectively comprise a pair of sequences of SEQ ID NOs: 80 and 51. In certain embodiments, the engineered C1 and the engineered C2 respectively comprise a pair of sequences of SEQ ID NOs: 80 and 51.

In certain embodiments, the polypeptide complex provided herein can be made into a Fab, a (Fab)₂, a bibody, a tribody, a triFabs, tandem linked Fabs, a Fab-Fv, tandem linked V domains, tandem linked scFvs, and among other formats.

In another aspect, the present disclosure provides a kit comprising the polypeptide complex provided herein for detection, diagnosis, prognosis, or treatment of a disease or condition.

The foregoing and other features and advantages of the disclosure will become more apparent from the following detailed description of several embodiments which proceeds with reference to the accompanying figures.

### BRIEF DESCRIPTION OF FIGURES

FIG. 1A shows SDS-PAGE characterizations of a first batch of T8311 proteins after purification, under non-reducing or reducing conditions. FIG. 1B shows SEC-HPLC characterizations of a first batch of T8311 proteins after purification.

FIGS. 2A and 2C shows SDS-PAGE characterizations of a second batch of T8311 proteins after purification, under non-reducing or reducing conditions. FIGS. 2B and 2C shows SEC-HPLC characterizations of a second batch of T8311 proteins after purification.

FIG. 3 shows DSF profiles of the first batch of T8311 proteins upon temperature increase.

FIG. 4 shows DSC curves of T8311 proteins, with those of T8311-57 and T8311-61 shifted to the right, indicating they had relatively stronger resistance to temperature increase, compared to T8311-1.

FIG. 5 shows DLS curves of T8311 proteins.

FIG. 6 shows mass spectra of T8311-1, T8311-57, and T8311-61.

FIG. 7 shows binding curves of T8311 proteins to target expression cells.

FIG. 8 shows reporter gene assay results to check the function of T8311 proteins. T8311-57 and T8311-61 showed good agonistic effects in activating 4-1BB mediated NF-KB pathway with PD-L1 expressing cells. G34 (T8311-U14T2.G34-1.uIgG1) showed weak agonist effect in the RGA test with PD-L1 expressing cells.

FIG. 9 shows PK analysis results of T8311 proteins. T8311-U14T2.G25R-57.uIgG1 and T8311-U14T2.G25R-61.uIgG1 showed longer t % with larger AUC and smaller clearance compared with other antibodies.

FIG. 10 shows SDS-PAGE (A) and SEC-HPLC (B) characterizations of W3618 proteins after purification.

FIG. 11 shows DSF profiles of W3618 proteins upon temperature increase.

FIG. 12 shows the sequences and numbering of TRAC_Human CAlpha (SEQ ID NO: 1) and an engineered CAlpha as disclosed herein, TRAC_WuXiBody 1.0 (SEQ ID NO: 10).

FIG. 13 shows the sequences and numbering of TRBC2_Human CBeta (SEQ ID NO: 2) and an engineered CBeta as disclosed herein, TRBC_WuXiBody 1.0 (SEQ ID NO: 8).

FIG. 14 illustrates two WuXiBody formats E17R and G25R, as well as a control format G34.

FIG. 15 shows PK analysis results of W3618 proteins. W3618-U4T1.E17R-57.uIgG1 and W3618-U4T1.E17R-61.uIgG1 showed longer t ½ with larger AUC and smaller clearance compared with W3618-U4T1.E17R-1.uIgG1.

FIG. 16 shows SDS-PAGE characterizations of W329001-U3T3 proteins after purification, under non-reducing or reducing conditions (upper panel), and SEC-HPLC characterizations of W329001-U3T3 proteins after purification (lower panel).

FIG. 17 shows DSF profiles of W329001-U3T3 proteins upon temperature increase.

FIG. 18 shows PK analysis results of W329001-U3T3 proteins. W329001-U3T3.G25R-57.uIgG1 and W329001-U3T3.G25R-61.uIgG1 showed longer t ½ with larger AUC and smaller clearance compared with W329001-U3T3.G25R-1.uIgG1.

FIG. 19 shows SDS-PAGE characterizations of W329001-U4T4 proteins after purification, under non-reducing or reducing conditions (top panel), and SEC-HPLC characterizations of W329001-U4T4 proteins after purification (middle and bottom panels).

FIG. 20 shows DSF profiles of W329001-U4T4 proteins upon temperature increase.

FIG. 21 shows PK analysis results of W329001-U4T4.G25R-57.uIgG1 and W329001-U4T4.G25R-61.uIgG1 proteins. W329001-U4T4.G25R-57.uIgG1 and W329001-U4T4.G25R-61.uIgG1 showed longer t ½ with larger AUC and smaller clearance compared with W329001-U4T4.G25R-1.uIgG1.

FIG. 22 shows a comparison of PK analysis results of W329001-U4T4.G25R-57.uIgG1 and W329001-U4T4.G25R-78.uIgG1 proteins.

FIG. 23 shows a comparison of PK analysis results of W329001-U4T4.G25R-61.uIgG1 and W329001-U4T4.G25R-79.uIgG1 proteins.

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety. If certain content of a reference cited herein contradicts or is inconsistent with the present disclosure, the present disclosure controls.

Certain terms as used herein are defined and/or explained in PCT/CN2018/106766 (WO 2019/057122). Those definitions and/or explanations are incorporated herein and apply to this disclosure unless stated otherwise.

### A. Polypeptide Complex

In one aspect, the present disclosure provides a polypeptide complex comprising a first polypeptide comprising, from N-terminus to C-terminus, a first heavy chain variable domain (VH) of a first antibody operably linked to a first T cell receptor (TCR) constant region (C1), and a second polypeptide comprising, from N-terminus to C-terminus, a first light chain variable domain (VL) of the first antibody operably linked to a second TCR constant region (C2), wherein C1 and C2 are capable of forming a dimer comprising at least one non-native interchain bond between C1 and C2, and the non-native interchain bond is capable of stabilizing the dimer, and the first antibody has a first antigenic specificity, wherein C1 comprises an engineered CBeta, and C2 comprises an engineered CAlpha, or C1 comprises an engineered CAlpha, and C2 comprises an engineered CBeta.

In one aspect, the present disclosure provides a bispecific polypeptide complex, comprising a first antigen-binding moiety associated with a second antigen-binding moiety, wherein the first antigen-binding moiety comprising a first polypeptide comprising, from N-terminal to C-terminal, a first heavy chain variable domain (VH) of a first antibody operably linked to a first T cell receptor (TCR) constant region (C1), and a second polypeptide comprising, from N-terminal to C-terminal, a first light chain variable domain (VL) of the first antibody operably linked to a second TCR constant region (C2), wherein C1 and C2 are capable of forming a dimer comprising at least one non-native interchain bond between a first mutated residue comprised in C1 and a second mutated residue comprised in C2, and the non-native interchain bond is capable of stabilizing the dimer, wherein C1 comprises an engineered CBeta, and C2 comprises an engineered CAlpha; or C1 comprises an engineered CAlpha, and C2 comprises an engineered CBeta, and wherein the first antibody has a first antigenic specificity, a second antigen-binding moiety has a second antigenic specificity which is different from the first antigenic specificity.

Depending on the multispecific format and/or numbering convenience, the numbering sequences as disclosed herein, such as first, second, and third, could be different. For example, the first VH may be numbered as the second VH, and the first VL may be numbered as the second VL. As another example, the second antigen-binding moiety may be numbered as the first antigen-binding moiety, and the first antigen-binding moiety may be numbered as the second antigen-binding moiety. As illustrated in FIG. 14, VH1 can be numbered as VH2, and VL1 can be numbered as VL2, etc., depending on preference and/or convenience.

In certain embodiments, the polypeptide complex disclosed herein comprises at least one mutation at the CAlpha and/or CBeta c-terminus regions or the residues that are spatially close to the c-terminus of CAlpha and/or CBeta. In some embodiments, the at least one mutation improves the stability of CAlpha, the stability of CBeta, and/or the CAlpha-CBeta interfacial stability compared to the CAlpha and/or CBeta without such mutation. In some embodiments, the polypeptide complex disclosed with the at least one mutation has improved expression level, stability, and/or affinity to antigens compared to the polypeptide complex without such mutation.

i. TCR Constant Region

The polypeptide complexes provided herein comprise constant regions derived from a TCR. Native TCR consists of two polypeptide chains, and has in general two types: one consists of alpha and beta chains (i.e. alpha/beta TCR), and the other consists of gamma and delta chains (i.e. gamma/delta TCR). These two types are structurally similar but have distinct locations and functions. About 95% human T cells have alpha/beta TCRs, whereas the rest 5% have gamma/delta TCRs. A precursor of alpha chain is also found and named as pre-alpha chain. Each of the two TCR polypeptide chains comprises an immunoglobulin domain and a membrane proximal region. The immunoglobulin region comprises a variable region and a constant region, and is characterized by the presence of an immunoglobulin-type fold. Each TCR polypeptide chain has a cysteine residue (e.g., at C terminal of the constant domain or at N terminal of the membrane proximal region) which together can form a disulphide bond that tethers the two TCR chains together.

Human TCR alpha chain constant region (referred to as CAlpha or Calpha) is known as TRAC, with the NCBI accession number of P01848, or an amino acid sequence of SEQ ID NO: 1, as shown in FIG. 12.

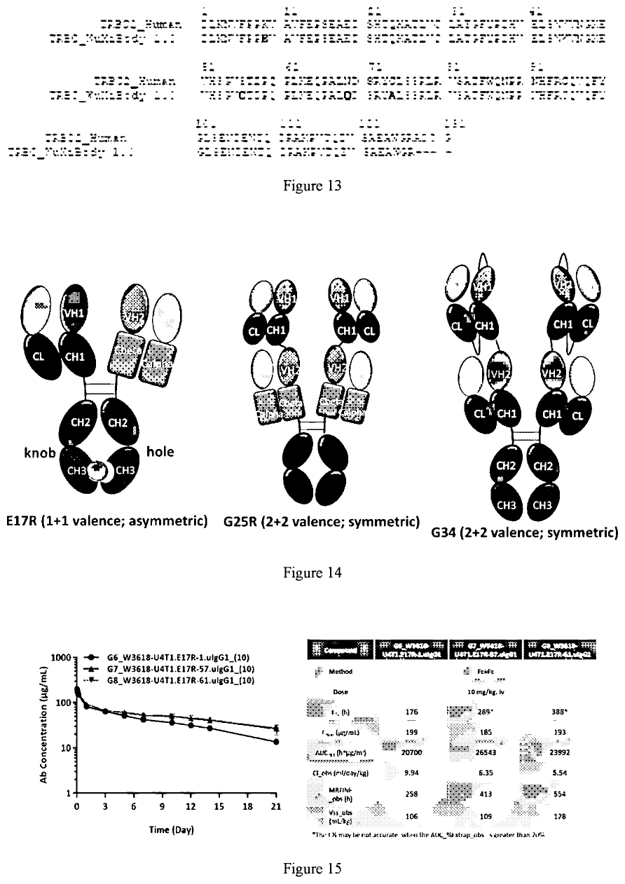

Human TCR beta chain constant region (referred to as CBeta or Cbeta) has two different variants, known as TRBC1 and TRBC2 (IMGT nomenclature) (see Toyonaga B, et al., PNAs, Vol. 82, pp. 8624-8628, Immunology (1985)). The amino acid sequence of TRBC2_Human (CBeta) is set forth in FIG. 13 as SEQ ID NO: 2.

Specifically, the native TCR beta chain contains a native cysteine residue at position 74, which is unpaired and therefore does not form a disulphide bond in a native alpha/beta TCR. In certain embodiments, in the polypeptide complexes provided herein, this native cysteine residue is absent or mutated to another residue. This may be useful to avoid incorrect intrachain or interchain pairing. In certain embodiments, the native cysteine residue is substituted for another residue, for example serine or alanine (e.g., C74A). In certain embodiments, the substitution can improve the TCR refolding efficiencies in vitro.

In the present disclosure, the first and the second TCR constant regions of the polypeptide complexes provided herein are capable of forming a dimer comprising, between the TCR constant regions, at least one non-native interchain bond that is capable of stabilizing the dimer. In some embodiments, the polypeptide complex disclosed herein comprises at least one mutation at the CAlpha and/or CBeta c-terminus regions or the residues that are spatially close to the c-terminus of CAlpha and/or CBeta to improve the stability of CAlpha, the stability of CBeta, and/or the CAlpha-CBeta interfacial stability.

An interchain bond is formed between one amino acid residue on one TCR constant region and another amino acid residue on the other TCR constant region. In certain embodiments, the non-native interchain bond can be any bond or interaction that is capable of associating two TCR constant regions into a dimer. Examples of suitable non-native interchain bond include, a disulphide bond, a hydrogen bond, electrostatic interaction, a salt bridge, or hydrophobic-hydrophilic interaction, a knobs-into-holes or the combination thereof. Examples of non-native interchain bonds are described in PCT/CN2018/106766 (WO 2019/057122) and are incorporated herein by reference.

In certain embodiments, the TCR constant region dimer comprises 1, 2, 3, 4, 5, 6, 7, 8, 9 or 10 non-native interchain bonds. Optionally, at least one of the 1, 2, 3, 4, 5, 6, 7, 8, 9 or 10 non-native interchain bonds are disulphide bonds, hydrogen bonds, electrostatic interaction, salt bridge, or hydrophobic-hydrophilic interaction, or any combination thereof.

The TCR constant region comprising a mutated residue is also referred to herein as an “engineered” TCR constant region, e.g., engineered CAlpha or engineered CBeta. In certain embodiments, the first TCR constant region (C1) of the polypeptide complex comprises an engineered TCR Alpha chain (CAlpha), and the second TCR constant region (C2) comprises an engineered TCR Beta chain (CBeta). In certain embodiments, C1 comprises an engineered CBeta, and C2 comprises an engineered CAlpha.

In certain embodiments, the engineered TCR constant region comprises one or more mutated cysteine residue. In certain embodiments, the one or more mutated residue is comprised within a contact interface of the first and/or the second engineered TCR constant regions.

The term “contact interface” as used herein refers to the particular region(s) on the polypeptides where the polypeptides interact/associate with each other. A contact interface comprises one or more amino acid residues that are capable of interacting with the corresponding amino acid residue(s) that comes into contact or association when interaction occurs. The amino acid residues in a contact interface may or may not be in a consecutive sequence. For example, when the interface is three-dimensional, the amino acid residues within the interface may be separated at different positions on the linear sequence.

In certain embodiments, the engineered CBeta comprises a mutated residue such as a mutated cysteine residue within a contact interface selected from the group consisting of: amino acid residues 9-35, 52-66, 71-86, and 122-127. In certain embodiments, the engineered CAlpha comprises a mutated residue such as a mutated cysteine residue within a contact interface selected from a group consisting of: amino acid residues 6-29, 37-67, and 86-95. Unless specified, the numbering of amino acid residues in the TCR constant region in the present disclosure is as set forth in FIGS. 12 and 13.

In certain embodiments, one or more disulphide bonds can be formed between the engineered CAlpha and the engineered CBeta. The mutated cysteine residue in CBeta can be a substitution selected from the group consisting of: S56C, S16C, F13C, V12C, E14C, F13C, L62C, D58C, S76C, and R78C, and/or the mutated cysteine residues in CAlpha can be a substitution selected from the group consisting of: T49C, Y11C, L13C, S16C, V23C, Y44C, T46C, L51C, and S62C. In certain embodiments, the pair of mutated cysteine residues can be a pair of substitutions selected from the group consisting of: S16C in CBeta and Y11C in CAlpha, F13C in CBeta and L13C in CAlpha, S16C in CBeta and L13C in CAlpha, V12C in CBeta and S16C in CAlpha, E14C in CBeta and S16C in CAlpha, F13C in CBeta and V23C in CAlpha, L62C in CBeta and Y44C in CAlpha, D58C in CBeta and T46C in CAlpha, S76C in CBeta and T46C in CAlpha, S56C in CBeta and T49C in CAlpha, S56C in CBeta and L51C in CAlpha, S56C in CBeta and S62C in CAlpha, and R78C in CBeta and S62C in CAlpha, and wherein the pair of cysteine residues are capable of forming a non-native interchain disulphide bond.

In certain embodiments, the engineered CAlpha and/or CBeta comprise one or more mutated residues to form one or more non-native disulfide bonds, selected from: P8C on CAlpha, A9C on CAlpha, V10C on CAlpha, F26C on CAlpha, F29C on CAlpha, T33C on CAlpha, Q34C on CAlpha, V35C on CAlpha, S36C on CAlpha, S38C on CAlpha, K39C on CAlpha, F78C on CAlpha, N80C on CAlpha, S81C on CAlpha, I82C on CAlpha, P84C on CAlpha, D86C on CAlpha, T87C on CAlpha, F88C on CAlpha, F89C on CAlpha, P90C on CAlpha, and A18C on CBeta.

In certain embodiments, the engineered CAlpha and/or CBeta comprise one or more groups of mutated residues to form non-native disulfide bonds, selected from: F26C and F78C on CAlpha, S36C and N80C on CAlpha, S38C and N80C on CAlpha, K39C and N80C on CAlpha, Q34C and S81C on CAlpha, V35C and S81C on CAlpha, S36C and S81C on CAlpha, Q34C and I82C on CAlpha, P8C and P84C on CAlpha, F29C and P84C on CAlpha, T33C and P84C on CAlpha, P8C and D86C on CAlpha, P8C and T87C on CAlpha, A9C and F88C on CAlpha, V10C and F89C on CAlpha, P90C on CAlpha and A18C on CBeta, P8C, D86C, P90C on CAlpha and A18C on CBeta, P8C and D86C on CAlpha.

As used herein throughout the application, “XnY” with respect to a TCR constant region is intended to mean that the n^{th} amino acid residue X on the TCR constant region (based on the numbering in FIGS. 12-13 as provided herein) is replaced by amino acid residue Y, where X and Y are respectively the one-letter abbreviation of a particular amino acid residue. It should be noted that the number n is solely based on the numbering provided in FIGS. 12-13, and it could appear different from its actual position.

In addition to the non-native amino acid residue, the engineered TCR constant region in certain embodiments may further comprise an additional modification to one or more native residues in the wild-type TCR constant region sequence. Examples of such additional modification include, such as modification to a native cysteine residue, modification to a native glycosylation site, and/or modification to a native loop.

In certain embodiments, at least one native cysteine residue is absent or present in the engineered CBeta. For example, the native cysteine residue at position C74 of CBeta may be present or absent in the engineered CBeta.

Without wishing to be bound by any theory, it is believed that the polypeptide complex provided herein is advantageous in that it tolerates both presence and absence of the native cysteine residue on the CBeta. Although it was suggested (see, for example, U.S. Pat. No. 7,666,604) that presence of the native cysteine residues on soluble TCR heterodimers is detrimental to the ligand binding ability of the TCR, the polypeptide complex provided herein can tolerate presence of this native cysteine residue without negatively affecting its antigen-binding activity. Furthermore, the polypeptide complex provided herein in the absence of the native cysteine residue expressed at high level, despite of the contrary teachings by Wu et al. mAbs, 7(2), pp. 364-376 (2005) that native disulphide bond in the TCR heterodimer is good for stabilizing the TCR heterodimer.

In certain embodiments, one or more native glycosylation site present in the native TCR constant regions may be modified (e.g. removed) or kept in the polypeptide complex provided in the present disclosure. The term “glycosylation site” as used herein with respect to a polypeptide sequence refers to an amino acid residue with a side chain to which a carbohydrate moiety (e.g. an oligosaccharide structure) can be attached. Glycosylation of polypeptides like antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-acetylgalactosamine, galactose, or xylose to a hydroxyamino acid, most commonly to serine or threonine. Removal of native glycosylation sites can be conveniently accomplished by altering the amino acid sequence such that one or more of the above-described tripeptide sequences (for N-linked glycosylation sites) or one or more serine or threonine residues (for 0-linked glycosylation sites) are substituted.

In certain embodiments, in the polypeptide complex provided herein, at least one native glycosylation site is absent or present in the engineered TCR constant regions, for example, in the first and/or the second TCR constant regions. Without wishing to be bound by any theory, it is believed that the polypeptide complex provided herein can tolerate removal of all or part of the glycosylation sites without affecting the protein expression and stability, in contrast to existing teachings that presence of N-linked glycosylation sites on TCR constant region, such as CAlpha (i.e. N34, N68, and N79) and CBeta (i.e. N69) are necessary for protein expression and stability (see Wu et al., Mabs, 7:2, 364-376, 2015).

In certain embodiments, in the polypeptide complex provided herein, at least one of the N-glycosylation sites in the engineered CAlpha, e.g. N34, N68, N79 and N61 are absent or present. In certain embodiments, at least one of the N-glycosylation sites in the engineered CBeta, e.g. N69, is absent or present.

In certain embodiments, one or more native secondary structure present in the native TCR constant regions may be modified (e.g. removed) or kept in the polypeptide complex provided in the present disclosure. In certain embodiments, a native loop (such as FG loop and/or DE loop of native CBeta) is modified (e.g. removed) or kept in the polypeptide complex provided herein. The term “FG loop” and “DE loop” are structures mainly found in the TCR beta chain constant domain. The FG loop encompasses amino acid residues 101-117 of the native CBeta and is an unusually elongated, solvent-exposed structural element that forms one component of an alpha/beta TCR cavity against CD3. The DE loop encompasses amino acid residues 66-71 of the native CBeta. In certain embodiments, the sequence at FG loop is absent and/or replaced with YPSN (SEQ ID NO: 81). In certain embodiments, the sequence at native DE loop is absent and/or replaced with QSGR (SEQ ID NO: 82).

In certain embodiments, the engineered CAlpha and CBeta comprise at least one residue substituted with at least one corresponding amino acid residue that contributes to the stability of murine TCR, to improve the stability and/or expression level of the polypeptide complex disclosed herein.

In certain embodiments, the engineered CAlpha comprises one or more mutated residues, e.g., 1, 2, 3, 4, 5, or 6 mutated residues, at the C-terminus, wherein mutated residues comprise a fragment derived from a human IgG1 hinge sequence, a human kappa light c-terminal sequence, a PDB-5DK3 (human IgG4) CH1 region sequence, a human lambda light chain c-terminus region sequence, a PDB-1IGT (mouse IgG2a) CH1 region sequence, a PDB-1IGT (mouse IgG2a) kappa light chain c-terminus region sequence, a human IgG2 hinge region sequence (e.g., ERKCC-ERKSC, SEQ ID NO: 3), PDB-5E8E (human IgA) CH1 region sequence, a PDB-5E8E (human IgA) kappa light chain c-terminus region sequence, a PDB-1DEE (human IgM) CH1 region sequence, a human IgE CH1 region sequence, or a human IgD CH1 region sequence.

In certain embodiments, one or more modifications of CAlpha, for example at its C-terminus, may improve the stability and/or expression level of the polypeptide complex disclosed herein. In certain embodiments, replacing the CAlpha c-terminus residues (residues at the c-terminal region of CAlpha, e.g., amino acid residues at positions 81-96, 84-95, 92-95, or 92-96) by a human IgG1 sequence segment or human kappa light c-terminal segment may improve the stability and/or expression level of the polypeptide complex disclosed herein. In certain embodiments, the engineered CAlpha comprises mutations at the C-terminus of CAlpha wherein the C-terminus comprises an amino acid sequence, e.g., “EPKS” (SEQ ID NO: 4) and “VEPKS” (SEQ ID NO: 5) derived from a human IgG1 hinge sequence. For example, the engineered CAlpha comprises mutations at the C-terminus of CAlpha with “VEPKS” (SEQ ID NO: 5) in place of “PESS” (SEQ ID NO: 6). For another example, the engineered CAlpha comprises mutations at the C-terminus of CAlpha with “EPKS” (SEQ ID NO: 4) in place of “PESS” (SEQ ID NO: 6). In certain embodiments, the engineered CAlpha comprises mutations at the C-terminus of CAlpha wherein the C-terminus comprises an amino acid sequence, e.g., “NRGE” (SEQ ID NO: 7), derived from human kappa light c-terminal residues. For example, the engineered CAlpha comprises mutations at the C-terminus of CAlpha with “NRGE” (SEQ ID NO: 7) in place of “PESS” (SEQ ID NO: 6).

In certain embodiments, modifications of at least one amino acid residue on CBeta, for example, the residues that are structurally or spatially close to the C-terminus of CAlpha, may also improve the stability and/or expression level of the polypeptide complexes disclosed herein.

In certain embodiments, the engineered CAlpha and/or CBeta comprise one or more residues mutated to corresponding one or more amino acid residues from murine TCR to improve stability and/or expression levels of the polypeptide complexes disclosed herein. For example, the engineered CAlpha comprises at least one mutated residue selected from substitutions P92S, E93D, S94V, and S95P, and/or the engineered CBeta comprises at least one mutated residue selected from substitutions E17K and S21A. In certain embodiments, the engineered CAlpha comprises mutated residues P92S, E93D, S94V, and S95P. In certain embodiments, the engineered CBeta comprises mutated residues E17K and S21A. In certain embodiments, the engineered CAlpha comprises mutated residues P92S, E93D, S94V, and S95P, and the engineered CBeta comprises mutated residues E17K and S21A.

In certain embodiments, the engineered CAlpha and/or the engineered CBeta comprise one, two, three, four, five, six, or seven mutations chosen from S22F, T33L, and A73T on CAlpha and E17K, H22R, D38P, and S53D on TCR CBeta.

In the polypeptide complex provided herein, the constant regions derived from a TCR are operably linked to the variable regions derived from an antibody. The heavy chain or light chain variable region of an antibody can be operably linked to a TCR constant region, with or without a spacer.

In certain embodiments, the first antibody heavy chain variable domain (VH) is fused to the first TCR constant region (C1) at a first conjunction domain, the first antibody light chain variable domain (VL) is fused to the second TCR constant region (C2) at a second conjunction domain.

In certain embodiments, the first conjunction domain comprises at least a portion of the C terminal fragment of an antibody V/C conjunction, and at least a portion of the N-terminal fragment of a TCR V/C conjunction.

The term “antibody V/C conjunction” as used herein refers to the boundary of antibody variable domain and constant domain, for example, the boundary between heavy chain variable domain and the CH1 domain, or between light chain variable domain and the light chain constant domain. Similarly, the term “TCR V/C conjunction” refers to the boundary of TCR variable domain and constant domain, for example, the boundary between TCR Alpha variable domain and constant domain, or between TCR Beta variable domain and constant domain.

Conjunction domains, such as the first conjunction domain and the second conjunction domain, are described in PCT/CN2018/106766 (WO 2019/057122) and are incorporated herein by reference.

In certain embodiments, the first polypeptide comprises a sequence comprising domains operably linked as in formula (I): VH-HCJ-C1, and the second polypeptide comprises a sequence comprising domains operably linked as in formula (II): VL-LCJ-C2, wherein:
VH is a heavy chain variable domain of an antibody;
HCJ is a first conjunction domain as defined supra;
C1 is a first TCR constant domain as defined supra;
VL is a light chain variable domain of an antibody;
LCJ is a second conjunction domain as defined supra;
C2 is a second TCR constant domain as defined supra.

Various sequences of HCJ and LCJ are described in PCT/CN2018/106766 (WO 2019/057122) and are incorporated herein by reference.

U.S. Pat. No. 9,683,052 discloses that certain residues within the contact interface between TCR constant regions can be engineered into an Fc region to facilitate the hetero-dimeric pairing of two heavy chains. Such residues and/or corresponding residues within the contact interface between TCR constant regions disclosed herein can also be engineered into a Fab region, e.g., the CH1 and CL domains, to facilitate the pairing between a light chain and a heavy chain.

ii. Antibody Variable Region

The polypeptide complex provided herein comprises a first heavy chain variable domain (VH) and a first light chain variable domain (VL) of the first antibody. In a conventional native antibody, a variable region comprises three CDR regions interposed by flanking framework (FR) regions, for example, as set forth in the following formula: FR1-CDR1-FR2-CDR2-FR3-CDR3-FR4, from N-terminus to C-terminus. The polypeptide complex provided herein can comprise but is not limited to such a conventional antibody variable region. For example, the variable domain may comprise all three or less than three of the CDRs, with all four or less than four of the FRs of the antibody heavy or light chain, as long as the variable domain is capable of specifically binding to an antigen.

The first antibody has a first antigenic specificity. In other words, VH and VL form an antigen-binding site which can specifically bind to an antigen or an epitope. The antigenic specificity can be directed to any suitable antigen or epitope, for example, one that is exogenous antigen, endogenous antigen, autoantigen, neoantigen, viral antigen or tumor antigen. An exogenous antigen enters a body by inhalation, ingestion or injection, and can be presented by the antigen-presenting cells (APCs) by endocytosis or phagocytosis and form MHC II complex. An endogenous antigen is generated within normal cells as a result of cell metabolism, intracellular viral or bacterial infection, which can form MHC I complex. An autoantigen (e.g. peptide, DNA or RNA, etc.) is recognized by the immune system of a patient suffering from autoimmune diseases, whereas under normal condition, this antigen should not be the target of the immune system. A neoantigen is entirely absent from the normal body, and is generated because of a certain disease, such as tumor or cancer. In certain embodiments, the antigen is associated with a certain disease (e.g. tumor or cancer, autoimmune diseases, infectious and parasitic diseases, cardiovascular diseases, neuropathies, neuropsychiatric conditions, injuries, inflammations, coagulation disorder). In certain embodiments, the antigen is associated with immune system (e.g. immunological cells such as B cell, T cell, NK cells, macrophages, etc.).

In certain embodiments, the first antigenic specificity is directed to an immune-related antigen or an epitope thereof. Examples of an immune-related antigen include a T-cell specific receptor molecule and/or a natural killer cell (NK cell) specific receptor molecule.

The T-cell specific receptor molecule allows a T cell to bind to and, if additional signals are present, to be activated by and respond to an epitope/antigen presented by another cell called the antigen-presenting cell or APC. The T-cell specific receptor molecule can be TCR, CD3, CD28, CD134 (also termed OX40), 4-1 BB, CD5, and CD95 (also known as the Fas receptor).

Examples of a NK cell specific receptor molecule include CD16, a low affinity Fc receptor and NKG2D, and CD2.

In certain embodiments, the first antigenic specificity is directed to a tumor-associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal, i.e. non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but is overexpressed on tumor cells when compared to non-tumor cells or are accessible for antibody binding in tumor cells due to the less compact structure of the tumor tissue compared to non-tumor tissue. In some embodiments the tumor associated antigen is located on the vasculature of a tumor. Illustrative examples of a tumor associated surface antigen are described in PCT/CN2018/106766 (WO 2019/057122) and are incorporated herein by reference.

In certain embodiments, the first antigenic specificity is directed to an antigen or an epitope thereof, selected from the group consisting of: CD3, 4.1BB (CD137), OX40 (CD134), CD16, CD47, CD19, CD20, CD22, CD33, CD38, CD123, CD133, CEA, cdH3, EpCAM, epidermal growth factor receptor (EGFR), EGFRvIII (a mutant form of EGFR), HER2, HER3, dLL3, BCMA, Sialyl-Lea, 5T4, ROR1, melanoma-associated chondroitin sulfate proteoglycan, mesothelin, folate receptor 1, VEGF receptor, EpCAM, HER2/neu, HER3/neu, G250, CEA, MAGE, proteoglycans, VEGF, FGFR, alphaVbeta3-integrin, HLA, HLA-DR, ASC, CD1, CD2, CD4, CD5, CD6, CD7, CD8, CD11, CD13, CD14, CD21, CD23, CD24, CD28, CD30, CD37, CD40, CD41, CD44, CD52, CD64, c-erb-2, CALLA, MHCII, CD44v3, CD44v6, p97, ganglioside GM1, GM2, GM3, GDla, GDlb, GD2, GD3, GT1b, GT3, GQ1, NY-ESO-1, NFX2, SSX2, SSX4 Trp2, gp100, tyrosinase, Muc-1, telomerase, survivin, G250, p53, CA125 MUC, Wue antigen, Lewis Y antigen, HSP-27, HSP-70, HSP-72, HSP-90, Pgp, MCSP, EpHA2, cell surface targets GC182, GT468 or GT512, IL-17, IL-20, IL-13, and IL-4.

The antibody variable domains can be derived from a parent antibody. A parent antibody can be any type of antibody, including for example, a fully human antibody, a humanized antibody, or an animal antibody (e.g. mouse, rat, rabbit, sheep, cow, dog, etc.). The parent antibody can be a monoclonal antibody or a polyclonal antibody.

In certain embodiments, the parent antibody is a monoclonal antibody. A monoclonal antibody can be produced by various methods known in the art, for example, hybridoma technology, recombinant method, phage display, or any combination thereof. Exemplary methods of producing antibodies are described in PCT/CN2018/106766 (WO 2019/057122) and are incorporated herein by reference.

The parent antibodies described herein can be further modified, for example, to graft the CDR sequences to a different framework or scaffold, to substitute one or more amino acid residues in one or more framework regions, to replace one or more residues in one or more CDR regions for affinity maturation, and so on. These can be accomplished by a person skilled in the art using conventional techniques.

The parent antibody can also be a therapeutic antibody known in the art, for example those approved by FDA for therapeutic or diagnostic use, or those under clinical trial for treating a condition, or those in research and development. Polynucleotide sequences and protein sequences for the variable regions of known antibodies can be obtained from public databases such as, for example, e.g., www.ncbi.nlm nih gov/entrez-/query.fcgi; www.atcc.org/phage/hdb.html; www.sciquest.com/; www.abcam.com/; www.antibodyresource.com/onlinecomp.html.Examples of therapeutic antibodies include, but are not limited to the therapeutic antibodies disclosed in PCT/CN2018/106766 (WO 2019/057122) and are incorporated herein by reference.

In certain embodiments, the first antigen-binding moiety binds to CTLA-4. In certain embodiments, the second antigen-binding moiety binds to PD-L1. In certain embodiments, the first antigen-binding moiety binds to PD-L1. In certain embodiments, the second antigen-binding moiety binds to CTLA-4. In certain embodiments, the first antigen-binding moiety binds to PD-L1. In certain embodiments, the second antigen-binding moiety binds to 4-1BB. In certain embodiments, the first antigen-binding moiety binds to 4-1BB. In certain embodiments, the second antigen-binding moiety binds to PD-L1. In certain embodiments, the first and second antigen-binding moieties binds to two separate domains of HER2. In certain embodiments, the first antigen-binding moiety binds to HER2 D2. In certain embodiments, the second antigen-binding moiety binds to HER2 D4. In certain embodiments, the first antigen-binding moiety binds to HER2 D4. In certain embodiments, the second antigen-binding moiety binds to HER2 D2. In certain embodiments, the first antigen-binding moiety binds to IL-17. In certain embodiments, the second antigen-binding moiety binds to IL-20. In certain embodiments, the first antigen-binding moiety binds to IL-20. In certain embodiments, the second antigen-binding moiety binds to IL-17. In certain embodiments, the first antigen-binding moiety binds to IL-4. In certain embodiments, the second antigen-binding moiety binds to IL-13. In certain embodiments, the first antigen-binding moiety binds to IL-13. In certain embodiments, the second antigen-binding moiety binds to IL-4.

CDRs are known to be responsible for antigen binding, however, it has been found that not all of the 6 CDRs are indispensable or unchangeable. In other words, it is possible to replace or change or modify one or more CDRs, yet substantially retain the specific binding affinity to the antigen.

### Bispecific Polypeptide Complexes

In one aspect, the present disclosure provides herein a bispecific polypeptide complex. The term “bispecific” as used herein means that, there are two antigen-binding moieties, each of which is capable of specifically binding to a different antigen or a different epitope on the same antigen. The bispecific polypeptide complex provided herein comprises a first antigen-binding moiety comprising a first heavy chain variable domain operably linked to a first TCR constant region (C1) and a first light chain variable domain operably linked to a second TCR constant region (C2), wherein C1 and C2 are capable of forming a dimer comprising at least one non-native and stabilizing interchain bond between C1 and C2. The bispecific polypeptide complex provided herein further comprises a second antigen-binding moiety comprising a second antigen-binding site but does not contain a sequence derived from a TCR constant region.

In certain embodiments, the present disclosure provides a bispecific polypeptide complex, comprising a first antigen-binding moiety associated with a second antigen-binding moiety, wherein:
the first antigen-binding moiety comprising:
   a first polypeptide comprising, from N-terminus to C-terminus, a first heavy chain variable domain (VH) of a first antibody operably linked to a first T cell receptor (TCR) constant region (C1), and
   a second polypeptide comprising, from N-terminus to C-terminus, a first light chain variable domain (VL) of the first antibody operably linked to a second TCR constant region (C2),
wherein:
   C1 comprises an engineered CBeta, and C2 comprises an engineered CAlpha; or C1 comprises an engineered CAlpha, and C2 comprises an engineered CBeta,
   C1 and C2 are capable of forming a dimer comprising at least one non-native interchain bond between a first mutated residue comprised in C1 and a second mutated residue comprised in C2, and the non-native interchain bond is capable of stabilizing the dimer,
   the CAlpha and/or CBeta comprises at least one mutation at the C-terminal regions of CAlpha and/or CBeta or at amino acid positions that are spatially close to the c-terminus of CAlpha and/or CBeta to further stabilize the dimer, and
   the first antibody has a first antigenic specificity,
   a second antigen-binding moiety has a second antigenic specificity which is different from the first antigenic specificity.

The bispecific polypeptide complex provided herein is significantly less prone to have mispaired heavy chain and light chain variable domains, have increased stability, and/or expression level.

In certain embodiments, the second antigen-binding moiety of the bispecific polypeptide complex provided herein comprises a second heavy chain variable domain (VH2) and a second light chain variable domain (VL2) of a second antibody. In certain embodiments, at least one of VH2 and VL2 is operably linked to an antibody constant region, or both VH2 and VL2 are operably linked to antibody heavy chain and light chain constant regions respectively. In certain embodiments, the second antigen-binding moiety further comprises an antibody constant CH1 domain operably linked to VH2, and an antibody light chain constant domain operably linked to VL2. For example, the second antigen-binding moiety comprises a Fab.

Where a first, second, third, and fourth variable domains (e.g. VH1, VH2, VL1 and VL2) are expressed in one cell, it is highly desired that VH1 specifically pairs with VL1, and VH2 specifically pairs with VL2, such that the resulting bispecific protein product would have the correct antigen-binding specificities. However, in existing technologies such as hybrid-hybridoma (or quadroma), random pairing of VH1, VH2, VL1 and VL2 occurs and consequently results in generation of up to ten different species, of which only one is the functional bispecific antigen-binding molecule. This not only reduces production yields but also complicates the purification of the target product.

In an illustrative example, the first antigen-binding domain comprises VH1-C1 paired with VL1-C2, and the second antigen-binding domain comprises VH2-CH1 paired with VL2-CL. It has been surprisingly found that C1 and C2 preferentially associates with each other, and are less prone to associate with CL or CH1, thereby formation of unwanted pairs such as C1-CH, C1-CL, C2-CH, and C2-CL are discouraged and significantly reduced. As a result of specific association of C1-C2, VH1 specifically pairs with VL1 and thereby rendering the first antigen binding site, and CH1 specifically pairs with CL, thereby allowing specific pairing of VH2-VL2 which provides for the second antigen binding site. Accordingly, the first antigen binding moiety and the second antigen binding moiety are less prone to mismatch, and mispairings between for example VH1-VL2, VH2-VL1, VH1-VH2, VL1-VL2 would be significantly reduced than otherwise could have been if both the first and the second antigen-binding moieties are counterparts of natural Fabs, e.g. in the form of VH1-CH1, VL1-CL, VH2-CH1, and VL2-CL.

In certain embodiments, the bispecific polypeptide complex provided herein, when expressed from a cell, would have significantly less mispairing products (e.g., at least 1, 2, 3, 4, 5 or more mispairing products less) and/or significantly higher production yield (e.g., at least 10%, 20%, 30%, 40%, 50%, 60% or more higher yield), than a reference molecule expressed under comparable conditions, wherein the reference molecule is otherwise identical to the bispecific polypeptide complex except having a native CH1 in the place of C1 and a native CL in the place of C2.

In certain embodiments, the first and/or the second antigen binding moiety is multivalent, such as bivalent, trivalent, tetravalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope). In certain embodiments, the first and/or the second antigen-binding moiety in the bispecific polypeptide complex provided herein can be bivalent, trivalent, or tetravalent, with at least two binding sites specific for the same antigen or epitope. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences), or structurally different (i.e. having different sequences albeit with the same specificity).

In certain embodiments, the first and/or the second antigen binding moiety is multivalent and comprises two or more antigen binding sites operably linked together, with or without a spacer.

In certain embodiments, the first and/or the second antigen binding moiety comprises one or more of Fab, Fab′, Fab′-SH, F(ab′)₂, Fd, Fv, and scFv fragments, and other fragments described in Spiess et al., 2015, supra and Brinkmann et al., 2017, supra, or the combination thereof, which are linked with or without a spacer at the heavy chain and/or the light chain and forms at least one antigen binding moiety.

In certain embodiments, the second antigen binding moiety comprises two or more Fab of the second antibody. The two Fabs can be operably linked to each other, for example the first Fab can be covalently attached to the second Fab via heavy chain, with or without a spacer in between.

In certain embodiments, the first antigen-binding moiety further comprises a first dimerization domain, and the second antigen-binding moiety further comprises a second dimerization domain. The term “dimerization domain” as used herein refers to the peptide domain which is capable of associating with each other to form a dimer, or in some examples, enables spontaneous dimerization of two peptides.

In certain embodiments, the first dimerization domain can be associated with the second dimerization domain. The association can be via any suitable interaction or linkage or bonding, for example, via a connecter, a disulphide bond, a hydrogen bond, electrostatic interaction, a salt bridge, or hydrophobic-hydrophilic interaction, or the combination thereof. Exemplary dimerization domains include, without limitation, antibody hinge region, an antibody CH2 domain, an antibody CH3 domain, and other suitable protein monomers capable of dimerizing and associating with each other. Hinge region, CH2 and/or CH3 domain can be derived from any antibody isotypes, such as IgG1, IgG2, and IgG4.

In certain embodiments, the first and/or the second dimerization domain comprises at least a portion of an antibody hinge region. In certain embodiments, the first and/or the second dimerization domain may further comprise an antibody CH2 domain, and/or an antibody CH3 domain. In certain embodiments, the first and/or the second dimerization domain comprises at least a portion of Hinge-Fc region, i.e. Hinge-CH2-CH3 domain. In certain embodiments, the first dimerization domain can be operably linked to the C terminal of the first TCR constant region. In certain embodiments, the second dimerization domain can be operably linked to the C terminal of the antibody CH1 constant region of the second antigen-binding moiety.

In certain embodiments, the first dimerization domain is operably linked to (with or without a spacer in between) the first TCR constant region (C1) at a third conjunction domain. Various designs and sequences of the third conjunction domain are described in PCT/CN2018/106766 (WO 2019/057122) and are incorporated herein by reference.

In certain embodiments, the first dimerization domain is operably linked to the C-terminal of an engineered TCR constant region, and together forms a chimeric constant region. In other words, the chimeric constant region comprises the first dimerization domain operably linked with the engineered TCR constant region.

In certain embodiments, the chimeric constant region comprises an engineered CBeta attached to the first hinge-Fc region derived from IgG1, IgG2 or IgG4. Exemplary sequences of such a chimeric constant region are described in in PCT/CN2018/106766 (WO 2019/057122) and are incorporated herein by reference.

In certain embodiments, the chimeric constant region comprises an engineered CAlpha attached to the first hinge derived from IgG1, IgG2 or IgG4. Exemplary sequences of such chimeric constant region are described in in PCT/CN2018/106766 (WO 2019/057122) and are incorporated herein by reference.

In certain embodiments, the chimeric constant region further comprises a first antibody CH2 domain, and/or a first antibody CH3 domain. For example, the chimeric constant region further comprises a first antibody CH2-CH3 domain attached to the C-terminus of the third conjunction domain. Exemplary sequences of such chimeric constant region are described in in PCT/CN2018/106766 (WO 2019/057122) and are incorporated herein by reference.

These pairs of chimeric constant regions and second TCR constant domains are useful in that they can be manipulated to fuse to a desired antibody variable region, so as to provide for the polypeptide complex as disclosed herein. For example, an antibody heavy chain variable region can be fused to the chimeric constant region (comprising C1), thereby rendering the first polypeptide chain of the polypeptide complex provided herein; and similarly, an antibody light chain variable region can be fused to the second TCR constant domain (comprising C2), thereby rendering the second polypeptide chain of the polypeptide complex provided herein.

These pairs of chimeric constant regions and second TCR constant domains can be used as a platform for generating the first antigen-binding moiety of the bispecific polypeptide complexes provided herein. For example, variable regions of a first antibody can be fused at the N-terminus of the platform sequences (e.g. fusing the VH to the chimeric constant domain and the VL to the TCR constant domain, respectively). To produce the bispecific polypeptide complex, the second antigen-binding moiety can be designed and produced, so as to associate into the bispecific polypeptide complex provided herein.

In certain embodiments, the second dimerization domain comprises a hinge region. The hinge region may be derived from an antibody, such as IgG1, IgG2, or IgG4. In certain embodiments, the second dimerization domain may optionally further comprise an antibody CH2 domain, and/or an antibody CH3 domain, for example such as a hinge-Fc region. The hinge region may be attached to the antibody heavy chain of the second antigen binding site (e.g., Fab).

In the bispecific polypeptide complex, the first and the second dimerization domain are capable of associating into a dimer. In certain embodiments, the first and the second dimerization domains are different and associate in a way that discourages homodimerization and/or favors heterodimerization. For example, the first and the second dimerization domains can be selected so that they are not identical and that they preferentially form heterodimers between each other rather than to form homodimers within themselves. In certain embodiments, the first and the second dimerization domains are capable of associating into heterodimers via formation of knob-into-hole, hydrophobic interaction, electrostatic interaction, hydrophilic interaction, or increased flexibility.

In certain embodiments, the first and the second dimerization domains comprise CH2 and/or CH3 domains which are respectively mutated to be capable of forming a knobs-into-holes. A knob can be obtained by replacement of a small amino acid residue with a larger one in the first CH2/CH3 polypeptide, and a hole can be obtained by replacement of a large residue with a smaller one. For details of the mutation sites for knobs into holes please see Ridgway et al., 1996, supra, Spiess et al., 2015, supra and Brinkmann et al., 2017, supra.

In certain embodiments, the first and the second dimerization domains comprise a first CH3 domain of the IgG1 isotype containing S139C and T151W substitution (knob) and a second CH3 domain of the IgG1 isotype containing Y134C, T151S, L153A and Y192V substitution (hole). In another embodiments, the first and the second dimerization domains comprise a first CH3 domain of the IgG4 isotype containing S136C and T148W substitution (knob) and a second CH3 domain of the IgG4 isotype containing Y131C, T148S, L150A and Y189V substitution (hole). Further illustrative examples of the first and the second dimerization domains are described in PCT/CN2018/106766 (WO 2019/057122) and are incorporated herein by reference.

In certain embodiments, the first and the second dimerization domains further comprise a first hinge region and a second hinge region. For example, charge pairs of substitution can be introduced into the hinge region of IgG1 and IgG2 to promote heterodimerization. For details see Brinkmann et al., 2017, supra.

Bispecific format

The bispecific polypeptide complex provided herein can be in any suitable bispecific format known in the art. In certain embodiments, the bispecific polypeptide complex is based on a reference bispecific antibody format. “Based on” as used herein with respect to a bispecific format means that the bispecific polypeptide complex provided herein takes the same bispecific format of a reference bispecific antibody, except that one of the antigen-binding moiety has been modified to comprise a VH operably linked to C1 and a VL operably linked to C2 wherein C1 and C2 are associated as disclosed herein. Examples of reference bispecific antibody formats known in the art include, without limitation, (i) a bispecific antibody with symmetric Fc, (ii) a bispecific antibody with asymmetric Fc, (iii) a regular antibody appended with an additional antigen-binding moiety, (iv) a bispecific antibody fragment, (v) a regular antibody fragment appended with an additional antigen-binding moiety, (vi) a bispecific antibody appended with human albumin or human albumin-binding peptide.

BsIgG is monovalent for each antigen and can be produced by co-expression of the two light and two heavy chains in a single host cell. An appending IgG is engineered to form bispecific IgG by appending either the amino or carboxy termini of either light or heavy chains with additional antigen-binding units. The additional antigen-binding units can be single domain antibodies (unpaired VL or VH), such as DVD-Ig, paired antibody variable domains (e.g. Fv or scFv) or engineered protein scaffolds. Any of the antigen-binding units in BsIgG, in particular paired VH-CH1/VL-CL, can be modified to replace the CH1 to C1 and CL to C2 as disclosed herein, to render the bispecific polypeptide complex as provided herein.

Bispecific antibody fragments are antigen-binding fragments that are derived from an antibody but lack some or all of the antibody constant domains. Examples of such a bispecific antibody fragment include, for example, such as single domain antibody, Fv, Fab and diabody etc. To render the bispecific polypeptide complex as provided herein, an antigen-binding site (e.g. particular paired VH-CH1/VL-CL) in a bispecific antibody fragment, can be modified to comprise the polypeptide complex as disclosed herein (e.g. VH-C1/CL-C2).

In certain embodiments, the bispecific polypeptide complex as provided herein is based on the format of a “whole” antibody, such as whole IgG or IgG-like molecules, and small recombinant formats, such as tandem single chain variable fragment molecules (taFvs), diabodies (Dbs), single chain diabodies (scDbs) and various other derivatives of these (cf. bispecific antibody formats as described by Byrne H. et al. (2013) Trends Biotech, 31 (11): 621-632. Examples of bispecific antibody is based on a format which include, but is not limited to, quadroma, chemically coupled Fab (fragment antigen binding), and BiTE (bispecific T cell engager).

In certain embodiments, the bispecific polypeptide complex as provided herein is based on a bispecific format selected from Triomabs; hybrid hybridoma (quadroma); Multispecific anticalin platform (Pieris); Diabodies; Single chain diabodies; Tandem single chain Fv fragments; TandAbs, Trispecific Abs (Affimed); Darts (dual affinity retargeting; Macrogenics); Bispecific Xmabs (Xencor); Bispecific T cell engagers (Bites; Amgen; 55 kDa); Triplebodies; Tribody (Fab-scFv) Fusion Protein (CreativeBiolabs) multifunctional recombinant antibody derivates; Duobody platform (Genmab); Dock and lock platform; Knob into hole (KIH) platform; Humanized bispecific IgG antibody (REGN1979) (Regeneron); Mab2 bispecific antibodies (F-Star); DVD-Ig (dual variable domain immunoglobulin) (Abbvie); kappa-lambda bodies; TBTI (tetravalent bispecific tandem Ig); and CrossMab.

In certain embodiments, the bispecific polypeptide complex as provided herein is based on a bispecific format selected from bispecific IgG-like antibodies (BsIgG) comprising CrossMab; DAF (two-in-one); DAF (four-in-one); DutaMab; DT-IgG; Knobs-in-holes common LC; Knobs-in-holes assembly; Charge pair; Fab-arm exchange; SEEDbody; Triomab; LUZ-Y; Fcab; kappa-lamda-body; and Orthogonal Fab. For detailed description of the bispecific antibody formats please see Spiess C., Zhai Q. and Carter P. J. (2015) Molecular Immunology 67: 95-106, which is incorporated herein by reference in its entirety.

In certain embodiments, the bispecific polypeptide complex as provided herein is based on a bispecific format selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; KIH IgG-scFab; 2scFv-IgG; IgG-2scFv; scFv4-Ig; scFv4-Ig; Zybody; and DVI-IgG (four-in-one) (see Id.).

In certain embodiments, the bispecific polypeptide complex as provided herein is based on a format selected from bispecific antibody fragments comprising Nanobody; Nanobody-HAS; BiTE; Diabody; DART; TandAb; scDiabody; sc-Diabody-CH3; Diabody-CH3; Triple Body; Miniantibody; Minibody; TriBi minibody; scFv-CH3 KIH; Fab-scFv; scFv-CH-CL-scFv; F(ab′)2; F(ab′)2-scFv2; scFv-KIH; Fab-scFv-Fc; Tetravalent HCAb; scDiabody-Fc; Diabody-Fc; Tandem scFv-Fc; and Intrabody (see Id).

In certain embodiments, the bispecific polypeptide complex as provided herein is based on a bispecific format such as Dock and Lock; ImmTAC; HSAbody; scDiabody-HAS; and Tandem scFv-Toxin (see Id.).

In certain embodiments, the bispecific polypeptide complex as provided herein is based on a format selected from bispecific antibody conjugates comprising IgG-IgG; Cov-X-Body; and scFv1-PEG-scFv2 (see Id.).

In certain embodiments, the first antigen-binding moiety and the second binding moiety can be associated into an Ig-like structure. An Ig-like structure is like a natural antibody having a Y shaped construct, with two arms for antigen-binding and one stem for association and stabilization. The resemblance to natural antibody can provide for various advantages such as good in vivo pharmacokinetics, desired immunological response and stability etc. It has been found that the Ig-like structure comprising the first antigen-binding moiety provided herein associated with the second antigen-binding moiety provided herein has thermal stability which is comparable to that of an Ig (e.g. an IgG). In certain embodiments, the Ig-like structure provided herein is at least 70%, 80%, 90%, 95% or 100% of that of a natural IgG.

In certain embodiments, the bispecific polypeptide complex comprises four polypeptide chains: i) VH1-C1-Hinge-CH2-CH3; ii) VL1-C2; iii) VH2-CH1-Hinge-CH2-CH3, and iv) VL2-CL, wherein the C1 and C2 are capable of forming a dimer comprising at least one non-native interchain bond, and the two hinge regions and/or the two CH3 domains are capable of forming one or more interchain bond that can facilitate dimerization. See, e.g., FIG. 14 (E17R). In certain embodiments, the bispecific polypeptide complex comprises a pair of three polypeptide chains: i) VH1-CH1-VH2-C1-Hinge-CH2-CH3; ii) VL2-C2; and iii) VL1-CL, wherein the C1 and C2 are capable of forming a dimer comprising at least one non-native interchain bond, and the two hinge regions and/or the two CH3 domains are capable of forming one or more interchain bond that can facilitate dimerization. See, e.g., FIG. 14 (G25R).

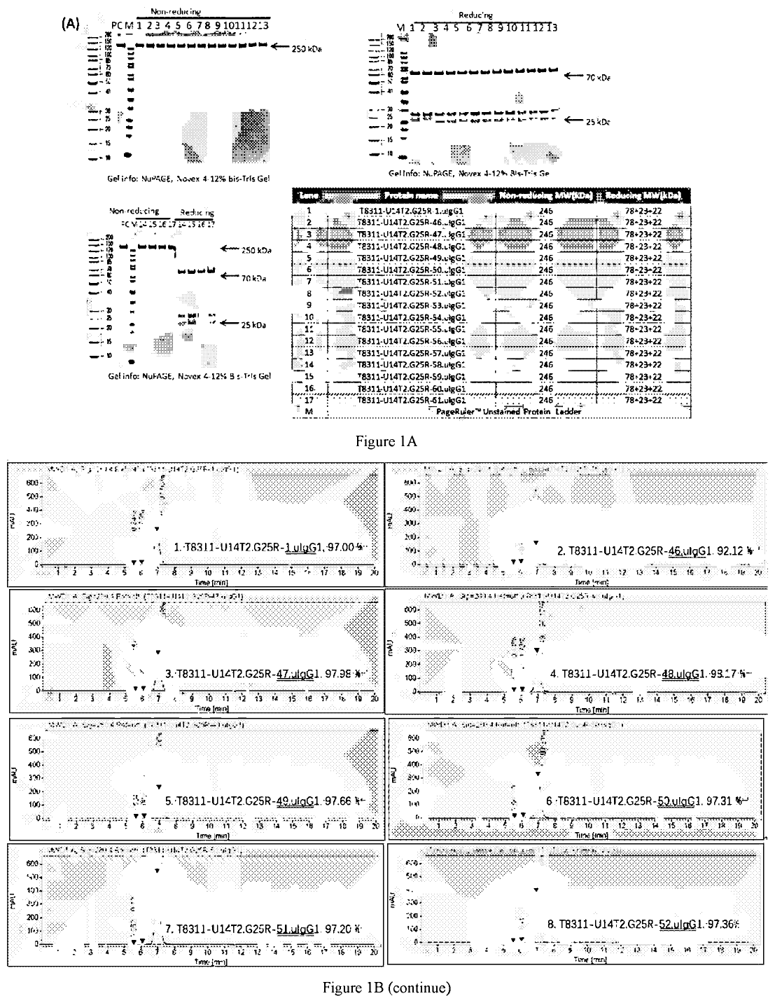

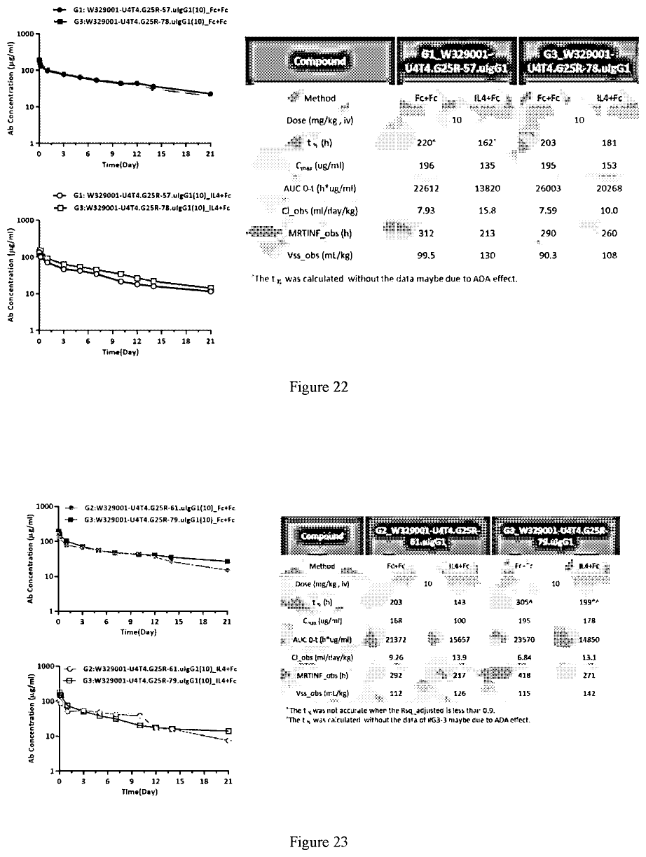

PCT/CN2018/106766 (WO 2019/057122, e.g., FIGS. 1 and 22 therein) illustrates a number of bispecific antibody formats, which are incorporated herein by reference. The engineered CAlpha and CBeta disclosed herein can be applied to any of the bispecific antibody formats as described in PCT/CN2018/106766 (WO 2019/057122).

### Antigenic Specificities of the Bispecific Complex

The bispecific complex provided herein have two antigenic specificities. The first and the second antigen-binding moieties are directed to the first and the second antigenic specificities respectively.

The first and the second antigenic specificities may be identical, in other words, the first and the second antigen-binding moieties binds to the same antigen molecule, or to the same epitope of the same antigen molecule.

Alternatively, the first and the second antigenic specificities may be distinct. For example, the first and the second antigen-binding moieties can bind to different antigens. Such a bispecific polypeptide complex could be useful in, for example, bringing the two different antigens into close proximity and thereby promoting their interactions (e.g. bringing immunological cells in close proximity to a tumor antigen or a pathogen antigen and hence promoting recognition or elimination of such an antigen by the immune system). For another example, the first and the second antigen-binding moieties can bind to different (and optionally non-overlapping) epitopes of one antigen. This may be helpful in enhancing the recognition of or binding to a target antigen, in particular one which is susceptible to mutation (e.g. a viral antigen).

In some embodiments, one of the antigenic specificity of the bispecific complex provided herein is directed to a T-cell specific receptor molecule and/or a natural killer cell (NK cell) specific receptor molecule. In some embodiments, one of the first and second antigen-binding moiety is capable of specifically binding to CD3, TCR, CD28, CD16, NKG2D, Ox40, 4-1BB, CD2, CD5 or CD95, and the other is capable of specifically binding to a tumor associated antigen.

In some embodiments, one of the antigenic specificity of the bispecific complex provided herein is directed to a T-cell specific receptor molecule and/or a natural killer cell (NK cell) specific receptor molecule, and the other antigenic specificity is directed to a tumor associated surface antigen. In certain embodiments, the first antigen-binding moiety of the bispecific complex is capable of specifically binding to T-cell specific receptor molecule and/or a natural killer cell (NK cell) specific receptor molecule (such as CD3), and the second antigen-binding moiety is capable of specifically binding to a tumor associated antigen (such as CD19), or vice versa.

In certain embodiments, the bispecific polypeptide complex comprises four polypeptide chains comprising: i) VH1 operably linked to a first chimeric constant region; ii) VL1 operably linked to a second chimeric constant region; iii) VH2 operably linked to conventional antibody heavy chain constant region, and iv) VL2 operably linked to conventional antibody light chain constant region. In certain embodiments, the first chimeric constant region can comprise C1-Hinge-CH2-CH3, each as defined supra. In certain embodiments, the second chimeric constant region can comprise C2, as defined supra. In certain embodiments, the conventional antibody heavy chain constant region can comprise CH1-Hinge-CH2-CH3, each as defined supra. In certain embodiments, the conventional antibody light chain constant region can comprise CL, as defined supra.

In certain embodiments, the bispecific polypeptide complex comprises a three-sequence combination selected from the group consisting of: SEQ ID NOs: 66, 67, and 68 (Table 22), wherein the first antigen binding moiety binds to PD-L1, and the second antigen binding moiety binds to 4-1BB.

In certain embodiments, the bispecific polypeptide complex comprises a four-sequence combination selected from the group consisting of: SEQ ID NOs: 69, 70, 71, and 72 (Table 23), wherein the first antigen binding moiety binds to HER2 D2, and the second antigen binding moiety binds to HER2 D4.

In certain embodiments, the bispecific polypeptide complex comprises a three-sequence combination selected from the group consisting of: SEQ ID NOs: 73, 74 and 75 (Table 24), wherein the first antigen binding moiety binds to IL-17, and the second antigen binding moiety binds to IL-20.

In certain embodiments, the bispecific polypeptide complex comprises a three-sequence combination selected from the group consisting of: SEQ ID NOs: 76, 77 and 78 (Table 25), wherein the first antigen binding moiety binds to IL-4, and the second antigen binding moiety binds to IL-13.

### Method of Preparation

The present disclosure provides isolated nucleic acids or polynucleotides that encode the polypeptide complex, and the bispecific polypeptide complex provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The nucleic acids or polynucleotides encoding the polypeptide complex and the bispecific polypeptide complex provided herein can be constructed using recombinant techniques. To this end, DNA encoding an antigen-binding moiety of a parent antibody (such as CDR or variable region) can be isolated and sequenced using conventional procedures (e.g., by using oligonucleotide probes that are capable of binding specifically to genes encoding the heavy and light chains of the antibody). Likewise, DNA encoding a TCR constant region can also be obtained. As an example, the polynucleotide sequence encoding the variable domain (VH) and the polynucleotide sequence encoding the first TCR constant region (C1) are obtained and operably linked to allow transcription and expression in a host cell to produce the first polypeptide. Similarly, polynucleotide sequence encoding VL are operably linked to polynucleotide sequence encoding C1, so as to allow expression of the second polypeptide in the host cell. If needed, encoding polynucleotide sequences for one or more spacers are also operably linked to the other encoding sequences to allow expression of the desired product.

The encoding polynucleotide sequences can be further operably linked to one or more regulatory sequences, optionally in an expression vector, such that the expression or production of the first and the second polypeptides is feasible and under proper control.

The encoding polynucleotide sequence(s) can be inserted into a vector for further cloning (amplification of the DNA) or for expression, using recombinant techniques known in the art. In another embodiment, the polypeptide complex and the bispecific polypeptide complex provided herein may be produced by homologous recombination known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The term “vector” as used herein refers to a vehicle into which a polynucleotide encoding a protein may be operably inserted so as to bring about the expression of that protein. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

In some embodiments, the vector system includes mammalian, bacterial, yeast systems, etc., and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS420, pLexA, pACT2.2 etc., and other laboratorial and commercially available vectors. Suitable vectors may include, plasmid, or viral vectors (e.g., replication defective retroviruses, adenoviruses and adeno-associated viruses).

Vectors comprising the polynucleotide sequence(s) provided herein can be introduced to a host cell for cloning or gene expression. The phrase “host cell” as used herein refers to a cell into which an exogenous polynucleotide and/or a vector has been introduced.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors encoding the polypeptide complex and the bispecific polypeptide complex. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptide complex, the bispecific polypeptide complex provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)), such as Expi293; baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors can be cultured in conventional nutrient media modified as appropriate for inducing promoters, selecting transformants, or amplifying the cloning vectors.

For production of the polypeptide complex and the bispecific polypeptide complex provided herein, the host cells transformed with the expression vector may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In one aspect, the present disclosure provides a method of expressing the polypeptide complex and the bispecific polypeptide complex provided herein, comprising culturing the host cell provided herein under the condition at which the polypeptide complex, or the bispecific polypeptide complex is expressed.

In certain embodiments, the present disclosure provides a method of producing the polypeptide complex provided herein, comprising a) introducing to a host cell: a first polynucleotide encoding a first polypeptide comprising, from N-terminus to C-terminus, a first heavy chain variable domain (VH) of a first antibody operably linked to a first TCR constant region (C1), and a second polynucleotide encoding a second polypeptide comprising, from N-terminus to C-terminus, a first light chain variable domain (VL) of the first antibody operably linked to a second TCR constant region (C2), wherein: C1 and C2 are capable of forming a dimer comprising at least one non-native interchain bond between C1 and C2, and the non-native interchain bond is capable of stabilizing the dimer of C1 and C2, and the first antibody has a first antigenic specificity; b) allowing the host cell to express the polypeptide complex.

In certain embodiments, the present disclosure provides a method of producing the bispecific polypeptide complex provided herein, comprising a) introducing to a host cell: a first polynucleotide encoding a first polypeptide comprising, from N-terminus to C-terminus, a first heavy chain variable domain (VH) of a first antibody operably linked to a first TCR constant region (C1), a second polynucleotide encoding a second polypeptide comprising, from N-terminus to C-terminus, a first light chain variable domain (VL) of the first antibody operably linked to a second TCR constant region (C2), and one or more (e.g., one or two) additional polynucleotides encoding a second antigen-binding moiety, wherein: C1 and C2 are capable of forming a dimer comprising at least one non-native interchain bond between a first mutated residue comprised in C1 and a second mutated residue comprised in C2, and the non-native interchain bond is capable of stabilizing the dimer of C1 and C2, the first antigen-binding moiety and the second antigen-binding moiety have reduced mispairing than otherwise would have been if the first antigen-binding moiety was a natural Fab counterpart, and the first antibody has a first antigenic specificity and the second antibody has a second antigenic specificity, b) allowing the host cell to express the bispecific polypeptide complex.

In certain embodiments, the method further comprises isolating the polypeptide complex.

When using recombinant techniques, the polypeptide complex, the bispecific polypeptide complex provided herein can be produced intracellularly, in the periplasmic space, or directly secreted into the medium. If the product is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the product is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The polypeptide complex and the bispecific polypeptide complex provided herein prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

Where the polypeptide complex or the bispecific polypeptide complex provided herein comprises immunoglobulin Fc domain, then protein A can be used as an affinity ligand, depending on the species and isotype of the Fc domain that is present in the polypeptide complex. Protein A can be used for purification of polypeptide complexes based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose.

Where the polypeptide complex or the bispecific polypeptide complex provided herein comprises a CH3 domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the polypeptide complex of interest and contaminants may be subjected to low pH hydrophobic interaction chromatography using an elution buffer at a pH between about 2.5-4.5, preferably performed at low salt concentrations (e.g., from about 0-0.25M salt).

In certain embodiments, the bispecific polypeptide complex provided herein can be readily purified with high yields using conventional methods. One of the advantages of the bispecific polypeptide complex is the significantly reduced mispairing between heavy chain and light chain variable domain sequences. This reduces production of unwanted byproducts and make it possible to obtain high purity product in high yields using relatively simple purification processes.

### Derivatives

In certain embodiments, the polypeptide complex or the bispecific polypeptide complex can be used as the base of conjugation with desired conjugates.

It is contemplated that a variety of conjugates may be linked to the polypeptide complex or the bispecific polypeptide complex provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the polypeptide complex or the bispecific polypeptide complex by covalent binding, affinity binding, intercalation, coordinate binding, complexation, association, blending, or addition, among other methods.

In certain embodiments, the polypeptide complex or the bispecific polypeptide complex provided herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the polypeptide complex or the bispecific polypeptide complex may be linked to a conjugate directly, or indirectly for example through another conjugate or through a linker.

For example, the polypeptide complex or the bispecific polypeptide complex having a reactive residue such as cysteine may be linked to a thiol-reactive agent in which the reactive group is, for example, a maleimide, an iodoacetamide, a pyridyl disulphide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

For another example, the polypeptide complex or the bispecific polypeptide complex may be conjugated to biotin, then indirectly conjugated to a second conjugate that is conjugated to avidin. For still another example, the polypeptide complex or the bispecific polypeptide complex may be linked to a linker which further links to the conjugate. Examples of linkers include bifunctional coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and his-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio)pentanoate (SPP) to provide for a disulphide linkage.

The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or P-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Methods for the conjugation of conjugates to proteins such as antibodies, immunoglobulins or fragments thereof are found, for example, in U.S. Pat. Nos. 5,208,020; 6,441,163; WO2005037992; WO2005081711; and WO2006/034488, which are incorporated herein by reference to their entirety.

### Pharmaceutical Composition

The present disclosure also provides a pharmaceutical composition comprising the polypeptide complex or the bispecific polypeptide complex provided herein and a pharmaceutically acceptable carrier.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

A “pharmaceutically acceptable carrier” refers to an ingredient in a pharmaceutical formulation, other than an active ingredient, which is bioactivity acceptable and nontoxic to a subject. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a pharmaceutical composition provided herein decreases oxidation of the polypeptide complex or the bispecific polypeptide complex. This reduction in oxidation prevents or reduces loss of binding affinity, thereby improving protein stability and maximizing shelf-life. Therefore, in certain embodiments, compositions are provided that comprise the polypeptide complex or the bispecific polypeptide complex disclosed herein and one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving the polypeptide complex or the bispecific polypeptide complex as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the polypeptide complex, the bispecific polypeptide complex provided herein or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Method of Treatment

Therapeutic methods are also provided, comprising: administering a therapeutically effective amount of the polypeptide complex or the bispecific polypeptide complex provided herein to a subject in need thereof, thereby treating or preventing a condition or a disorder. In certain embodiments, the subject has been identified as having a disorder or condition likely to respond to the polypeptide complex or the bispecific polypeptide complex provided herein.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The therapeutically effective amount of the polypeptide complex and the bispecific polypeptide complex provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the polypeptide complex or the bispecific polypeptide complex provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the polypeptide complex or the bispecific polypeptide complex provided herein is administered at a dosage of about 50 mg/kg or less, and in certain of these embodiments the dosage is 10 mg/kg or less, 5 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The polypeptide complex or the bispecific polypeptide complex provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In certain embodiments, the condition or disorder treated by the polypeptide complex or the bispecific polypeptide complex provided herein is cancer or a cancerous condition, autoimmune diseases, infectious and parasitic diseases, cardiovascular diseases, neuropathies, neuropsychiatric conditions, injuries, inflammations, or coagulation disorder.

With regard to cancer, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

For example, with regard to the use of the polypeptide complex or bispecific polypeptide complex disclosed herein to treat cancer, a therapeutically effective amount is the dosage or concentration of the polypeptide complex capable of eradicating all or part of a tumor, inhibiting or slowing tumor growth, inhibiting growth or proliferation of cells mediating a cancerous condition, inhibiting tumor cell metastasis, ameliorating any symptom or marker associated with a tumor or cancerous condition, preventing or delaying the development of a tumor or cancerous condition, or some combination thereof.

In certain embodiments, the conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma.

In certain embodiments, the conditions and disorders include a CD19-related disease or condition, such as, B cell lymphoma, optionally Hodgkin lymphoma or non-Hodgkin lymphoma, wherein the non-Hodgkin lymphoma comprises: Diffuse large B-cell lymphoma (DLBCL), Follicular lymphoma, Marginal zone B-cell lymphoma (MZL), Mucosa-Associated Lymphatic Tissue lymphoma (MALT), Small lymphocytic lymphoma (chronic lymphocytic leukemia, CLL), or Mantle cell lymphoma (MCL), Acute Lymphoblastic Leukemia (ALL), or Waldenstrom's Macroglobulinemia (WM).

In certain embodiments, the conditions and disorders include hyperproliferative conditions or infectious diseases that can be treated via regulation of immune responses by CTLA-4 and/or PD-1. Examples of hyperproliferative conditions include, but are not limited to, solid tumors, hematological cancers, soft tissue tumors, and metastatic lesions.

The polypeptide complex or the bispecific polypeptide complex may be administered alone or in combination with one or more additional therapeutic means or agents.

In certain embodiments, when used for treating cancer or tumor or proliferative disease, the polypeptide complex or the bispecific polypeptide complex provided herein may be administered in combination with chemotherapy, radiation therapy, surgery for the treatment of cancer (e.g., tumorectomy), one or more anti-emetics or other treatments for complications arising from chemotherapy, or any other therapeutic agent for use in the treatment of cancer or any medical disorder that related. “Administered in combination” as used herein includes administration simultaneously as part of the same pharmaceutical composition, simultaneously as separate compositions, or at different timings as separate compositions. A composition administered prior to or after another agent is considered to be administered “in combination” with that agent as the phrase is used herein, even if the composition and the second agent are administered via different routes. Where possible, additional therapeutic agents administered in combination with the polypeptide complex or the bispecific polypeptide complex provided herein are administered according to the schedule listed in the product information sheet of the additional therapeutic agent, or according to the Physicians' Desk Reference (Physicians' Desk Reference, 70th Ed (2016)) or protocols well known in the art.

In certain embodiments, the therapeutic agents can induce or boost immune response against cancer. For example, a tumor vaccine can be used to induce immune response to certain tumor or cancer. Cytokine therapy can also be used to enhance tumor antigen presentation to the immune system. Examples of cytokine therapy include, without limitation, interferons such as interferon-α, -β, and -γ, colony stimulating factors such as macrophage-CSF, granulocyte macrophage CSF, and granulocyte-CSF, interleukins such IL-1, IL-1α, IL-2, IL-3, IL-4, IL-5, IL-6, I1-7, IL-8, IL-9, IL-10, IL-11, and IL-12, tumor necrosis factors such as TNF-α and TNF-β. Agents that inactivate immunosuppressive targets can also be used, for example, TGF-beta inhibitors, IL-10 inhibitors, and Fas ligand inhibitors. Another group of agents include those that activate immune responsiveness to tumor or cancer cells, for example, those enhance T cell activation (e.g. agonist of T cell costimulatory molecules such as CTLA-4, ICOS and OX-40), and those enhance dendritic cell function and antigen presentation.

### Kits

The present disclosure further provides kits comprising the polypeptide complex or the bispecific polypeptide complex provided herein. In some embodiments, the kits are useful for detecting the presence or level of, or capturing or enriching one or more target of interest in a biological sample. The biological sample can comprise a cell or a tissue.

In some embodiments, the kit comprises the polypeptide complex or the bispecific polypeptide complex provided herein which is conjugated with a detectable label. In certain other embodiments, the kit comprises an unlabeled polypeptide complex or the bispecific polypeptide complex provided herein, and further comprises a secondary labeled antibody which is capable of binding to the unlabeled polypeptide complex or the bispecific polypeptide complex provided herein. The kit may further comprise an instruction of use, and a package that separates each of the components in the kit.

In certain embodiments, the polypeptide complex or the bispecific polypeptide complex provided herein are associated with a substrate or a device. Useful substrate or device can be, for example, magnetic beads, microtiter plate, or test strip. Such can be useful for a binding assay (such as ELISA), an immunographic assay, capturing or enriching of a target molecule in a biological sample.

The following examples are provided to better illustrate the present disclosure and are not to be interpreted as limiting the scope of the invention. All specific compositions, materials, and methods described below, in whole or in part, fall within the scope of the present disclosure. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present disclosure. It is the intention of the inventors that such variations are included within the scope of the disclosure.

### EXAMPLES

### 1. Methods

### 1.1. Generation of Bispecific Antibodies Exemplified in Tables 1 and 10

Polynucleotides encoding the VL, VH, Ck, and CH1, respectively were amplified by PCR from existing in-house DNA templates. Polynucleotides encoding the CAlpha and CBeta regions were synthesized by Genewiz Inc. Polynucleotides encoding native or chimeric light chain sequences of the antibodies were inserted into a linearized vector containing a CMV promoter and a kappa or lambda signal peptide.

The DNA fragments of Anti target 1 VH-CH1 and Anti target 2 VH-CBeta were inserted into a linearized vector containing human IgG1 or human IgG4 (S228P) constant region CH2-CH3 with a (G4S)n linker according to the formats, e.g., E17R and G25R. For example, the (G4S)n linker can be located between the two binding moieties (e.g., between CH1 of a first binding moiety and VH2 of a second binding moiety) or between the Fc and a binding moiety. In certain embodiments, E17R does not have a (G4S)n linker. In certain embodiments, G25R has a (G4S)n linker between the two binding moieties.

The vector contains a CMV promoter and a human antibody heavy chain signal peptide.

### 1.2. Expression and Purification

Heavy chain and light chain expression plasmids were co-transfected into Expi293 cells using Expi293 expression system kit (ThermoFisher-A14635) according to the manufacturer's instructions. Five days after transfection, the supernatants were collected and the protein was purified using protein A column (GE Healthcare—17543 802). Antibody concentration was measured by Nano Drop. The purity of proteins was evaluated by SDS-PAGE and HPLC-SEC.

### 1.3. Differential Scanning Fluorimetry (DSF)

Melting temperature (Tm) of antibodies was investigated using QuantStudio™ 7 Flex

Real-Time PCR system (Applied Biosystems). 19 μL of antibody solution was mixed with 1 μL of 62.5× SYPRO Orange solution (Invitrogen) and transferred to a 96 well plate (Biosystems). The plate was heated from 26° C. to 95° C. at a rate of 0.9° C./min, and the resulting fluorescence data was collected. The negative derivatives of the fluorescence changes with respect to different temperatures were calculated, Data collection and Tonset/Tm calculation were conducted automatically by the operation Software (QuantStudio™ Real Time PCR software v1.3).

### 1.4. Differential Scanning Calorimetry (DSC)

The DSC analysis was performed by a Malvern DSC System. The protein sample was first diluted to 1 mg/mL with formulation buffer before analysis. 400 μL respective formulation buffer was added to a 96-well plate as reference and 400 μL protein sample was added. The samples were heated from 10° C. to 95° C. at a heating rate of 90° C./h in the DSC system. The DSC results (Tm Onset and Tm values) were analyzed by vendor's software(MicroCal PEAQ DSC Software 1.30 and Malvern).

### 1.5. Tagg-Onset Measurement by Dynamic Light Scattering (DLS)

Tagg-onset measurement was investigated using DynaPro Plate Reader III (Wyatt Dynapro™). 3 acquisitions were collected for each protein sample while each acquisition time was 5 s. Each well contained 7.5 μL of antibody solution and 5 μL of silicone oil in 1536 plate (Aurora microplate). The plate was heated from 40° C. to 80° C. at a rate of 0.125° C./min. For each measurement, the diffusion coefficient was determined and plotted against temperature. Tagg-onset values were calculated automatically by the operation software (DYNAMICS 7.8.1.3).

### 1.6. 40° C. Accelerated Thermostability Test

The antibodies were incubated at 40° C. using Eppendorf Constant temperature mixer, and measure the absorption value of protein solution at 280 nm by Nanodrop 2000, record the appearance and detect the purity by SEC-HPLC after 0 Day, 1 Day, 3 Day, 7 Day, 14 Day, 27 Day and 34 Day.

### 1.7. O-Glycan Mass Spectrum

A 20 μg protein sample (the sample volume was calculated based on the protein concentration) was added to a 1.5 mL tube. The protein sample was made up to 20 μL with 4 μL 5× Rapid PNGase F Buffer, 1 μL 1 mol/L DTT and purified water. The resulting protein sample was mixed well and incubated at 75° C. for 5˜7 min. The sample was then cooled down to room temperature and added 1 μL Rapid PNGase F, mixed well and incubated at 50° C. for 10˜15 min. After incubation, the sample was then added 30 μL purified water and mixed well, resulting in a deglycosylated sample. The deglycosylated sample was then transferred to a HPLC vial with glass-insert vial for analysis.

### 1.8. Binding to Target-Expression Cells

Human PD-L1-expressing cells W315-CHOK1.hPro1.C11 (×105 cells/well) and 4-1BB-expressing cells WBP342-CHO-K1.hPro1.C1 were incubated with various concentrations of testing antibodies at 4° C. for 1 hour. After washing, the secondary antibody R-PE goat anti-human IgG (Jackson-109-115-098) was added and incubated with the cells at 4° C. for 1 hour. The cells were then washed and resuspended in DPBS with 1% BSA. MFI of the cells was measured by a flow cytometer and analyzed by FlowJo.

### 1.9. Reporter Gene Assay

The effector cells WBP342-CHO-K1.hPro1.NFκB.C1D8 and PD-L1-expressing cells W315-CHOK1.hPro1.C11 were harvested, washed and resuspended in F-12K complete medium. Various concentrations of the testing antibodies in complete medium were added to the PD-L1-expressing CHO-K1 cells. Then the RGA effector cells, WBP342-CHO-K1.hPro1.NFκB.C1D8 cells, which stably express full length of human 4-1BB and along with stably integrated NFκB luciferase reporter gene, were added to each well. Human IgG1 isotype antibody was used as negative control. The plates were incubated at 37° C., 5% CO₂ for 4-6 hours. After incubation, reconstituted Nano-Glo luciferase substrate (Promega) was added and the luciferase intensity was measured by a microplate reader.

### 1.10. Rodent Pharmacokinetics (PK)

5 animals as one group were used in this study. Animals were administered with antibodies at 10 mg/kg once in 10 minutes intravenous infusion, respectively. Baseline samples (pre dose) were collected on Study Day-1. PK samples were collected at 0.5 h, 2 h, 6 h, 24 h, 48 h, 72 h, 120 h, 168 h, 240 h, 288 h, 336 h and 504 h after finished dosing in rats.

Antidrug antibody (ADA) samples were collected at pre-dose (Day-1), and post-dose at 168 h and 240 h. Serum concentrations of antibodies and ADA in serum samples were determined by ELISA.

### 2. Results

### 2.1.1. Engineering Designs of T8311 WuXiBody™ Molecules (2+2, Symmetric)

In this exemplary embodiment, the CAlpha region and the CAlpha-CBeta interface region of symmetric 2+2 WuXiBody™ molecules termed as T8311-U14T2.G25R-?.uIgG1 series were engineered. Certain mutations were introduced using Rosetta design. (Froning et al., NATURE COMMUNICATIONS, (2020) 11:2330, https://doi.org/10.1038/s41467-020-16231-7.) The CAlpha sequence (TCR alpha sequence) and CBeta sequence (TCR beta sequence) of the T8311-U14T2.G25R-?.uIgG1 molecules are listed in Table 1. The names of the “T8311-U14T2.G25R-?.uIgG1” series of molecules can be shortened. For example, T8311-U14T2.G25R-57.uIgG1 can be referred to as T8311-57, or design 57.

The full length amino acid sequences of T8311-U14T2.G25R-1.uIgG1 (as1 denoted as T8311-1 in Table 1) are set forth in Table 22. Each of the other antibodies (e.g., T8311-8, T8311-26, T8311-29, etc.) listed in Table 1 comprises the same amino acid sequences as T8311-U14T2.G25R-1.uIgG1 except for the Calpha and CBeta sequences as set forth in Table 1. For example, T8311-1 comprises SEQ ID NO: 10 in the CAlpha region and SEQ ID NO: 11 in the CBeta region and hinge area; T8311-8 comprises SEQ ID NO: 12 in the CAlpha region and SEQ ID NO: 13 in the CBeta region and hinge area. T8311-1 and T8311-8 comprise the same sequences in other regions of the antibodies except for the Calpha and CBeta sequences as shown in Table 1: SEQ ID NO: 10 and 11 (T8311-1) versus SEQ ID NO: 12 and 13 (for T8311-8).

TABLE 1
Design details of WuXiBody ™ symmetric 2 + 2 format

| T8311- | | | |
|---|---|---|---|
| U14T2. | | | |
| G25R-?. | | | |
| uigG1 | TCR alpha sequence | TCR beta sequence | Comments |
| T8311-1 | ...PDIQNPDPAVYQLRDSKSSDK | ...LEDLKNVEPPEVAVFEPSEAE | T8311-U14T2.G25R-1.uIgG1 comprises the heavy chain and light chains set forth in |
| | SVCLETDEDSQTQVSQSKDSDVYI | ISHTQKATLVCLATGFYPDHVELS | Table 22 as SEQ ID NO. 66, 67 and 68, respectively. The amino acid residues of |
| | TDKCVLDMRSMDFKSNSAVAWSQK | WWVNGKEVHSGVCTDPQPLKEQPA | C-terminal (92-95, numbering shown in FIG. 12) in Calpha are bolded. The amino acid |
| | SDFACANAFQNSIIPEDTFFPSPE | LQDSRYALSSRLRVSATFWQNPRN | residues of a hinge region, attached to the C-terminus of the CBeta, are underlined. |
| | SS (SEQ ID NO: 10) | HFRCQVQFYGLSENDEWTQDRAKP | As with the rest of the antibodies in this chart, only the sequence around the |
| | | VTQIVSAEAWGRASDKTHTCPPCP | CAlpha region and the sequence around the C-Beta region of T8311-U14T2.G25R-1.uIgG1 |
| | | ... (SEQ ID NO: 11) | are shown in this chart. |
| T8311-8 | ...PDIQNPDPAVYQLRDSKSSDK | ...LEDLKNVFPPEVAVFEPSKAE | Comprises the original TCR native disulfide bond (C96 on CAlpha (numbering shown in |
| | SVCLFTDFDSQTQVSQSKDSDVYI | IAHTQKATLVCLATGFYPDHVELS | FIG. 12), and A128, D129 and C130 on CBeta (numbering shown in FIG. 13), and |
| | TDKCVLDMRSMDFKSNSAVAWSQK | WWVNGKEVHSGVCTDPQPLKEQPA | mutations to corresponding murine TCR residues (P92S, E93D, S94V, S95P (numbering |
| | SDFACANAFQNSIIPEDTFFPSSD | LQDSRYALSSRLRVSATFWQNPRN | FIG. 12) at the C-terminus of CAlpha, E17K, and S21A (numbering shown in FIG. 13) |
| | VPC (SEQ ID NO: 12) | HFRCQVQFYGLSENDEWTQDRAKP | at CBeta), with different amino acid residues bolded, in comparison to T8311-U14T2. |
| | | VTQIVSAEAWGRADCDKTHTCPPC | G25R-1.uIgG1 |
| | | P... (SEQ ID NO: 13) | |
| T8311-26 | ...PDIQNPDPAVYQLRDSKSSDK | ...LEDLKNVFPPEVAVFEPSEAE | Comprises the original TCR native disulfide bond, amino acid residue “G” at the |
| | SVCLFTDFDSQTQVSQSKDSDVYI | ISHTQKATLVCLATGFYPDHVELS | C-terminal end of CBeta, and mutations at the C-terminus of CAlpha, “VEPKS” in place |
| | TDKCVLDMRSMDFKSNSAVAWSQK | WWVNGKEVHSGVCTDPQPLKEQPA | of “PESS,” with different amino acid residues bolded, in comparison to T8311-U14T2. |
| | SDFACANAFQNSIIPEDTFFPSVE | LQDSRYALSSRLRVSATFWQNPRN | G25R-1.uIgG1. ”EPKS” is derived from a human IgG1 hinge sequence. |
| | PKSC (SEQ ID NO: 14) | HFRCQVQFYGLSENDEWTQDRAKP | |
| | | VTQIVSAEAWGRADCGDKTHTCPP | |
| | | CP... (SEQ ID NO: 15) | |
| T8311-29 | ...PDIQNPDPAVYQLRDSKSSDK | ...LEDLKNVFPPEVAVFEPSEAE | Comprises the original TCR native disulfide bond, amino acid residue “G” at the |
| | SVCLFTDFDSQTQVSQSKDSDVYI | ISHTQKATLVCLATGFYPDHVELS | C-terminal end of CBeta, and mutations at the C-terminus of CAlpha, “NRGE” in place |
| | TDKCVLDMRSMDFKSNSAVAWSQK | WWVNGKEVHSGVCTDPQPLKEQPA | of “PESS,” with different amino acid residues bolded, in comparison to T8311-U14T2. |
| | SDFACANAFQNSIIPEDTFFPSNR | LQDSRYALSSRLRVSATFWQNPRN | G25R-1.uIgG1 |
| | GEC (SEQ ID NO: 16) | HFRCQVQFYGLSENDEWTQDRAKP | |
| | | VTQIVSAEAWGRADCGDKTHTCPP | |
| | | CP... (SEQ ID NO: 17) | |
| T8311-45 | ...PDIQNPDPAVYQLRDSKSSDK | ...LEDLKNVEPPEVAVFEPSEAE | Has deletions of the last eight residues (88-95, numbering shown in FIG. 12) at the |
| | SVCLFTDFDSQTQVSQSKDSDVYI | ISHTQKATLVCLATGFYPDHVELS | c-terminus of CAlpha, in comparison to T8311-U14T2.G25R-1.uIgG1 |
| | TDKCVLDMRSMDFKSNSAVAWSQK | WWVNGKEVHSGVCTDPQPLKEQPA | |
| | SDFACANAFQNSIIPEDT (SEQ | LQDSRYALSSRLRVSATFWQNPRN | |
| | ID NO: 18) | HFRCQVQFYGLSENDEWTQDRAKP | |
| | | VTQIVSAEAWGRASDKTHTCPPCP | |
| | | ... (SEQ ID NO: 19) | |
| T8311-46 | ...PDIQNPDPAVYQLRDSKSSDK | ...LEDLKNVEPPEVAVFEPSEAE | Introduce additional disulfide bond with F26C and F78C at Calpha (numbering shown in |
| | SVCLCTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDEKSNSAVAWSQK | WWVNGKEVHSGVCTDPQPLKEQPA | FIG. 12), with different amino acid residues bolded, in comparison to T8311-U14T2. |
| | SDFACANACQNSIIPEDTFFPS | LQDSRYALSSRLRVSATFWQNPRN | G25R-1.uIgG1 |
| | (SEQ ID NO: 20) | HFRCQVQFYGLSENDEWTQDRAKP | |
| | | VTQIVSAEAWGRASDKTHTCPPCP | |
| | | ... (SEQ ID NO: 21) | |
| T8311-47 | ...PDIQNPDPAVYQLRDSKSSDK | ...LEDLKNVFPPEVAVFEPSEAE | Introduce additional disulfide bond with S36C and N80C at Calpha (numbering shown in |
| | SVCLFTDEDSQTQVCQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12),w ith different amino acid residues bolded, in comparison to T8311-U14T2. |
| | SDFACANAFQCSIIPEDTFFPS | LQDSRYALSSRLRVSATFWQNPRN | G25R-1.uIgG1 |
| | (SEQ ID NO: 22) | HFRCQVQFYGLSENDEWTQDRAKP | |
| | | VTQIVSAEAWGRASDKTHTCPPCP | |
| | | ... (SEQ ID NO: 23) | |
| T8311-48 | ...PDIQNPDPAVYQLRDSKSSDK | ...LEDLKNVFPPEVAVFEPSEAE | Introduce additional disulfide bond with S38C and N80C at Calpha (numbering shown in |
| | SVCLFTDFDSQTQVSQCKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQCSIIPEDTFFPS | LQDSRYALSSRLRVSATFWQNPRN | G25R-1.uIgG1 |
| | (SEQ ID NO: 24) | HFRCQVQFYGLSENDEWTQDRAKP | |
| | | VTQIVSAEAWGRASDKTHTCPPCP | |
| | | ... (SEQ ID NO: 25) | |
| T8311-49 | ...PDIQNPDPAVYQLRDSKSSDK | ...LEDLKNVFPPEVAVFEPSEAE | Introduce additional disulfide bond with K39C and N80C at Calpha (numbering shown in |
| | SVCLFTDFDSQTQVSQSCDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQCSIIPEDTFFPS | LQDSRYALSSRLRVSATFWQNPRN | G25R-1.uIgG1 |
| | (SEQ ID NO: 26) | HFRCQVQFYGLSENDEWTQDRAKP | |
| | | VTQIVSAEAWGRASDKTHTCPPCP | |
| | | ... (SEQ ID NO: 27) | |
| T8311-50 | ...PDIQNPDPAVYQLRDSKSSDK | ...LEDLKNVEPPEVAVFEPSEAE | Introduce additional disulfide bond with Q34C and S81C at Calpha (numbering shown in |
| | SVCLFTDFDSQTCVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNCIIPEDTFFPS | LQDSRYALSSRLRVSATFWQNPRN | G25R-1.uIgG1 |
| | (SEQ ID NO: 28) | HFRCQVQFYGLSENDEWTQDRAKP | |
| | | VTQIVSAEAWGRASDKTHTCPPCP | |
| | | ... (SEQ ID NO: 29) | |
| T8311-51 | ...PDIQNPDPAVYQLRDSKSSDK | ...LEDLKNVFPPEVAVFEPSEAE | Introduce additional disulfide bond with V35C and S81C at Calpha (numbering shown in |
| | SVCLFTDFDSQTQCSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNCIIPEDTFFPS | LQDSRYALSSRLRVSATFWQNPRN | G25R-1.uIgG1 |
| | (SEQ ID NO: 30) | HFRCQVQFYGLSENDEWTQDRAKP | |
| | | VTQIVSAEAWGRASDKTHTCPPCP | |
| | | ... (SEQ ID NO: 31) | |
| T8311-52 | ...PDIQNPDPAVYQLRDSKSSDK | ...LEDLKNVEPPEVAVFEPSEAE | Introduce additional disulfide bond with S36C and S81C at Calpha (numbering shown in |
| | SVCLETDFDSQTQVCQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNCIIPEDTFFPS | LQDSRYALSSRLRVSATFWQNPRN | G25R-1.uIgG1 |
| | (SEQ ID NO: 32) | HFRCQVQFYGLSENDEWTQDRAKP | |
| | | VTQIVSAEAWGRASDKTHTCPPCP | |
| | | ... (SEQ ID NO: 33) | |
| T8311-53 | ...PDIQNPDPAVYQLRDSKSSDK | ...LEDLKNVFPPEVAVFEPSEAE | Introduce additional disulfide bond with Q34C and I82C at Calpha (numbering shown in |
| | SVCLFTDFDSQTCVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSCIPEDTFFPS | LQDSRYALSSRLRVSATFWQNPRN | G25R-1.uIgG1 |
| | (SEQ ID NO: 34) | HFRCQVQFYGLSENDEWTQDRAKP | |
| | | VTQIVSAEAWGRASDKTHTCPPCP | |
| | | ... (SEQ ID NO: 35) | |
| T8311-54 | ...PDIQNPDCAVYQLRDSKSSDK | ...LEDLKNVFPPEVAVFEPSEAE | Introduce additional disulfide bond with P8C and P84C at Calpha (numbering shown in |
| | SVCLFTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIICEDTFFPS | LQDSRYALSSRLRVSATFWQNPRN | G25R-1.uIgG1 |
| | (SEQ ID NO: 36) | HFRCQVQFYGLSENDEWTQDRAKP | |
| | | VTQIVSAEAWGRASDKTHTCPPCT | |
| | | P... (SEQ ID NO: 37) | |
| T8311-55 | ...PDIQNPDPAVYQLRDSKSSDK | ...LEDLKNVFPPEVAVFEPSEAE | Introduce additional disulfide bond with F29C and P84C at Calpha (numbering shown in |
| | SVCLFTDCDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIICEDTFFPS | LQDSRYALSSRLRVSATFWQNPRN | G25R-1.uIgG1 |
| | (SEQ ID NO: 38) | HFRCQVQFYGLSENDEWTQDRAKP | |
| | | VTQIVSAEAWGRASDKTHTCPPCP | |
| | | ... (SEQ ID NO: 39) | |
| T8311-56 | ...PDIQNPDPAVYQLRDSKSSDK | ...LEDLKNVFPPEVAVFEPSEAE | Introduce additional disulfide bond with T33C and P84C at Calpha (numbering shown in |
| | SVCLFTDFDSQCQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIICEDTFFPS | LQDSRYALSSRLRVSATFWQNPRN | G25R-1.uIgG1 |
| | (SEQ ID NO: 40) | HFRCQVQFYGLSENDEWTQDRAKP | |
| | | VTQIVSAEAWGRASDKTHTCPPCP | |
| | | ... (SEQ ID NO: 41) | |
| T8311-57 | ...PDIQNPDCAVYQLRDSKSSDK | ...LEDLKNVFPPEVAVFEPSEAE | Introduce additional disulfide bond with P8C and D86C at Calpha (numbering shown in |
| | SVCLFTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIIPECTFFPS | LQDSRYALSSRLRVSATFWQNPRN | G25R-1.uIgG1 |
| | (SEQ ID NO: 42) | HFRCQVQFYGLSENDEWTQDRAKP | |
| | | VTQIVSAEAWGRASDKTHTCPPCP | |
| | | ... (SEQ ID NO: 43) | |
| T8311-58 | ...PDIQNPDCAVYQLRDSKSSDK | ...LEDLKNVEPPEVAVFEPSEAE | Introduce additional disulfide bond with P8C and T87C at Calpha (numbering shown in |
| | SVCLFTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIIPEDCFFPS | LQDSRYALSSRLRVSATFWQNPRN | G25R-1.uIgG1 |
| | (SEQ ID NO: 44) | HFRCQVQFYGLSENDEWTQDRAKP | |
| | | VTQIVSAEAWGRASDKTHTCPPCP | |
| | | ... (SEQ ID NO: 45) | |
| T8311-59 | ...PDIQNPDPCVYQLRDSKSSDK | ...LEDLKNVFPPEVAVFEPSEAE | Introduce additional disulfide bond with A9C and F88C at Calpha (numbering shown in |
| | SVCLFTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDFKSNSAVAWSQK | WWVNGKEVHSGVCTDPQPLKEQPA | FIG. 12), with different amino acid residues bolded, in comparison to T8311-U14T2. |
| | SDFACANAFQNSIIPEDTCFPS | LQDSRYALSSRLRVSATFWQNPRN | G25R-1.uIgG1 |
| | (SEQ ID NO: 46) | HFRCQVQFYGLSENDEWTQDRAKP | |
| | | VTQIVSAEAWGRASDKTHTCPPCP | |
| | | ... (SEQ ID NO: 47) | |
| T8311-60 | ...PDIQNPDPACYQLRDSKSSDK | ...LEDLKNVEPPEVAVFEPSEAE | Introduce additional disulfide bond with V10C and F89C at Calpha (numbering shown in |
| | SVCLFTDFDSQTQVSQSKDSDVYI | ISHTQKATLVCLATGFYPDHVELS | FIG. 12), delete 4 amino acids at TCR Calpha C terminal (92-95, numbering shown in |
| | TDKCVLDMRSMDEKSNSAVAWSQK | WWVNGKEVHSGVCTDPQPLKEQPA | FIG. 12), with different amino acid residues bolded, in comparison to T8311-U14T2. |
| | SDFACANAFQNSIIPEDTFCPS | LQDSRYALSSRLRVSATFWQNPRN | G25R-1.uIgG1 |
| | (SEQ ID NO: 48) | HFRCQVQFYGLSENDEWTQDRAKP | |
| | | VTQIVSAEAWGRASDKTHTCPPCP | |
| | | ... (SEQ ID NO: 49) | |
| T8311-61 | ...PDIQNPDPAVYQLRDSKSSDK | ...LEDLKNVFPPEVAVFEPSECE | Introduce additional disulfide bond with P90C at Calpha (numbering shown in FIG. 12) |
| | SVCLEDFDSQTQVSQSKDDSDVYI | ISHTQKATLVCLATGFYPDHVELS | and A18C at Cbeta (numbering shown in FIG. 13), delete 4 amino acids at TCR Calpha |
| | TDKCVLDMRSMDFKSNSAVAWSQK | WWVNGKEVHSGVCTDPQPLKEQPA | C terminal (92-95, numbering shown in FIG. 12), with different amino acid residues |
| | SDFACANAFQNSIIPEDTFFCS | LQDSRYALSSRLRVSATFWQNPRN | bolded, in comparison to T8311-U14T2.G25R-1.uIgG1 |
| | | HFRCQVQFYGLSENDEWTQDRAKP | |
| | | VTQIVSAEAWGRASDKTHTCPPCP | |
| | | ... (SEQ ID NO: 51) | |
| T8311-62 | ...PDIQNPDPAVYQLRDSKSSDK | ...LEDLKNVFPPEVAVFEPSEAE | Delete 4 amino acids at TCR Calpha C terminal (92-95, numbering shown in FIG. 12) |
| | SVCLFTDFDSQTQVSQSKDSDVYI | ISHTQKATLVCLATGFYPDHVELS | |
| | TDKCVLDMRSMDFKSNSAVAWSQK | WWVNGKEVHSGVCTDPQPLKEQPA | |
| | SDFACANAFQNSIIPEDTFFPS | LQDSRYALSSRLRVSATFWQNPRN | |
| | (SEQ ID NO: 52) | HFRCQVQFYGLSENDEWTQDRAKP | |
| | | VTQIVSAEAWGRASDKTHTCPPCP | |
| | | ... (SEQ ID NO: 53) | |
| T8311-69 | ...PDIQNPDPAVYQLRDSKSSDK | ...LEDLKNVFPPEVAVFEPSKAE | Introduce 7 mutations from Rosetta design, S22F, T33I, and A73T at Calpha (numbering |
| | FVCLFTDFDSQIQVSQSKDSDVYI | ISRTQKATLVCLATGFYPPHVELS | shown in FIG. 12), and E17K, H22R, D38P, S53D at TCR Cbeta (FIG. 13), with |
| | TDKCVLDMRSMDFKSNSAVAWSQK | WWVNGKEVHDGVCTDPQPLKEQPA | different amino acid residues bolded, in comparison to T8311-U14T2.G25R-1.uIgG1 |
| | SDFTCANAFQNSIIPEDTEFPSPE | LQDSRYALSSRLRVSATFWQNPRN | |
| | SS(SEQ ID NO: 54) | HFRCQVQFYGLSENDEWTQDRAKP | |
| | | VTQIVSAEAWGRASDKTHTCPPCP | |
| | | ... (SEQ ID NO: 55) | |
| T8311-70 | ...PDIQNPDPAVYQLRDSKSSDK | ...LEDLKNVEPPEVAVFEPSKAE | Introduce 7 mutations from Rosetta design, S22F, T33I, and A73T at Calpha (numbering |
| | FVCLFTDFDSQIQVSQSKDSDVYI | ISRTQKATLVCLATGFYPPHVELS | shown in FIG. 12), and E17K, H22R, D38P, and S53D at TCR Cbeta (numbering shown |
| | TDKCVLDMRSMDFKSNSAVAWSQK | WWVNGKEVHDGVCTDPQPLKEQPA | in FIG. 13), comprises the original TCR native disulfide bond (C96 on CAlpha |
| | SDFTCANAFQNSIIPEDTFFPSPE | LQDSRYALSSRLRVSATFWQNPRN | (numbering shown in FIG. 12) and A128, D129 and C130 on CBeta (numbering shown in |
| | SSC (SEQ ID NO: 56) | HFRCQVQFYGLSENDEWTQDRAKP | FIG. 13)), with different amino acid residues bolded, in comparison to T8311- |
| | | VTQIVSAEAWGRADCDKTHTCPPC | U14T2.G25R-1.uIgG1 |
| | | P... (SEQ ID NO: 57) | |
| T8311-71 | ...PDIQNPDCAVYQLRDSKSSDK | ...LEDLKNVFPPEVAVFEPSECE | Introduce additional disulfide bonds with P8C, D86C, and P90C at Calpha (numbering |
| | SVCLETDEDSQTQVSQSKDSDVYI | ISHTQKATLVCLATGFYPDHVELS | shown in FIG. 12), and A18C at Cheta (numbering shown in FIG. 13), delete 4 amino |
| | TDKCVLDMRSMDFKSNSAVAWSQK | WWVNGKEVHSGVCTDPQPLKEQPA | acids at TCR Calpha C terminal (92-95, numbering shown in FIG. 12), with different |
| | SDFACANAFQNSIIPECTFFCS | LQDSRYALSSRLRVSATFWQNPRN | amino acid residues bolded, in comparison to T8311-U14T2.G25R-1.uIgG1 |
| | (SEQ ID NO: 58) | HFRCQVQFYGLSENDEWTQDRAKP | |
| | | VTQIVSAEAWGRASDKTHTCPPCP | |
| | | ... (SEQ ID NO: 59) | |
| T8311-72 | ...PDIQNPDCAVYQLRDSKSSDK | ...LEDLKNVFPPEVAVFEPSEAE | Introduce additional disulfide bond with P8C and D86C at Calpha (numbering shown in |
| | SVCLFTDFDSQTQVSQSKDSDVYI | ISHTQKATLVCLATGFYPDHVELS | FIG. 12), comprises the original TCR native disulfide bond (C96 on CAlpha (numbering |
| | TDKCVLDMRSMDFKSNSAVAWSQK | WWVNGKEVHSGVCTDPQPLKEQPA | shown in FIG. 12) and A128, D129 and C130 on CBeta numbering shown in (FIG. 13)), |
| | SDFACANAFQNSIIPECTFFPSPE | LQDSRYALSSRLRVSATFWQNPRN | with different amino acid residues bolded, in comparison to T8311-U14T2.G25R-1.uIgG1 |
| | SSC (SEQ ID NO: 60) | HFRCQVQFYGLSENDEWTQDRAKP | |
| | | VTQIVSAEAWGRADCDKTHTCPPC | |
| | | P... (SEQ ID NO: 61) | |
| T8311-73 | ...PDIQNPDPAVYQLRDSKSSDK | ...LEDLKNVEPPEVAVFEPSECE | Introduce additional disulfide bond with P90C at Calpha (numbering shown in FIG. 12) |
| | SVCLFTDFDSQTQVSQSKDSDVYI | ISHTQKATLVCLATGFYPDHVELS | and A18C at Cbeta (numbering shown in FIG. 13), comprises the original TCR native |
| | TDKCVLDMRSMDFKSNSAVAWSQK | WWVNGKEVHSGVCTDPQPLKEQPA | disulfide bond (C96 on CAlpha (numbering shown in FIG. 12) and A128, D129 and C130 |
| | SDFACANAFQNSIIPEDTFFCSPE | LQDSRYALSSRLRVSATFWQNPRN | on CBeta (numbering shown in FIG. 13)), with different amino acid residues bolded, |
| | SSC (SEQ ID NO: 62) | HFRCQVQFYGLSENDEWTQDRAKP | in comparison to T8311-U14T2.G25R-1.uIgG1 |
| | | VTQIVSAEAWGRADCDKTHTCPPC | |
| | | P... (SEQ ID NO: 63) | |
| T8311-74 | ...PDIQNPDCAVYQLRDSKSSDK | ...LEDLKNVEPPEVAVFEPSECE | Introduce additional disulfide bonds with P8C, D86C, and P90C at Calpha (numbering |
| | SVCLFTDFDSQTQVSQSKDSDVYI | ISHTQKATLVCLATGFYPDHVELS | shown in FIG. 12), and A18C at Cbeta (numbering shown in FIG. 13), comprises the |
| | TDKCVLDMRSMDFKSNSAVAWSQK | WWVNGKEVHSGVCTDPQPLKEQPA | original TCR native disulfide bond (C96 on CAlpha (numbering shown in FIG. 12) and |
| | SDFACANAFQNSIIPECTEFCSPE | LQDSRYALSSRLRVSATFWQNPRN | A128, D129 and C130 on CBeta (numbering shown in FIG. 13)) with different amino |
| | SSC (SEQ ID NO: 64) | HFRCQVQFYGLSENDEWTQDRAKP | acid residues bolded, in comparison to T8311-U14T2.G25R-1.uIgG1 |
| | | VTQIVSAEAWGRADCDKTHTCPPC | |
| | | P... (SEQ ID NO: 65) | |
| T8311-78 | ...PDIQNPDCAVYQLRDSKSSDK | ...LEDLKNVFPPEVAVFEPSEAE | Introduce additional disulfide bond with P8C and D86C at Calpha (numbering shown in |
| | SVCLFTDFDSQTQVSQSKDSDVYI | ISHTQKATLVCLATGFYPDHVELS | FIG. 12), with different amino acid residues bolded, in comparison to W329001-U4T4. |
| | TDKCVLDMRSMDFKSNSAVAWSQK | WWVNGKEVHSGVCTDPQPLKEQPA | G25R-1.uIgG1 |
| | SDFACANAFQNSIIPECTFFPSPE | LQDSRYALSSRLRVSATFWQNPRN | |
| | SS (SEQ ID NO: 79) | HFRCQVQFYGLSENDEWTQDRAKP | |
| | | VTQIVSAEAWGRASDKTHTCPPCP | |
| | | ... (SEQ ID NO: 43) | |
| T8311-79 | ...PDIQNPDPAVYQLRDSKSSDK | ...LEDLKNVEPPEVAVFEPSECE | Introduce additional disulfide bond with P90C at Calpha (numbering shown in FIG. 12) |
| | SVCLFTDEDSQTQVSQSKDSDVYI | ISHTQKATLVCLATGFYPDHVELS | and A18C at Cbeta (numbering shown in FIG. 13);with different amino acid residues |
| | TDKCVLDMRSMDFKSNSAVAWSQK | WWVNGKEVHSGVCTDPQPLKEQPA | bolded, in comparison to W329001-U4T4.G25R-1.uIgG1 |
| | SDFACANAFQNSIIPEDTFFCS | LQDSRYALSSRLRVSATFWQNPRN | |
| | PESS (SEQ ID NO: 80) | HFRCQVQFYGLSENDEWTQDRAKP | |
| | | VTQIVSAEAWGRASDKTHTCPPCP | |
| | | ... (SEQ ID NO: 51) | |

### 2.1.2. Protein Production of T8311 WuXiBody™ Molecules (2+2, Symmetric)

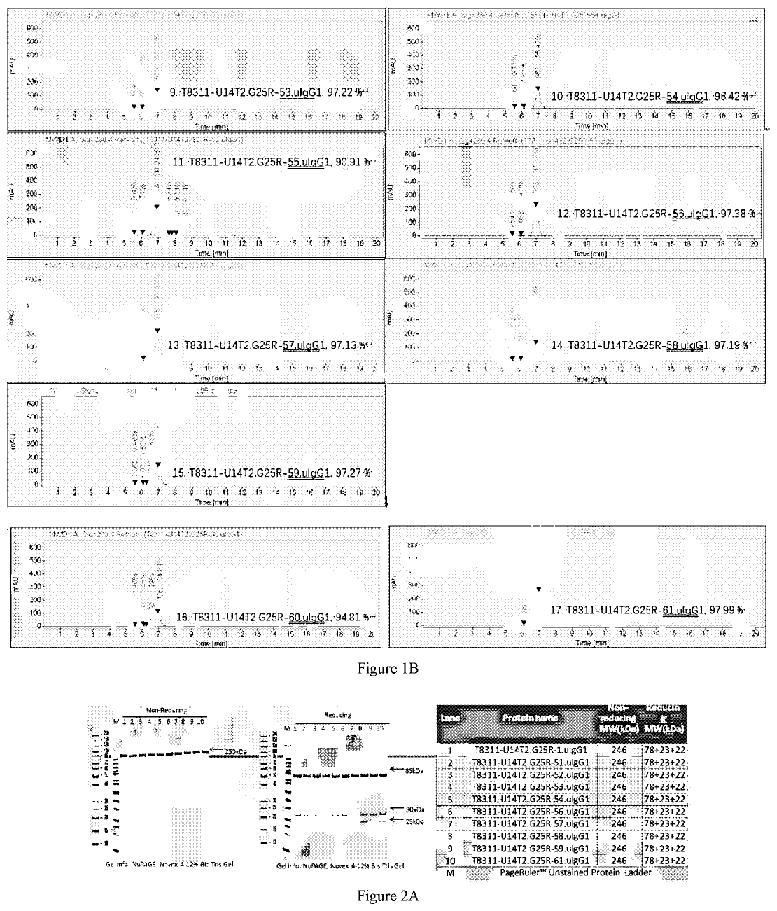

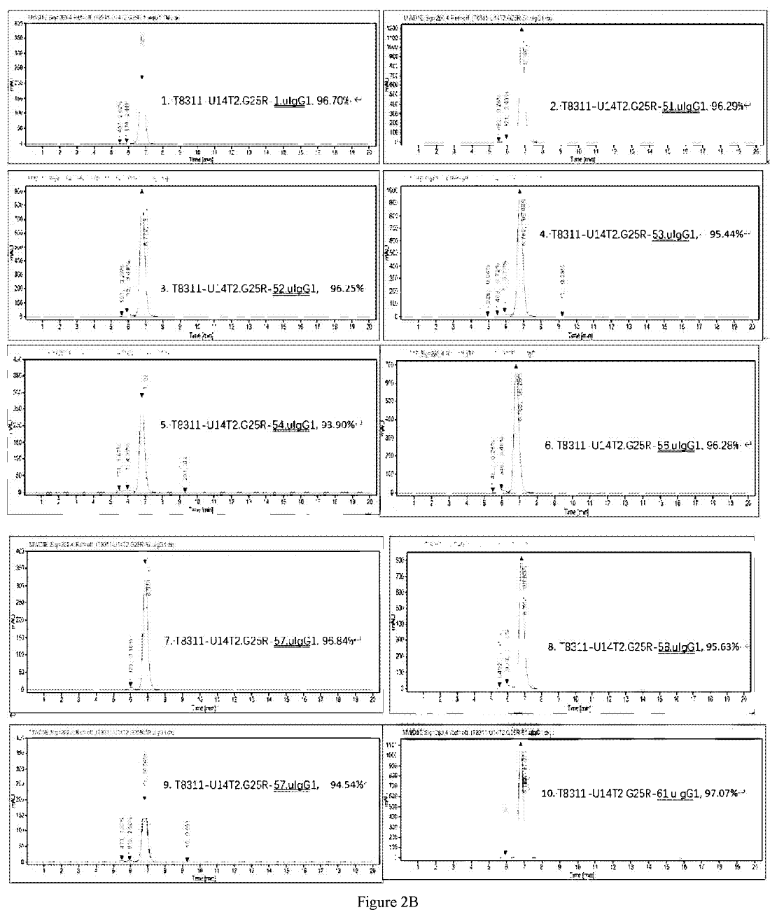

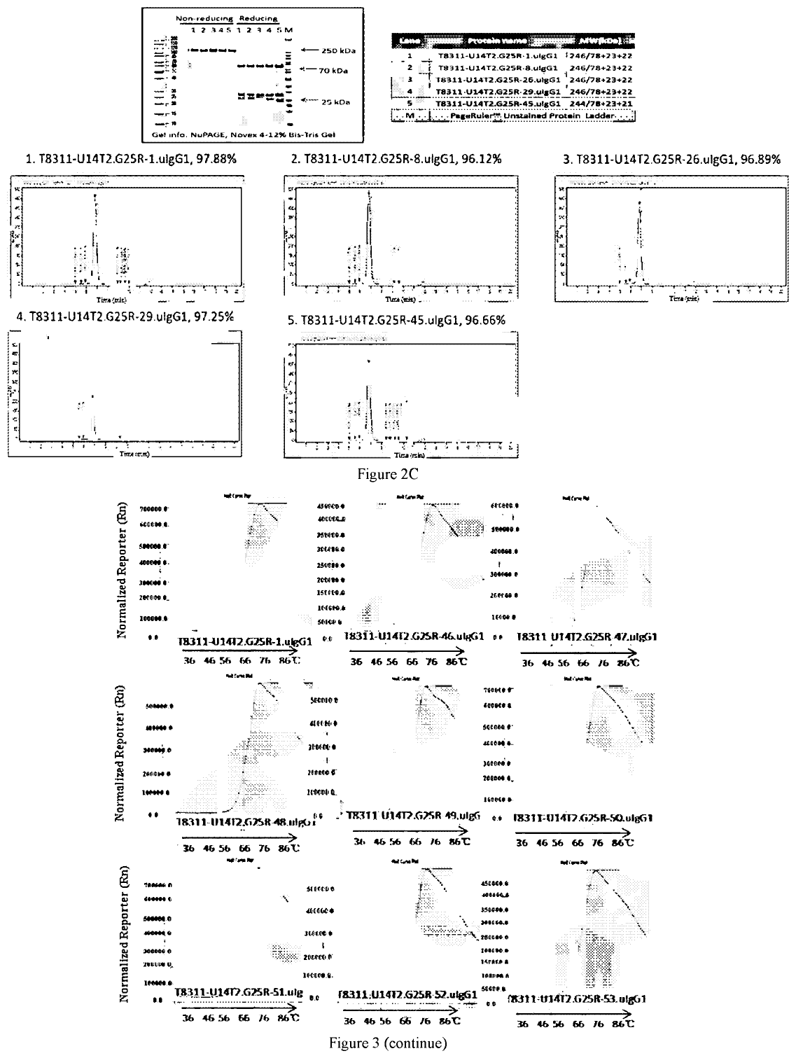

Table 2 summarizes the yield and purity of the proteins including T8311-U14T2.G25R-1.uIgG1, T8311-U14T2.G25R-8.uIgG1, T8311-U14T2.G25R-26.uIgG1, T8311-U14T2.G25R-29.uIgG1, T8311-U14T2.G25R-45.uIgG1 to T8311-U14T2.G25R-61.uIgG1 through transient expression in Expi293. After one-step protein A purification, the purities of all the bsAbs reached >90%. As shown in Table 2, T8311-U14T2.G25R-57.uIgG1 and T8311-U14T2.G25R-61.uIgG1 improved the expression level (e.g., yield after one-step purification) by 46-86% and 12-39%, respectively. FIG. 1 and FIG. 2 show SDS-PAGE and SEC-HPLC characterizations of the two batches of the proteins after purification. These bsAbs migrated with the apparent molecular mass of 250 kDa under non-reducing condition, and 75 kDa, 25 kDa under reducing conditions, indicating the intact and well-assembled bispecific molecules.

TABLE 2
Summary of protein production of the T8311 molecules.

| | | | First Batch | | | Second Batch | | |
|---|---|---|---|---|---|---|---|---|
| | | | | | Purity | | | Purity |
| | | | Concentration | Yield | by SEC- | Concentration | Yield | by SEC- |
| Protein Name | PI | Buffer | (mg/ml) | (mg/L) | HPLC (%) | (mg/ml) | (mg/L) | HPLC (%) |
| T8311-U14T2.G25R-1.uIgG1 | 6.12 | PBS | 1.12 | 56.02 | 97.00% | 1.41 | 59.14 | 96.70% |
| T8311-U14T2.G25R-8.uIgG1 | 6.2 | | | | | 1.98 | 32.44 | 96.12% |
| T8311-U14T2.G25R-26.uIgG1 | 6.12 | | | | | 1.6 | 32.04 | 96.89% |
| T8311-U14T2.G25R-29.uIgG1 | 6.12 | | | | | 0.84 | 24.21 | 97.25% |
| T8311-U14T2.G25R-45.uIgG1 | 6.2 | | | | | 1.67 | 46.05 | 96.66% |
| T8311-U14T2.G25R-46.uIgG1 | 6.19 | | 0.68 | 25.46 | 92.12% | | | |
| T8311-U14T2.G25R-47.uIgG1 | 6.19 | | 1.12 | 53.20 | 97.98% | | | |
| T8311-U14T2.G25R-48.uIgG1 | 6.19 | | 1.11 | 55.47 | 98.17% | | | |
| T8311-U14T2.G25R-49.uIgG1 | 6.12 | | 0.94 | 35.43 | 97.66% | | | |
| T8311-U14T2.G25R-50.uIgG1 | 6.19 | | 1.31 | 62.43 | 97.31% | | | |
| T8311-U14T2.G25R-51.uIgG1 | 6.19 | | 2.21 | 71.95 | 97.20% | 4.95 | 79.22 | 96.29% |
| T8311-U14T2.G25R-52.uIgG1 | 6.19 | | 1.56 | 74.06 | 97.36% | 3.67 | 65.99 | 96.25% |
| T8311-U14T2.G25R-53.uIgG1 | 6.19 | | 0.61 | 71.81 | 97.22% | 4.29 | 53.64 | 95.44% |
| T8311-U14T2.G25R-54.uIgG1 | 6.19 | | 0.65 | 32.29 | 96.42% | 1.47 | 40.46 | 93.90% |
| T8311-U14T2.G25R-55.uIgG1 | 6.19 | | 1.05 | 26.16 | 90.91% | | | |
| T8311-U14T2.G25R-56.uIgG1 | 6.19 | | 0.92 | 64.74 | 97.38% | 2.85 | 58.41 | 96.28% |
| T8311-U14T2.G25R-57.uIgG1 | 6.27 | | 0.82 | 104.42 | 97.13% | 1.52 | 86.29 | 96.84% |
| T8311-U14T2.G25R-58.uIgG1 | 6.19 | | 0.58 | 64.80 | 97.19% | 3.37 | 43.75 | 95.63% |
| T8311-U14T2.G25R-59.uIgG1 | 6.19 | | 0.66 | 44.52 | 97.27% | 1.17 | 41.03 | 94.54% |
| T8311-U14T2.G25R-60.uIgG1 | 6.19 | | 0.57 | 39.85 | 94.81% | | | |
| T8311-U14T2.G25R-61.uIgG1 | 6.19 | | 0.91 | 77.67 | 97.99% | 4.08 | 66.27 | 97.07% |

### 2.1.3. DSF Characterizations of T8311 WuXiBody™ Molecules (2+2, Symmetric)

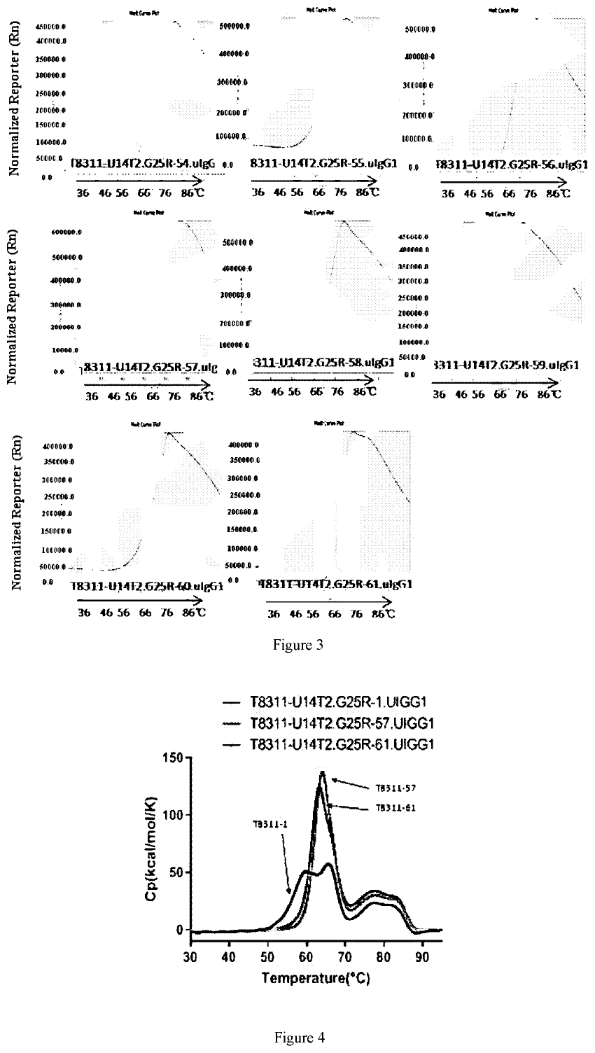

The thermostability of the purified proteins of the T8311 series were characterized by DSF, and the results were listed in Table 3. Most of the T8311 series had more or less improved Tm-onset and Tm1, especially T8311-U14T2.G25R-57.uIgG1 and T8311-U14T2.G25R-61.uIgG1. Compared to reference WuXiBody™ molecule (T8311-U14T2.G25R-1.uIgG1), T8311-U14T2.G25R-57.uIgG1 and T8311-U14T2.G25R-61.uIgG1 improved Tm-onset by 6-8° C., and improved Tm1 by 3° C. FIG. 3 shows the DSF profiles of the first batch of T8311 WuXiBody™ molecules upon temperature increase.

TABLE 3
Summary of DSF characterizations of the T8311 molecules.

| | | | First Batch | | |
|---|---|---|---|---|---|
| | | | Concen- | | |
| | | | tration | Ton | Tm1 |
| Protein Name | pI | Buffer | (mg/ml) | (° C.) | (° C.) |
| T8311-U14T2.G25R-1.uIgG1 | 6.1 | PBS | 1.1 | 50 | 59.2 |
| T8311-U14T2.G25R-46.uIgG1 | 6.2 | | 0.7 | 48 | 57.9 |
| T8311-U14T2.G25R-47.uIgG1 | 6.2 | | 1.1 | 54 | 60.3 |
| T8311-U14T2.G25R-48.uIgG1 | 6.2 | | 1.1 | 54 | 60.5 |
| T8311-U14T2.G25R-49.uIgG1 | 6.1 | | 0.9 | 52 | 58.7 |
| T8311-U14T2.G25R-50.uIgG1 | 6.2 | | 1.3 | 54 | 60.8 |
| T8311-U14T2.G25R-51.uIgG1 | 6.2 | | 2.2 | 54 | 60.2 |
| T8311-U14T2.G25R-52.uIgG1 | 6.2 | | 1.6 | 54 | 60.2 |
| T8311-U14T2.G25R-53.uIgG1 | 6.2 | | 0.6 | 52 | 62.9 |
| T8311-U14T2.G25R-54.uIgG1 | 6.2 | | 0.7 | 54 | 63.4 |
| T8311-U14T2.G25R-55.uIgG1 | 6.2 | | 1.1 | 48 | 57.5 |
| T8311-U14T2.G25R-56.uIgG1 | 6.2 | | 0.9 | 54 | 61.3 |
| T8311-U14T2.G25R-57.uIgG1 | 6.3 | | 0.8 | 56 | 62.4 |
| T8311-U14T2.G25R-58.uIgG1 | 6.2 | | 0.6 | 54 | 59.7 |
| T8311-U14T2.G25R-59.uIgG1 | 6.2 | | 0.7 | 50 | 63.8 |
| T8311-U14T2.G25R-60.uIgG1 | 6.2 | | 0.6 | 48 | 57.7 |
| T8311-U14T2.G25R-61.uIgG1 | 6.2 | | 0.9 | 58 | 62.3 |
| Human-101 | | | | | |

### 2.1.4. DSC Characterization of T8311 WuXiBody™ Molecules (2+2, Symmetric)

The reference T831 1 2+2 molecule (T8311-U14T2.G25R-1.uIgG1), as well as some of the other T8311 molecules were further characterized using DSC. FIG. 4 shows that the T8311 molecules had DSC curves shifted to the right, indicating they had relatively stronger resistance to temperature increase. Table 4 listed the values of Tm-onset and Tm1. T8311-U14T2.G25R-57.uIgG1 and T8311-U14T2.G25R-61.uIgG1 were the two best-performed molecules. U14T2.G25R-57.uIgG1 increased Tm-onset by 8.8° C. and improved Tm1 by 3.6° C. to 64.0° C., which is close to the Tm1 of a regular IgG4 antibody (˜65° C.). T8311-U14T2.G25R-61.uIgG1 also significantly improved Tm-onset and Tm1 by 5.5° C. and 2.8° C., respectively.

TABLE 4
Summary of DSC characterizations for
the designs based on T8311 molecule.

| | Tm- | | | | |
|---|---|---|---|---|---|
| | onset | Tm1 | Tm2 | Tm3 | Tm4 |
| File Name | (° C.) | (° C.) | (° C.) | (° C.) | (° C.) |
| T8311-U14T2.G25R-1.UIGG1[4] | 48.0 | 60.4 | 65.6 | 77.8 | 81.5 |
| T8311-U14T2.G25R-57.UIGG1[16] | 56.8 | 64.0 | 67.4 | 77.1 | 83.2 |
| T8311-U14T2.G25R-61.UIGG1[22] | 53.5 | 63.2 | 67.1 | 76.9 | 83.3 |

### 2.1.5. DLS Characterization of Designs on T8311 WuXiBody™ Molecule (2+2, Symmetric)

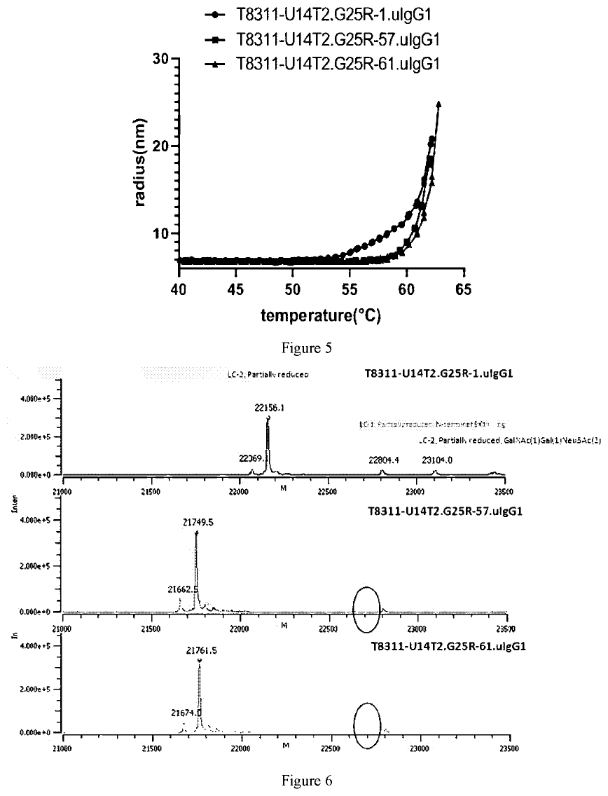

Some T8311 WuXiBody™ molecules were also characterized by DLS as shown in FIG. 5. The obtained Tagg-onset values are listed in Table 5. T8311-U14T2.G25R-57.uIgG1 and T8311-U14T2.G25R-61.uIgG1 ranked as the top 2 variants in terms of the Tagg-onset values. Both designs improved Tagg-onset by about 3-4° C. compared to the reference molecule (T8311-U14T2.G25R-1.uIgG1).

TABLE 5
The Tagg-onset values of T8311 molecules characterized by DLS.

| | | | Concen- | Tagg- |
|---|---|---|---|---|
| | | | tration | onset |
| Protein Name | pI | Buffer | (mg/ml) | (° C.) |
| T8311-U14T2.G25R-1.uIgG1 | 6.1 | PBS | 1.0 | 56.1 |
| T8311-U14T2.G25R-57.uIgG1 | 6.3 | PBS | 1.0 | 59.7 |
| T8311-U14T2.G25R-61.uIgG1 | 6.2 | PBS | 1.0 | 60.0 |

### 2.1.6. 40° C. Accelerated Thermostability Test of T8311 WuXiBody™ Molecules (2+2, Symmetric)

T8311-U14T2.G25R-1.uIgG1, T8311-U14T2.G25R-57.uIgG1 and T8311-U14T2.G25R-61.uIgG1 were further inspected in thermo-stressed condition. The molecules prepared at 5 mg/ml and >90% purity were incubated at 40° C. for two weeks. The purities of these samples were monitored at Day 1, Day 3, Day 7, and Day 14. Table 6 listed the purity changes of the two antibodies.

The purity of the reference antibody (T8311-U14T2.G25R-1.uIgG1) at Day-14 has dropped to the value below 90%, while the purity of T8311-U14T2.G25R-57.uIgG1 (the design of the CAlpha and CBeta sequences of Design-57) and T8311-U14T2.G25R-61.uIgG1 (Design-61) on Day-14 remained above 90%, showing that the two designs also play important role in improving the long-term thermal stability of antibodies. The design (sequences) of the CAlpha and CBeta sequences T8311-U14T2.G25R-57.uIgG1 is termed Design-57 and the design (sequences) of the CAlpha and CBeta sequences T8311-U14T2.G25R-61.uIgG1 is termed Design-61. Further analysis of these data revealed that Design-57 and Design-61 mainly increased the long-term thermal stability of the antibodies by reducing the growth of the aggregation fraction, which is also consistent with the results of DLS.

TABLE 6
Concentration and purity changes of T8311 molecules. All samples were
in an buffer: 20 mM His, 70 mM Trehalose, 0.01% PS-80, 0.2M Arg pH 7.0

| | | T8311-U14T2.G25R- | | | T8311-U14T2.G25R- | | | T8311-U14T2.G25R- | | |
|---|---|---|---|---|---|---|---|---|---|---|
| | | 1.uIgG1 | | | 57.uIgG1 | | | 61.uIgG1 | | |
| Con.(mg/ml) | D 0 | 5.03 | | | 5.08 | | | 5.12 | | |
| | D 1 | 5.02 | | | 5.04 | | | 5.08 | | |
| | D 4 | 5.05 | | | 5.16 | | | 5.14 | | |
| | D 7 | 5.06 | | | 5.34 | | | 5.17 | | |
| | D 14 | 5.30 | | | 6.20 | | | 5.34 | | |
| Appearance | D 0 | CL; PF | | | CL; PF | | | CL; PF | | |
| | D 1 | CL; PF | | | CL; PF | | | CL; PF | | |
| | D 4 | CL; PF | | | CL; PF | | | CL; PF | | |
| | D 7 | CL; PF | | | CL; PF | | | CL; PF | | |
| | D 14 | CL; PF | | | CL; PF | | | CL; PF | | |
| | | Aggregate | Monomer | Fragment | Aggregate | Monomer | Fragment | Aggregate | Monomer | Fragment |
| | — | % | % | % | % | % | % | % | % | % |
| SEC-HPLC | D 0 | 2.25 | 97.73 | 0.04 | 3.40 | 96.58 | 0.02 | 1.29 | 98.66 | 0.04 |
| | D 1 | 4.91 | 95.03 | 0.06 | 3.91 | 94.77 | 1.32 | 1.62 | 98.25 | 0.13 |
| | D 4 | 5.75 | 93.54 | 0.72 | 3.66 | 94.38 | 1.86 | 1.74 | 97.26 | 1.01 |
| | D 7 | 7.48 | 91.26 | 1.26 | 3.98 | 93.14 | 2.88 | 2.08 | 96.46 | 1.46 |
| | D 14 | 9.31 | 86.61 | 4.08 | 3.97 | 90.14 | 5.88 | 2.55 | 94.12 | 3.33 |
| Decrease | | | −11.12 | | | −6.44 | | | −4.54 | |

### 2.1.7. O-Glycan Characterized by Mass Spectrum

The glycosylation conditions of the selected WuXiBody™ molecules were inspected by mass spectroscopy. The reference WuXiBody™ molecule is known to have an O-glycosylation site. In the reducing mass spectrum, the mass of core-1 structured O-glycan (GlcNAc+Hex+2*NeuAc) was observed on the VL-CAlpha light chain of the reference molecule. The O-glycan signal, however, was not observed on the VL-CAlpha chain of Design-57 and Design-61 (FIG. 6).

### 2.1.8. Binding to Target-Expression Cells

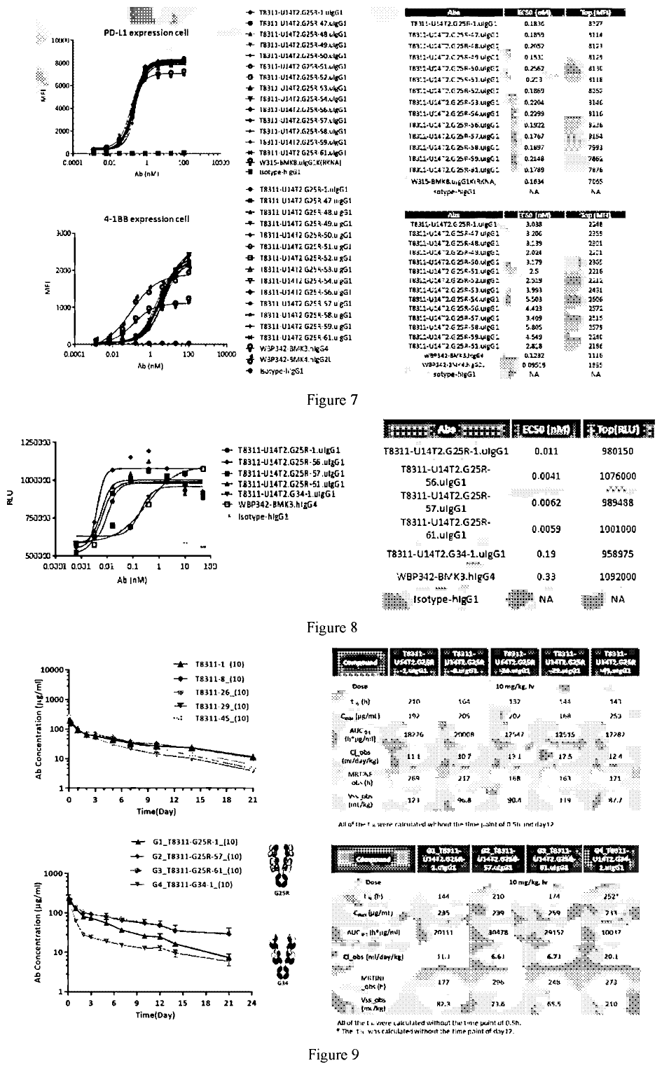

To confirm that these mutations in the CAlpha and/or CBeta regions did not affect the binding capability of the T8311 bsAb, FACS bindings on engineering cells expressing each target (antigen) were carried out. As shown in FIG. 7 and Table 7, compared to the reference molecule, the T8311 variants kept the target binding ability very well.

TABLE 7
EC50 and top MFI values of the FACS binding
of T8311 WuXiBody ™ bsAb and
its designed variants to each target.

| | PD-L1 | | 4-1BB | |
|---|---|---|---|---|
| | EC50 | Top | EC50 | Top |
| Abs | (nM) | (MFI) | (nM) | (MFI) |
| T8311-U14T2.G25R-1.uIgG1 | 0.1836 | 8327 | 3.038 | 2248 |
| T8311-U14T2.G25R-47.uIgG1 | 0.1859 | 8114 | 3.206 | 2359 |
| T8311-U14T2.G25R-48.uIgG1 | 0.2052 | 8123 | 3.139 | 2301 |
| T8311-U14T2.G25R-49.uIgG1 | 0.1531 | 8129 | 2.024 | 2201 |
| T8311-U14T2.G25R-50.uIgG1 | 0.2562 | 8130 | 3.179 | 2365 |
| T8311-U14T2.G25R-51.uIgG1 | 0.213 | 8118 | 2.5 | 2216 |
| T8311-U14T2.G25R-52.uIgG1 | 0.1869 | 8062 | 2.519 | 2212 |
| T8311-U14T2.G25R-53.uIgG1 | 0.2204 | 8146 | 3.993 | 2431 |
| T8311-U14T2.G25R-54.uIgG1 | 0.2299 | 8116 | 5.503 | 2606 |
| T8311-U14T2.G25R-56.uIgG1 | 0.1922 | 8236 | 4.423 | 2572 |
| T8311-U14T2.G25R-57.uIgG1 | 0.1767 | 8154 | 3.409 | 2515 |
| T8311-U14T2.G25R-58.uIgG1 | 0.1897 | 7993 | 5.805 | 2579 |
| T8311-U14T2.G25R-59.uIgG1 | 0.2148 | 7862 | 4.549 | 2240 |
| T8311-U14T2.G25R-61.uIgG1 | 0.1789 | 7876 | 2.818 | 2186 |
| W315-BMK8.uIgGIK(RKNA) | 0.1634 | 7065 | | |
| WBP342-BMK3.hIgG4 | | | 0.1232 | 1116 |
| WBP342-BMK4.hIgG2L | | | 0.09519 | 1895 |
| Isotype-hIgG1 | NA | NA | NA | NA |

FIG. 7 Shows FACS binding of T8311 WuXiBody™ bsAbs to two targets. Thicker curves were the positive control to each target. EC50 and top MFI values were listed in Table 7.

### 2.1.9. Reporter Gene Assay

The function of T8311 WuXiBody™ bsAbs were checked in a reporter gene assay. When cross-linked by target-A expressing cells, T8311-U14T2.G25R-1.uIgG1, T8311-U14T2.G25R-57.uIgG1 and T8311-U14T2.G25R-61.uIgG1 showed comparable agonist effect in activating target-B mediated NF-KB pathway. Data were shown in Table 8 and FIG. 8.

TABLE 8
Detailed results (EC50 and top) of reporter gene assay

| | EC50 | Top |
|---|---|---|
| Abs | (nM) | (RLU) |
| T8311-U14T2.G25R-1.uIgG1 | 0.011 | 980150 |
| T8311-U14T2.G25R-56.uIgG1 | 0.0041 | 1076000 |
| T8311-U14T2.G25R-57.uIgG1 | 0.0062 | 989488 |
| T8311-U14T2.G25R-61.uIgG1 | 0.0059 | 1001000 |
| WBP342-BMK3.hIgG4 | 0.33 | 1092000 |
| Isotype-hIgG1 | NA | NA |

FIG. 8 shows the Reporter gene assay results to check the function of T8311 bsAbs.

### 2.1.10. Rodent PK

The PK of the designed WuXiBody™ molecules were evaluated in rats. SD rats were i.v. administrated a single dose at 10 mg/kg. Plasma concentrations of the molecules were measured by ELISA method. The samples were collected for 21 days. To make a side-by-side comparison, we also built a control format G34 using scFab. As shown in Table 9A, Design-8, Design-26, Design-29, and Design-45 showed comparable drug exposure and PK parameters compared to the reference (T8311-U14T2.G25R-1.uIgG1). The G34 format is shown in FIG. 14, which comprises scFab, but not Calpha and Cbeta. As shown in Table 9B, Design-57 and Design-61 had significantly improved drug exposure. Compared to the clearance of 11.1 ml/day/kg of the reference molecule, the clearance of T8311-U14T2.G25R-57.uIgG1 and T8311-U14T2.G25R-61.uIgG1 was reduced to 6.6 ml/day/kg and 6.7 ml/day/kg, respectively (Table 9B). This was a 40% improvement, and achieved a level close to a regular monoclonal antibody. The control format G34, however, showed the worst PK behaviors.

TABLE 9A
PK results of T8311 WuXiBody ™ molecules

| | T8311- | T8311- | T8311- | T8311- | T8311- |
|---|---|---|---|---|---|
| | U14T2.G25R- | U14T2.G25R- | U14T2.G25R- | U14T2.G25R- | U14T2.G25R- |
| Compound | 1.uIgG1 | 8.uIgG1 | 26.uIgG1 | 29.uIgG1 | 45.uIgG1 |
| Dose | 10 mg/kg, iv | | | | |
| t½ (h) | 210 | 164 | 132 | 144 | 143 |
| Cmax (μ/mL) | 192 | 205 | 202 | 168 | 253 |
| AUC 0-t (h*μg/ml) | 18276 | 20008 | 17547 | 12515 | 17282 |
| Cl_obs (ml/day/kg) | 11.1 | 10.7 | 13.1 | 17.5 | 12.4 |
| MRTINF(obs (h)) | 269 | 217 | 168 | 163 | 171 |
| Vss_obs((mL/kg)) | 123 | 96.8 | 90.4 | 119 | 87.7 |

TABLE 9B
PK results of T8311 WuXiBody ™ molecules

| | G1_T8311- | G2_T8311- | G3_T8311- | G4_T8311- |
|---|---|---|---|---|
| | U14T2.G25R- | U14T2.G25R- | U14T2.G25R- | U14T2.G34- |
| Compound | 1.uIgG1 | 57.uIgG1 | 61.uIgG1 | 1.uIgG1 |
| Dose | 10 mg/kg, iv | | | |
| t_{1/2} (h) | 144 | 210 | 174 | 252 |
| Cₘₐₓ (μg/mL) | 235 | 239 | 259 | 233 |
| AUC₀₋ₜ (h*μg/ml) | 20111 | 30478 | 29152 | 10037 |
| Cl_obs (ml/day/kg) | 11.1 | 6.61 | 6.71 | 20.1 |
| MRTINF_obs (h) | 177 | 296 | 248 | 273 |
| Vss_obs (mL/kg) | 82.3 | 73.6 | 65.5 | 210 |

### 2.2.1. WuXiBody™ Engineering on W3618 Molecules (1+1, Asymmetric)

TABLE 10
Design details in W3618 asymmetric 1 + 1 format WuXiBody ™ molecules

| W3618- | | | |
|---|---|---|---|
| U4T1. | | | |
| E17R-?. | | | |
| uIgG1 | TCR alpha sequence | TCR beta sequence | Comments |
| W3618- | ...PDIQNPDPAVYQLRD | ...LEDLKNVFPPEVAV | W3618-U4T1.E17R-1.uIgG1 comprises the heavy chains and |
| 1 | SKSSDKSVCLFTDFDSQT | FEPSEAEISHTQKATLV | light chains set forth in Table 23 as SEQ ID NO. 69, 70, |
| | QVSQSKDSDVYITDKCVL | CLATGFYPDHVELSWVN | 71 and 72, respectively. The amino acid residues of |
| | DMRSMDFKSNSAVAWSQK | GKEVHSGVCTDPQPLKE | C-terminal (92-95, numbering shown in FIG. 12) in Calpha |
| | SDFACANAFQNSIIPEDT | QPALQDSRYALSSRLRV | are bolded. The amino acid residues of a hinge region, |
| | FFPSPESS | SATFWQNPRNHFRCQVQ | attached to the C-terminus of the CBeta, are underlined. |
| | (SEQ ID NO: 10) | FYGLSENDEWTQDRAKP | As with the rest of the proteins in this chart, only the |
| | | VTQIVSAEAWGRASDKT | sequence around the CAlpha region and the sequence around |
| | | HTCPPCP... (SEQ | the C-Beta region of W3618-U4T1.E17R-1.uIgG1 are shown in |
| | | ID NO: 11) | this chart. |
| W3618- | ...PDIQNPDCAVYQLRD | ...LEDLKNVFPPEVAV | Introduce additional disulfide bond with P8C and D86C at |
| 57 | SKSSDKSVCLFTDFDSQT | FEPSEAEISHTQKATLV | Calpha (numbering shown in FIG. 12), delete 4 amino acids |
| | QVSQSKDSDVYITDKCVL | CLATGFYPDHVELSWVN | at TCR Calpha C terminal (92-95, numbering shown in FIG. |
| | DMRSMDFKSNSAVAWSQK | GKEVHSGVCTDPQPLKE | 12), with different amino acid residues bolded, in |
| | SDFACANAFQNSIIPECT | QPALQDSRYALSSRLRV | comparison to W3618-U4T1.E17R-1.uIgG1 |
| | EFPS (SEQ ID NO: | SATFWQNPRNHFRCQVQ | |
| | 42) | FYGLSENDEWTQDRAKP | |
| | | VTQIVSAEAWGRASDKT | |
| | | HTCPPCP... (SEQ | |
| | | ID NO: 43) | |
| W3618- | ...PDIQNPDPAVYQLRD | ...LEDLKNVFPPEVAV | Introduce additional disulfide bond with P90C at Calpha |
| 61 | SKSSDKSVCLFTDFDSQT | FEPSECEISHTQKATLV | (numbering shown in FIG. 12) and A18C at Cbeta (numbering |
| | QVSQSKDSDVYITDKCVL | CLATGFYPDHVELSWVN | shown in FIG. 13); delete 4 amino acids at TCR Calpha C |
| | DMRSMDEKSNSAVAWSQK | GKEVHSGVCTDPQPLKE | terminal (92-95, numbering shown in FIG. 12), with |
| | SDFACANAFQNSIIPEDT | QPALQDSRYALSSRLRV | different amino acid residues bolded, in comparison to |
| | FFCS (SEQ ID NO: | SATFWQNPRNHFRCQVQ | W3618-U4T1.E17R-1.uIgG1 |
| | 50) | FYGLSENDEWTQDRAKP | |
| | | VTQIVSAEAWGRASDKT | |
| | | HTCPPCP... (SEQ | |
| | | ID NO: 51) | |

### 2.2.2. Protein Production of W3618 WuXiBody™ Molecules

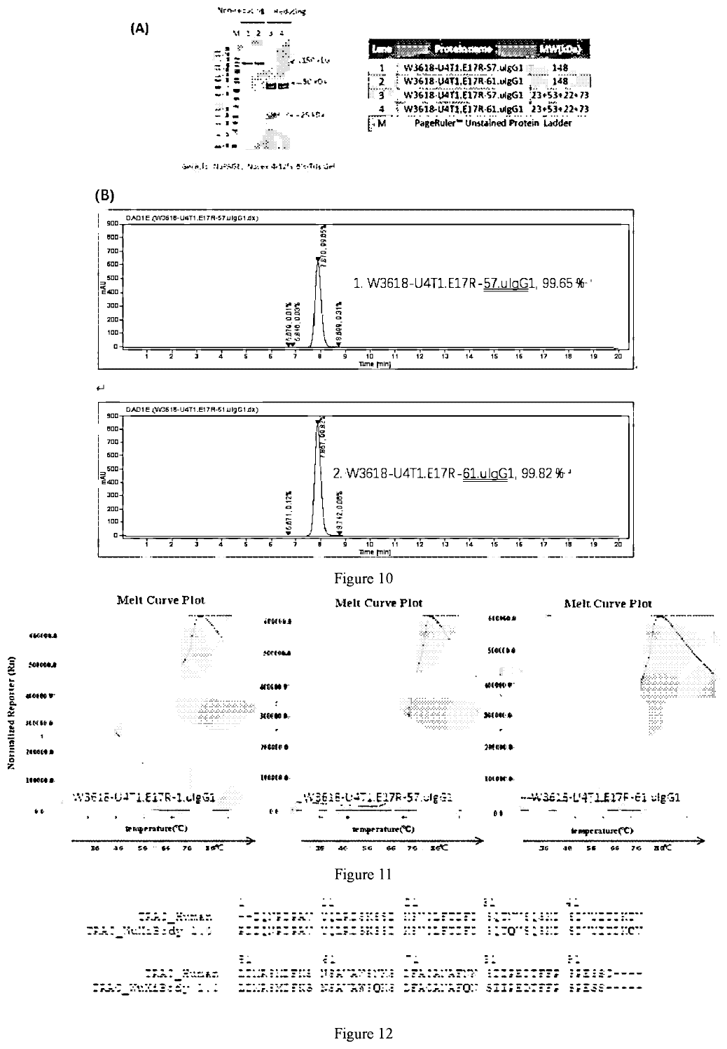

Table 11 summarizes the yield and purity of W3618-U4T1.E17R-1.uIgG1, W3618-U4T1.E17R-57.uIgG1, and W3618-U4T1.E17R-61.uIgG1 through transient expression in Expi293. After 3-step purification, the purities of all the bsAbs reached >90%. FIG. 10 shows the relevant SDS-PAGE and SEC-HPLC characterizations of the proteins after purification. These bsAbs migrated with the apparent molecular mass of 150 kDa under non-reducing condition, and 50 kDa, 25 kDa under reducing conditions, indicating the intact and well-assembled bispecific molecules.

TABLE 11
Summary of protein production of some designs of W3618 molecule.

| | | | First Batch | | |
|---|---|---|---|---|---|
| | | | Concen- | | Purity |
| | | | tration | Yield | by SEC- |
| Protein Name | PI | Buffer | (mg/ml) | (mg/L) | HPLC (%) |
| W3618-U4T1.E17R-1.uIgG1 | 6.6 | PBS | | | |
| W3618-U4T1.E17R-57.uIgG1 | 6.69 | | 2.31 | 30.02 | 99.65% |
| W3618-U4T1.E17R-61.uIgG1 | 6.6 | | 3.09 | 30.86 | 99.82% |

### 2.2.3. DSF of W3618 WuXiBody™ Molecules

The thermostability of the purified proteins of W3618 series were characterized by DSF, and the results were listed in Table 12. Compared to the reference WuXiBody™ molecule (W3618-U4T1.E17R-1.uIgG1), Design-57 (W3618-U4T1.E17R-57.uIgG1) and Design-61 (W3618-U4T1.E17R-61.uIgG1) improved Tm-onset by 2-4° C., and improved Tm1 by 4° C.

TABLE 12
Summary of DSF characterizations of W3618 molecules.

| | | | First Batch | | |
|---|---|---|---|---|---|
| | | | Concen- | | |
| | | | tration | Ton | Tm1 |
| Protein Name | pI | Buffer | (mg/ml) | (° C.) | (° C.) |
| W3618-U4T1.E17R-1.uIgG1 | 6.6 | PBS | 2.6 | 56 | 63.6 |
| W3618-U4T1.E17R-57.uIgG1 | 6.69 | | 2.3 | 60 | 67.2 |
| W3618-U4T1.E17R-61.uIgG1 | 6.6 | | 3.1 | 58 | 67.0 |

FIG. 11 shows the DSF profiles of W3618 WuXiBody™ molecules upon temperature increase.

### 2.2.4. Rodent PK

As shown in FIG. 15, Design-57 and Design-61 in W3618 had improved drug exposure significantly. Compared to the clearance of 9.94 ml/day/kg of the reference molecule (W3618-U4T1.E17R-1.uIgG1), the clearance of W3618-U4T1.E17R-57.uIgG1 and W3618-U4T1.E17R-61.uIgG1 was reduced to 6.35 ml/day/kg and 5.54 ml/day/kg, respectively. The half-life of W3618-U4T1.E17R-1.uIgG1 was also greatly improved by Design-57 and Design-61, from 176 hours to 289 hours and 388 hours, respectively (Table 13). This was a more than 40% improvement, and achieved the level close to a regular monoclonal antibody.

TABLE 13
PK results of W3618 WuXiBody ™ molecules

| | G6_W3618- | G7_W3618- | G8_W3618- |
|---|---|---|---|
| | U4T1.E17R- | U4T1.E17R- | U4T1.E17R- |
| Compound | 1.uIgG1 | 57.uIgG1 | 61.uIgG1 |
| Dose(mg/kg) | 10mg/kg, i.v. | | |
| t_{1/2} (h) | 176 | 289 | 388 |
| Cₘₐₓ (μg/mL) | 199 | 185 | 193 |
| AUC₀₋ₜ (h*μg/ml) | 20700 | 26543 | 23992 |
| Cl_obs (ml/day/kg) | 9.94 | 6.35 | 5.54 |
| MRTINF_obs (h) | 258 | 413 | 554 |
| Vss_obs (mL/kg) | 106 | 109 | 128 |

### 2.3.1. WuXiBody™ Engineering on W329001-U3T3 WuXiBody™ Molecules (2+2, Symmetric)

TABLE 14
Design details in W329001-U3T3 symmetric 2+2 format WuXiBody ™ molecule

| W329001- | | | |
|---|---|---|---|
| U3T3. | | | |
| G25R-?. | | | |
| uIgG1 | TCR alpha sequence | TCR beta sequence | Comments |
| W329001- | ...PDIQNPDPAVYQLRD | ...LEDLKNVEPPEVAV | W329001-U3T3.G25R-1.uIgG1 comprises the heavy chains |
| U3T3-1 | SKSSDKSVCLFTDFDSQT | FEPSEAEISHTQKATLV | and light chains set forth in Table 24 as SEQ ID NO. |
| | QVSQSKDSDVYITDKCVL | CLATGFYPDHVELSWVN | 73, 74 and 75, respectively. The amino acid residues |
| | DMRSMDFKSNSAVAWSQK | GKEVHSGVCTDPQPLKE | of C-terminal (92-95, numbering shown in FIG. 12) in |
| | SDFACANAFQNSIIPEDT | QPALQDSRYALSSRLRV | Calpha are bolded. The amino acid residues of a hinge |
| | FFPSPESS (SEQ ID | SATFWQNPRNHFRCQVQ | region, attached to the C-terminus of the CBeta, are |
| | NO: 10) | FYGLSENDEWTQDRAKP | underlined. As with the rest of the proteins in this |
| | | VTQIVSAEAWGRASDKT | chart, only the sequence around the CAlpha region and |
| | | HTCPPCP... (SEQ | the sequence around the C-Beta region of W329001- |
| | | ID NO: 11) | U3T3.G25R-1.uIgG1 are shown in this chart. |
| W329001- | ...PDIQNPDCAVYQLRD | ...LEDLKNVFPPEVAV | Introduce additional disulfide bond with P8C and D86C |
| U3T3-57 | SKSSDKSVCLFTDFDSQT | FEPSEAEISHTQKATLV | at Calpha (numbering shown in FIG. 12), delete 4 amino |
| | QVSQSKDSDVYITDKCVL | CLATGFYPDHVELSWVN | acids at TCR Calpha C terminal (92-95, numbering shown |
| | DMRSMDFKSNSAVAWSQK | GKEVHSGVCTDPQPLKE | in FIG. 12), with different amino acid residues bolded, |
| | SDFACANAFQNSIIPECT | QPALQDSRYALSSRLRV | in comparison to W329001-U3T3.G25R-1.uIgG1 |
| | EFPS (SEQ ID NO: | SATFWQNPRNHFRCQVQ | |
| | 42) | FYGLSENDEWTQDRAKP | |
| | | VTQIVSAEAWGRASDKT | |
| | | HTCPPCP... (SEQ | |
| | | ID NO: 43) | |
| W329001- | ...PDIQNPDPAVYQLRD | ...LEDLKNVFPPEVAV | Introduce additional disulfide bond with P90C at Calpha |
| U3T3-61 | SKSSDKSVCLFTDFDSQT | FEPSECEISHTQKATLV | (numbering shown in FIG. 12) and A18C at Cbeta |
| | QVSQSKDSDVYITDKCVL | CLATGFYPDHVELSWVN | (numbering shown in FIG. 13); delete 4 amino acids at |
| | DMRSMDFKSNSAVAWSQK | GKEVHSGVCTDPQPLKE | TCR Calpha C terminal (92-95, numbering shown in FIG. |
| | SDFACANAFQNSIIPEDT | QPALQDSRYALSSRLRV | 12), with different amino acid residues bolded, in |
| | FFCS (SEQ ID NO: | SATFWQNPRNHFRCQVQ | comparison to W329001-U3T3.G25R-1.uIgG1 |
| | 50) | FYGLSENDEWTQDRAKP | |
| | | VTQIVSAEAWGRASDKT | |
| | | HTCPPCP... (SEQ | |
| | | ID NO: 51) | |

### 2.3.2. Protein Production of W329001-U3T3 WuXiBody™ Molecules

The W329001-U3T3 WuXiBody™ molecules were generated based on the publicly available sequences of FIT-Ig (1L17×IL20) (see WO 2017/136820, FIT-Ig) (named W329001-U3T3.G25R-1.uIgG1).

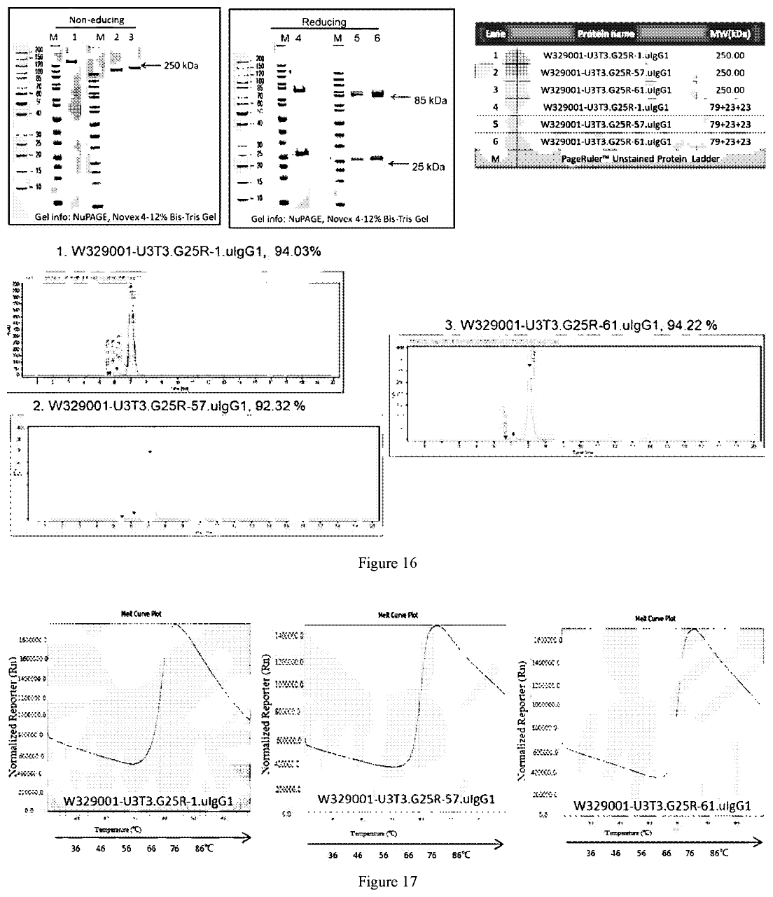

Table 15 summarizes the yield and purity of W329001-U3T3.G25R-1.uIgG1, W329001-U3T3.G25R-57.uIgG1, and W329001-U3T3.G25R-61.uIgG1 through transient expression in Expi293. After a 2-step purification, the purities of all of these bsAbs reached >90%. FIG. 16 shows the relevant SDS-PAGE and SEC-HPLC characterizations of the proteins after purification. These bsAbs migrated with the apparent molecular mass of 250 kDa under non-reducing condition, and 75 kDa, 25 kDa and 25 kDa under reducing conditions, indicating the intact and well-assembled bispecific molecules.

TABLE 15
Summary of protein production of some designs of W329001-U3T3 molecules.

| | | | First Batch | | |
|---|---|---|---|---|---|
| | | | Concen- | | Purity |
| | | | tration | Yield | by SEC- |
| Protein Name | PI | Buffer | (mg/ml) | (mg/L) | HPLC (%) |
| W329001-U3T3.G25R-1.uIgG1 | 6.4 | PBS | | | |
| W329001-U3T3.G25R-57.uIgG1 | 6.6 | | 2.6 | 28.28 | 92.32% |
| W329001-U3T3.G25R-61.uIgG1 | 6.5 | | 2.7 | 32.14 | 94.22% |

### 2.3.3. DSF of W329001-U3T3 WuXiBody™ Molecules

The thermostability of the purified proteins of W329001-U3T3 series were characterized by DSF, and the results were listed in Table 16. Compared to the reference WuXiBody™ molecule (W329001-U3T3.G25R-1.uIgG1), Design-57 (W329001-U3T3.G25R-57.uIgG1) and Design-61 (W329001-U3T3.G25R-61.uIgG1) improved Tm1 by 1° C.

TABLE 16
Summary of DSF characterizations of W329001-U3T3 molecules.

| | | | First Batch | |
|---|---|---|---|---|
| | | | Concentration | Tm1 |
| Protein Name | pI | Buffer | (mg/ml) | (° C.) |
| W329001-U3T3.G25R-1.uIgG1 | 6.4 | PBS | 3.1 | 64.6 |
| W329001-U3T3.G25R-57.uIgG1 | 6.6 | | 2.6 | 65.2 |
| W329001-U3T3.G25R-61.uIgG1 | 6.5 | | 2.7 | 65.2 |

FIG. 17 shows the DSF profiles of W329001-U3T3 WuXiBody™ molecules upon temperature increase.

### 2.3.4. Rodent PK

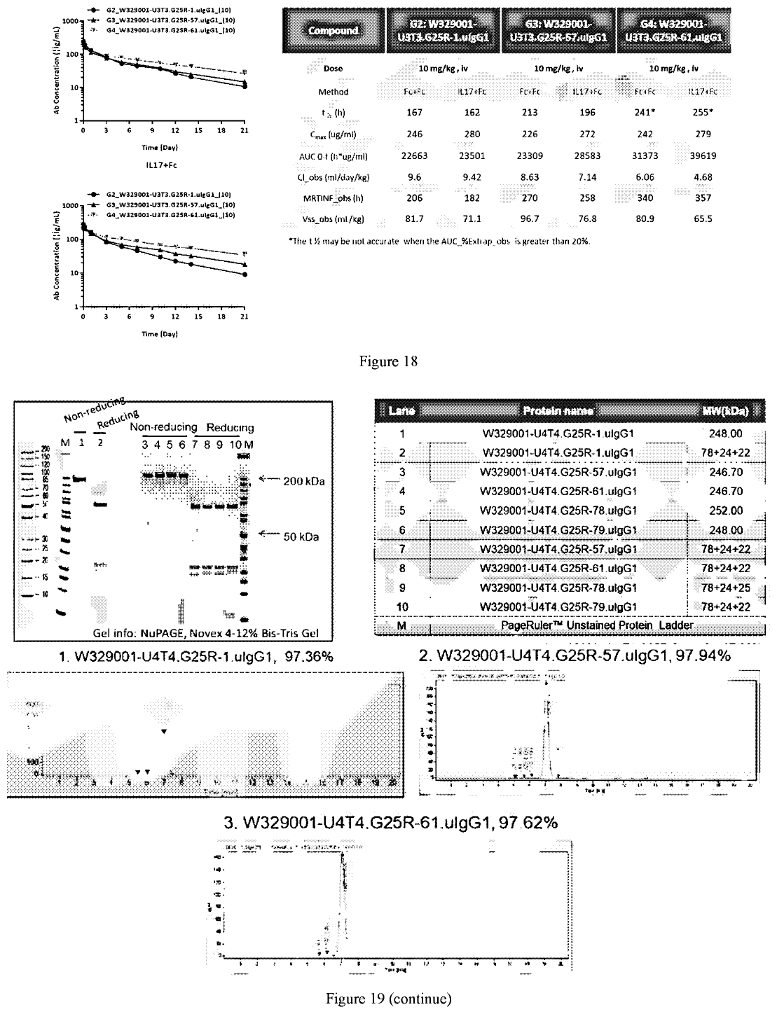

As shown in FIG. 18, Design-57 and Design-61 in W329001-U3T3 had improved drug exposure significantly. Compared to the clearance of 9.6 ml/day/kg of the reference molecule (W329001-U3T3.G25R-1.uIgG1), the clearance of W329001-U3T3.G25R-57.uIgG1 and W329001-U3T3.G25R-61.uIgG1 was reduced to 8.63 ml/day/kg and 6.06 ml/day/kg, respectively. The half-life was also improved by Design-57 and Design-61, compared to W329001-U3T3.G25R-1.uIgG1, from 167 hours to 213 hours and 341 hours, respectively (Table 17). Similar improvement was also observed when being detected by an antigen-Fc ELISA method. It was a great improvement, and achieved the level close to a regular monoclonal antibody.

TABLE 17
PK results of W329001-U3T3 WuXiBody ™ molecules

| | Compound | | | | | |
|---|---|---|---|---|---|---|
| | G2: W329001- | | G3: W329001- | | G4: W329001- | |
| | U3T3.G25R-1.uIgG1 | | U3T3.G25R-57.uIgG1 | | U3T3.G25R-61.uIgG1 | |
| | Dose | | | | | |
| | 10 mg/kg, iv | | | | | |
| | Method | | | | | |
| | Fc + Fc | IL17 + Fc | Fc + Fc | IL17 + Fc | Fc + Fc | IL17 + Fc |
| t ½ (h) | 167 | 162 | 213 | 196 | 241* | 255* |
| Cmax (ug/ml) | 246 | 280 | 226 | 272 | 242 | 279 |
| AUC 0-t (h*ug/ml) | 22663 | 23501 | 23309 | 28583 | 31373 | 39619 |
| Cl_obs (ml/day/kg) | 9.6 | 9.42 | 8.63 | 7.14 | 6.06 | 4.68 |
| MRTINF_obs (h) | 206 | 182 | 270 | 258 | 340 | 357 |
| Vss_obs (mL/kg) | 81.7 | 71.1 | 96.7 | 76.8 | 80.9 | 65.5 |

### 2.4.1. WuXiBody™ Engineering on W329001-U4T4 WuXiBody™ Molecules (2+2, Symmetric)

TABLE 18
Design details in W329001-U4T4 symmetric 2 + 2 format WuXiBody ™ molecule

| W329001- | | | |
|---|---|---|---|
| U4T4. | | | |
| G25R-?. | | | |
| uIgG1 | TCR alpha sequence | TCR beta sequence | Comments |
| W329001- | ...PDIQNPDPAVYQLRD | ...LEDLKNVFPPEVAV | W329001-U4T4.G25R-1.uIgG1 comprises the heavy chains |
| U4T4-1 | SKSSDKSVCLFTDFDSQT | FEPSEAEISHTQKATLV | and light chains set forth in Table 25 as SEQ ID NO. |
| | QVSQSKDSDVYITDKCVL | CLATGFYPDHVELSWVN | 76, 77 and 78, respectively. The amino acid residues |
| | DMRSMDFKSNSAVAWSQK | GKEVHSGVCTDPQPLKE | of C-terminal (92-95, numbering shown in FIG. 12) in |
| | SDFACANAFQNSIIPEDT | QPALQDSRYALSSRLRV | Calpha are bolded. The amino acid residues of a hinge |
| | FFPSPESS (SEQ ID | SATFWQNPRNHFRCQVQ | region, attached to the C-terminus of the CBeta, are |
| | NO: 10) | FYGLSENDEWTQDRAKP | underlined. As with the rest of the proteins in this |
| | | VTQIVSAEAWGRASDKT | chart, only the sequence around the CAlpha region and |
| | | HTCPPCP... (SEQ | the sequence around the C-Beta region of W329001-U4T4. |
| | | ID NO: 11) | G25R-1.uIgG1 are shown in this chart. |
| W329001- | ...PDIQNPDCAVYQLRD | ...LEDLKNVFPPEVAV | Introduce additional disulfide bond with P8C and D86C |
| U4T4-57 | SKSSDKSVCLFTDFDSQT | FEPSEAEISHTQKATLV | at Calpha (numbering shown in FIG. 12), delete 4 amino |
| | QVSQSKDSDVYITDKCVL | CLATGFYPDHVELSWVN | acids at TCR Calpha C terminal (92-95, numbering shown |
| | DMRSMDFKSNSAVAWSQK | GKEVHSGVCTDPQPLKE | in FIG. 12), with different amino acid residues bolded, |
| | SDFACANAFQNSIIPECT | QPALQDSRYALSSRLRV | in comparison to W329001-U4T4.G25R-1.uIgG1 |
| | FFPS (SEQ ID NO: | SATFWQNPRNHFRCQVQ | |
| | 42) | FYGLSENDEWTQDRAKP | |
| | | VTQIVSAEAWGRASDKT | |
| | | HTCPPCP... (SEQ | |
| | | ID NO: 43) | |
| W329001- | ...PDIQNPDPAVYQLRD | ...LEDLKNVEPPEVAV | Introduce additional disulfide bond with P90C at Calpha |
| U4T4-61 | SKSSDKSVCLFTDFDSQT | FEPSECEISHTQKATLV | (numbering shown in FIG. 12) and A18C at Cbeta |
| | QVSQSKDSDVYITDKCVL | CLATGFYPDHVELSWVN | (numbering shown in FIG. 13); delete 4 amino acids at |
| | DMRSMDFKSNSAVAWSQK | GKEVHSGVCTDPQPLKE | TCR Calpha C terminal (92-95, numbering shown in FIG. |
| | SDFACANAFQNSIIPEDT | QPALQDSRYALSSRLRV | 12), with different amino acid residues bolded, in |
| | FFCS (SEQ ID NO: | SATFWQNPRNHFRCQVQ | comparison to W329001-U4T4.G25R-1.uIgG1 |
| | 50) | FYGLSENDEWTQDRAKP | |
| | | VTQIVSAEAWGRASDKT | |
| | | HTCPPCP... (SEQ | |
| | | ID NO: 51) | |
| W329001- | ...PDIQNPDCAVYQLRD | ...LEDLKNVFPPEVAV | Introduce additional disulfide bond with P8C and D86C |
| U4T4-78 | SKSSDKSVCLFTDFDSQT | FEPSEAEISHTQKATLV | at Calpha (numbering shown in FIG. 12), with different |
| | QVSQSKDSDVYITDKCVL | CLATGFYPDHVELSWVN | amino acid residues bolded, in comparison to W329001- |
| | DMRSMDFKSNSAVAWSQK | GKEVHSGVCTDPQPLKE | U4T4.G25R-1.uIgG1 |
| | SDFACANAFQNSIIPECT | QPALQDSRYALSSRLRV | |
| | FFPSPESS (SEQ ID | SATFWQNPRNHFRCQVQ | |
| | NO: 79) | FYGLSENDEWTQDRAKP | |
| | | VTQIVSAEAWGRASDKT | |
| | | HTCPPCP... (SEQ | |
| | | ID NO: 43) | |
| W329001- | ...PDIQNPDPAVYQLRD | ...LEDLKNVFPPEVAV | Introduce additional disulfide bond with P90C at Calpha |
| U4T4-79 | SKSSDKSVCLFTDFDSQT | FEPSECEISHTQKATLV | (numbering shown in FIG. 12) and A18C at Cbeta |
| | QVSQSKDSDVYITDKCVL | CLATGFYPDHVELSWVN | (numbering shown in FIG. 13); with different amino acid |
| | DMRSMDFKSNSAVAWSQK | GKEVHSGVCTDPQPLKE | residues bolded, in comparison to W329001-U4T4.G25R-1. |
| | SDFACANAFQNSIIPEDT | QPALQDSRYALSSRLRV | uIgG1 |
| | FFCSPESS (SEQ ID | SATFWQNPRNHFRCQVQ | |
| | NO: 80) | FYGLSENDEWTQDRAKP | |
| | | VTQIVSAEAWGRASDKT | |
| | | HTCPPCP... (SEQ | |
| | | ID NO: 51) | |

### 2.4.2. Protein Production of W329001-U4T4 WuXiBody™ Molecules

The W329001-U4T4 WuXiBody™ molecules were generated based on the publicly available sequences of CODV-Ig (ILA×IL13) (see Anke Steinmetz et al., “CODV-Ig, a universal bispecific tetravalent and multifunctional immunoglobulin format for medical applications”, MARS, 2016, VOL. 8, NO. 5, 867-878, CODV-Ig).

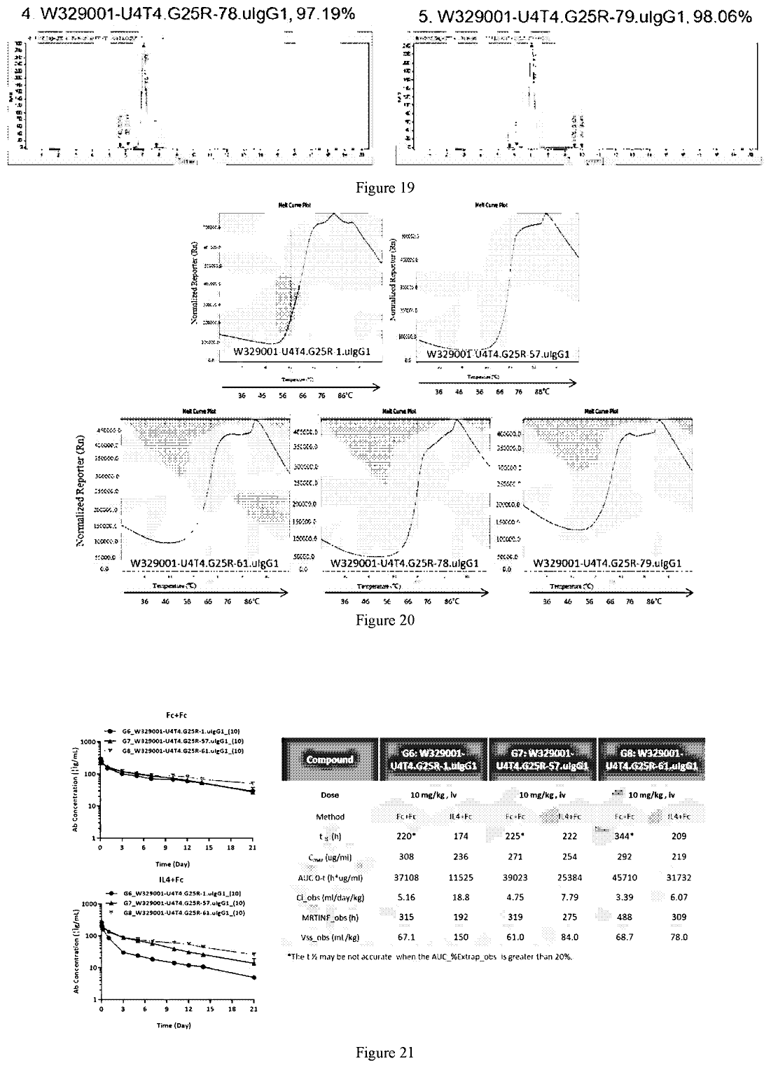

Table 19 summarizes the yield and purity of proteins of W329001-U4T4.G25R-1.uIgG1, W329001-U4T4.G25R-57.uIgG1, W329001-U4T4.G25R-61.uIgG1, W329001-U4T4.G25R-78.uIgG1, and W329001-U4T4.G25R-79.uIgG1 through transient expression in Expi293. After a 1-step purification, the purifies of all of these bsAbs reached >90%. FIG. 19 shows the relevant SDS-PAGE and SEC-HPLC characterizations of the proteins after purification. These bsAbs migrated with the apparent molecular mass of 250 kDa under non-reducing condition, and 75 kDa, 25 kDa and 25 kDa under reducing conditions, indicating the intact and well-assembled bispecific molecules.

TABLE 19
Summary of protein production of some designs of W329001-U4T4 molecule.

| | | | First Batch | | |
|---|---|---|---|---|---|
| | | | Concen- | | Purity |
| | | | tration | Yield | by SEC- |
| Protein Name | PI | Buffer | (mg/ml) | (mg/L) | HPLC (%) |
| W329001-U4T4.G25R-1.uIgG1 | 5.8 | PBS | | | |
| W329001- U4T4.G25R-57.uIgG1 | 5.8 | | 5.7 | 22.92 | 97.47% |
| W329001- U4T4.G25R-61.uIgG1 | 5.8 | | 4.1 | 46.32 | 97.70% |
| W329001- U4T4.G25R-78.uIgG1 | 5.8 | | 4.4 | 32.08 | 97.19% |
| W329001- U4T4.G25R-79.uIgG1 | 5.8 | | 3.2 | 31.97 | 98.06% |

### 2.4.3. DSF of W329001-U4T4 WuXiBody™ Molecules

The thermostability of purified proteins of the W329001-U4T4 series was characterized by DSF, and the results are listed in Table 20. Compared to the reference WuXiBody™ molecule (W329001-U4T4.G25R-1.uIgG1), Design-57 (W329001-U4T4.G25R-57.uIgG1), Design-61 (W329001-U4T4.G25R-61.uIgG1), Design-78 (W329001-U4T4.G25R-78.uIgG1), and Design-79 (W329001-U4T4.G25R-79.uIgG1) improved Tm1 by 6-9° C.

TABLE 20
Summary of DSF characterizations of W329001-U4T4 molecules.

| | | | First Batch | |
|---|---|---|---|---|
| | | | Concentration | Tm1 |
| Protein Name | pI | Buffer | (mg/ml) | (° C.) |
| W329001-U4T4.G25R-1.uIgG1 | 5.8 | PBS | 1.6 | 56.4 |
| W329001- U4T4.G25R-57.uIgG1 | 5.8 | | 2.9 | 66.0 |
| W329001- U4T4.G25R-61.uIgG1 | 5.8 | | 2.5 | 62.8 |
| W329001- U4T4.G25R-78.uIgG1 | 5.8 | | 2.5 | 65.6 |
| W329001- U4T4.G25R-79.uIgG1 | 5.8 | | 2.6 | 62.4 |

FIG. 20 shows the DSF profiles of W329001-U4T4 WuXiBody™ molecules upon temperature increase.

### 2.4.4. Rodent PK

As shown in FIG. 21, Design-57 and Design-61 in W329001-U4T4 had improved drug exposure significantly. Compared to the clearance of 5.16 ml/day/kg of the reference molecule (W329001-U4T4.G25R-1.uIgG1), the clearance of W329001-U4T4.G25R-57.uIgG1 and W329001-U4T4.G25R-61.uIgG1 was reduced to 4.75 ml/day/kg and 3.39 ml/day/kg, respectively. The half-life was also improved by Design-57 and Design-61, compared to W329001-U4T4.G25R-1.uIgG1, from 220 hours to 225 hours and 344 hours, respectively (Table 21). Similar improvement was also observed when being detected by an antigen-Fc ELISA method. It was a great improvement, and achieved the level close to a regular monoclonal antibody.

In FIG. 22 and FIG. 23, when comparing the rat PK results between Design-57 and Design-78 (Design-57 with Calpha C-terminal PESS added) as well as Design-61 and Design-79 (Design-61 with Calpha C-terminal PESS added), no significant difference was observed by either Fc-Fc or antigen-Fc detection method (Table 21). Characterizations by DSF (FIG. 20) and O-glycosylation (data not shown) did not indicate obvious differences, either.

TABLE 21A
PK results of W329001-U4T4 WuXiBody ™ molecules

| | Compound | | | | | |
|---|---|---|---|---|---|---|
| | G6: W329001- | | G7: W329001- | | G8: W329001- | |
| | U4T4.G25R-1.uIgG1 | | U4T4.G25R-57.uIgG1 | | U4T4.G25R-61.uIgG1 | |
| | Dose | | | | | |
| | 10 mg/kg, iv | | | | | |
| | Method | | | | | |
| | Fc + Fc | IL4 + Fc | Fc + Fc | IL4 + Fc | Fc + Fc | IL4 + Fc |
| t ½ (h) | 220* | 174 | 225* | 222 | 344* | 209 |
| Cmax (ug/ml) | 308 | 236 | 271 | 254 | 292 | 219 |
| AUC 0-t (h*ug/ml) | 37108 | 11525 | 39023 | 25384 | 45710 | 31732 |
| Cl_obs (ml/day/kg) | 5.16 | 18.8 | 4.75 | 7.79 | 3.39 | 6.07 |
| MRTINF_obs (h) | 315 | 192 | 319 | 275 | 488 | 309 |
| Vss_obs (mL/kg) | 67.1 | 150 | 61 | 84 | 68.7 | 78 |

TABLE 21B
PK results of W329001-U4T4 WuXiBody ™ molecules

| | Compound | | | | | | | |
|---|---|---|---|---|---|---|---|---|
| | W329001- | | W329001- | | W329001- | | W329001- | |
| | U4T4.G25R-57.uIgG1 | | U4T4.G25R-78.uIgG1 | | U4T4.G25R-61.uIgG1 | | U4T4.G25R-79.uIgG1 | |
| | Method | | | | | | | |
| | Fc + Fc | IL4 + Fc | Fc + Fc | IL4 + Fc | Fc + Fc | IL4 + Fc | Fc + Fc | IL4 + Fc |
| | Dose (mg/kg , iv) | | | | | | | |
| | 10 | | | | | | | |
| t ½ (h) | 220\hat{ } | 162\hat{ } | 203 | 181 | 203 | 143 | 305\hat{ } | 199*\hat{ } |
| Cmax (ug/ml) | 196 | 135 | 195 | 153 | 168 | 100 | 195 | 178 |
| AUC 0-t (h*ug/ml) | 22612 | 13820 | 26003 | 20268 | 21372 | 15657 | 23570 | 14850 |
| Cl_obs (ml/day/kg) | 7.93 | 15.8 | 7.59 | 10 | 9.26 | 13.9 | 6.84 | 13.1 |
| MRTINF_obs (h) | 312 | 213 | 290 | 260 | 292 | 217 | 418 | 271 |
| Vss_obs (mL/kg) | 99.5 | 130 | 90.3 | 108 | 112 | 126 | 115 | 142 |

TABLE 22
Full length sequences of T8311-U14T2.G25R-1.uIgG1

| T8311- | 23 pYT8311- | QVQLQESGPGLVKPSETLSLTCTVSGFSLTENSVSWIRQPPGKGLEWIGAVWSSGS |
|---|---|---|
| U14T2. | U14T2.G25R- | TDYNSALKSRVTISRDTSKNQFSLKLSSVTAADTAVYYCTRSTYSNDFYYYFDYWG |
| G25R-1. | 1.uIgG1(dk) | QGTMVTVSSASTKGPSVFPLAPSSKSTSGGTAALGCLVKDYFPEPVTVSWNSGALT |
| uIgG1 | (SEQ ID NO: | SGVHTFPAVLQSSGLYSLSSVVTVPSSSLGTQTYICNVNHKPSNTKVDKRVEPKSC |
| | 66) | GGGGSGGGGSQEQLQESGPGLVKPSQTLSLTCTVSGGSINSQGYYWSWIRQHPGKG |
| | | LEWIGYIYDSGSAYYNPSLERRVAISLDTSKNQFSLNLNSVTVADTAVYYCARIVA |
| | | AGRIDPWGQGTLVTVLEDLKNVFPPEVAVFEPSEAEISHTQKATLVCLATGFYPDH |
| | | VELSWWVNGKEVHSGVCTDPQPLKEQPALQDSRYALSSRLRVSATFWQNPRNHFRC |
| | | QVQFYGLSENDEWTQDRAKPVTQIVSAEAWGRASDKTHTCPPCPAPELLGGPSVEL |
| | | FPPKPKDTLMISRTPEVTCVVVDVSHEDPEVKENWYVDGVEVHNAKTKPREEQYNS |
| | | TYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFFLYSKL |
| | | TVDKSRWQQGNVFSCSVMHEALHNHYTQKSLSLSPG |
| | 1 pYW3668- | SYELTQPPSVSVSPGQTASITCSGSELPKRYAYWYQQKPGQSIVRVIYKDSERPSG |
| | U14.L | ISERFSGSSSGNTATLTISGTQAMDEADYYCSSTYGDRKLPIFGGGTKLTVLGQPK |
| | (SEQ ID NO: | AAPSVTLFPPSSEELQANKATLVCLISDFYPGAVTVAWKADSSPVKAGVETTTPSK |
| | 67) | QSNNKYAASSYLSLTPEQWKSHKSYSCQVTHEGSTVEKTVAPTECS |
| | 29 pYW-XJ- | SYVLTQPPSVSVAPGQTARMTCGGDNIGIKIVHWYQQKAGQAPVLVVYDDNDRPSG |
| | T2-hLA | IPDRFSGSNSGNTATLTISRVAAGDEADYYCQVWDRRSDHVVFGGGTKLTVLPDIQ |
| | (SEQ ID NO: | NPDPAVYQLRDSKSSDKSVCLFTDFDSQTQVSQSKDSDVYITDKCVLDMRSMDFKS |
| | 68) | NSAVAWSQKSDFACANAFQNSIIPEDTFFPSPESS |

TABLE 23
Full length sequences of W3618-U4T1.E17R-1.uIgG1

| | | |
|---|---|---|
| W3618- | 10 pYW3618- | EVQLVESGGGLVQPGGSLRLSCAASGFTFTDYTMDWVRQAPGKGLEWVADVNPNSG |
| U4T1. | U4-1.uIgG1. | GSIYNQRFKGRFTLSVDRSKNTLYLQMNSLRAEDTAVYYCARNLGPSFYFDYWGQG |
| E17R-1. | k(dk) (SEQ | TLVTVSSASTKGPSVFPLAPSSKSTSGGTAALGCLVKDYFPEPVTVSWNSGALTSG |
| uIgG1 | ID NO: 69) | VHTFPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNKALPAPIEK |
| | | TISKAKGQPREPQVYTLPPCREEMTKNQVSLWCLVKGFYPSDIAVEWESNGQPENN |
| | | YKTTPPVLDSDGSFFLYSKLTVDKSRWQQGNVFSCSVMHEALHNHYTQKSLSLSPG |
| | 5 pYW3618- | EVQLVESGGGLVQPGGSLRLSCAASGFNIKDTYIHWVRQAPGKGLEWVARIYPTNG |
| | T1-TBeta(2) | YTRYADSVKGRFTISADTSKNTAYLQMNSLRAEDTAVYYCSRWGGDGFYAMDYWGQ |
| | (Q)-1.uIgG1. | GTLVTVLEDLKNVFPPEVAVFEPSEAEISHTQKATLVCLATGFYPDHVELSWWVNG |
| | h(dk) (SEQ | KEVHSGVCTDPQPLKEQPALQDSRYALSSRLRVSATFWQNPRNHFRCQVQFYGLSE |
| | ID NO: 70) | NDEWTQDRAKPVTQIVSAEAWGRASDKTHTCPPCPAPELLGGPSVFLFPPKPKDTL |
| | | MISRTPEVTCVVVDVSHEDPEVKENWYVDGVEVHNAKTKPREEQYNSTYRVVSVLT |
| | | VLHQDWLNGKEYKCKVSNKALPAPIEKTISKAKGQPREPQVCTLPPSREEMTKNQV |
| | | SLSCAVKGFYPSDIAVEWESNGQPENNYKTTPPVLDSDGSFFLVSKLTVDKSRWQQ |
| | | GNVFSCSVMHEALHNHYTQKSLSLSPG |
| | 13 pYW361- | DIQMTQSPSSLSASVGDRVTITCKASQDVSIGVAWYQQKPGKAPKLLIYSASYRYT |
| | U4-hCk (SEQ | GVPSRFSGSGSGTDFTLTISSLQPEDFATYYCQQYYIYPYTFGQGTKVEIKRTVAA |
| | ID NO: 71) | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSENRGEC |
| | 1 pYW3618- | DIQMTQSPSSLSASVGDRVTITCRASQDVNTAVAWYQQKPGKAPKLLIYSASFLYS |
| | T1-hCA (SEQ | GVPSRFSGSRSGTDFTLTISSLQPEDFATYYCQQHYTTPPTFGQGTKVEIKPDIQN |
| | ID NO: 72) | PDPAVYQLRDSKSSDKSVCLFTDFDSQTQVSQSKDSDVYITDKCVLDMRSMDFKSN |
| | | SAVAWSQKSDFACANAFQNSIIPEDTFFPSPESS |

TABLE 24
Full length sequences of W329001-U3T3.G25R-1.uIgG1

| | | |
|---|---|---|
| W329001- | 15 | QVQLVQSGAEVKRPGASVKVSCKASGYTFTNDIIHWVRQAPGQRLEWMGWINAGYG |
| U3T3. | pYW329001- | NTQYSQNFQDRVSITRDTSASTAYMELISLRSEDTAVYYCAREPLWFGESSPHDYY |
| G25R-1. | U3T3.G25R- | GMDWGQGTTVTVSSASTKGPSVFPLAPSSKSTSGGTAALGCLVKDYFPEPVTVSWN |
| uIgG1 | 1.uIgG1(dk) | SGALTSGVHTFPAVLQSSGLYSLSSVVTVPSSSLGTQTYICNVNHKPSNTKVDKRV |
| | (SEQ ID NO: | EPKSCGGGGSGGGGSQVQLVQSGAEVKKPGSSVKVSCKASGYSFTDYHIHWVRQAP |
| | 73) | GQGLEWMGVINPMYGTTDYNQRFKGRVTITADESTSTAYMELSSLRSEDTAVYYCA |
| | | RYDYFTGTGVYWGQGTLVTVLEDLKNVFPPEVAVFEPSEAEISHTQKATLVCLATG |
| | | FYPDHVELSWWVNGKEVHSGVCTDPQPLKEQPALQDSRYALSSRLRVSATFWQNPR |
| | | NHFRCQVQFYGLSENDEWTQDRAKPVTQIVSAEAWGRASDKTHTCPPCPAPELLGG |
| | | PSVFLFPPKPKDTLMISRTPEVTCVVVDVSHEDPEVKENWYVDGVEVHNAKTKPRE |
| | | EQYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| | 19 | AIQLTQSPSSLSASVGDRVTITCRASQGISSALAWYQQKPGKAPKLLIYDASSLES |
| | pYW329001- | GVPSRFSGSGSGTDFTLTISSLQPEDFATYYCQQFNSYPLTFGGGTKVEIKRTVAA |
| | U3.hCk | PSVFIFPPSDEQLKSGTASVVCLLNNFYPREAKVQWKVDNALQSGNSQESVTEQDS |
| | (SEQ ID NO: | KDSTYSLSSTLTLSKADYEKHKVYACEVTHQGLSSPVTKSENRGEC |
| | 74) | |
| | 23 | DIVMTQTPLSLSVTPGQPASISCRSSRSLVHSRGNTYLHWYLQKPGQSPQLLIYKV |
| | pYW329001- | SNRFIGVPDRFSGSGSGTDFTLKISRVEAEDVGVYYCSQSTHLPFTFGQGTKLEIK |
| | T3.hCA | PDIQNPDPAVYQLRDSKSSDKSVCLFTDFDSQTQVSQSKDSDVYITDKCVLDMRSM |
| | (SEQ ID NO: | DFKSNSAVAWSQKSDFACANAFQNSIIPEDTFFPSPESS |
| | 75) | |

TABLE 25
Full length sequences of W329001-U4T4.G25R-1.uIgG1

| | | |
|---|---|---|
| W329001- | 16 | EVQLKESGPGLVAPGGSLSITCTVSGFSLTDSSINWVRQPPGKGLEWLGMIWGDGR |
| U4T4. | pYW329001- | IDYADALKSRLSISKDSSKSQVFLEMTSLRTDDTATYYCARDGYFPYAMDFWGQGT |
| G25R-1. | U4T4.G25R- | SVTVSSASTKGPSVFPLAPSSKSTSGGTAALGCLVKDYFPEPVTVSWNSGALTSGV |
| uIgG1 | 1.uIgG1(dk) | HTFPAVLQSSGLYSLSSVVTVPSSSLGTQTYICNVNHKPSNTKVDKRVEPKSCGGG |
| | (SEQ ID NO: | GSGGGGSAVQLQQSGPELVKPGASVKISCKASGYSFTSYWIHWIKQRPGQGLEWIG |
| | 76) | MIDPSDGETRLNQRFQGRATLTVDESTSTAYMQLRSPTSEDSAVYYCTRLKEYGNY |
| | | DSFYFDVWGAGTLVTVLEDLKNVFPPEVAVFEPSEAEISHTQKATLVCLATGFYPD |
| | | HVELSWWVNGKEVHSGVCTDPQPLKEQPALQDSRYALSSRLRVSATFWQNPRNHFR |
| | | CQVQFYGLSENDEWTQDRAKPVTQIVSAEAWGRASDKTHTCPPCPAPELLGGPSVE |
| | | LFPPKPKDTLMISRTPEVTCVVVDVSHEDPEVKENWYVDGVEVHNAKTKPREEQYN |
| | | STYRVVSVLTVLHQDWLNGKEYKCKVSNKALPAPIEKTISKAKGQPREPQVYTLPP |
| | | SREEMTKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| | 20 | DIVLTQSPASLAVSLGQRATISCRASESVDSYGQSYMHWYQQKAGQPPKLLIYLAS |
| | pYW329001- | NLESGVPARFSGSGSRTDFTLTIDPVQAEDAATYYCQQNAEDSRTFGGGTKLEIKR |
| | U4.hCk | TVAAPSVFIFPPSDEQLKSGTASVVCLLNNFYPREAKVQWKVDNALQSGNSQESVT |
| | (SEQ ID NO: | EQDSKDSTYSLSSTLTLSKADYEKHKVYACEVTHQGLSSPVTKSENRGEC |
| | 77) | |
| | 24 | DIQMTQSPASLSVSVGDTITLTCHASQNIDVWLSWFQQKPGNIPKLLIYKASNLHT |
| | pYW329001- | GVPSRFSGSGSGTGFTLTISSLQPEDIATYYCQQAHSYPFTFGGGTKLEIKPDIQN |
| | T4.hCA | PDPAVYQLRDSKSSDKSVCLFTDFDSQTQVSQSKDSDVYITDKCVLDMRSMDFKSN |
| | (SEQ ID NO: | SAVAWSQKSDFACANAFQNSIIPEDTFFPSPESS |
| | 78) | |

While the disclosure has been particularly shown and described with reference to specific embodiments, it should be understood by those having skill in the art that various changes in form and detail may be made therein without departing from the spirit and scope of the present disclosure as disclosed herein.

1-36. (canceled)

37. A polypeptide complex comprising a first antigen-binding moiety and a second antigen-binding moiety, wherein:
the first antigen-binding moiety comprises
a first polypeptide comprising, from N-terminus to C-terminus, a first heavy chain variable domain (VH) of a first antibody operably linked to a first T cell receptor (TCR) constant region (C1), and
a second polypeptide comprising, from N-terminus to C-terminus, a first light chain variable domain (VL) of the first antibody operably linked to a second TCR constant region (C2),
wherein:
C1 comprises an engineered CBeta and C2 comprises an engineered CAlpha, or C1 comprises an engineered CAlpha and C2 comprises an engineered CBeta,
C1 and C2 are capable of forming a dimer comprising at least one non-native interchain bond between C1 and C2, and the non-native interchain bond is capable of stabilizing the polypeptide complex,
the engineered CAlpha and/or engineered CBeta further comprises at least one mutation at the c-terminus region of the engineered CAlpha and/or engineered CBeta, or at one or more amino acid positions that are spatially close to the c-terminus of engineered CAlpha and/or engineered CBeta, wherein the at least one mutation improves the stability of engineered CAlpha, the stability of engineered CBeta, and/or the interfacial stability of the engineered CAlpha-CBeta,
and
the first antibody has a first antigenic specificity; and
the second antigen-binding moiety has a second antigenic specificity.

38. The polypeptide complex of claim 37, wherein the engineered CAlpha comprises at least one mutated residue selected from P92S, E93D, S94V, and S95P, and/or the engineered CBeta comprises at least one mutated residue selected from E17K and S21A.

39. The polypeptide complex of claim 37, wherein the engineered CAlpha and/or CBeta comprise one or more mutated residues to form one or more non-native disulfide bonds, selected from: P8C on CAlpha, A9C on CAlpha, V10C on CAlpha, F26C on CAlpha, F29C on CAlpha, T33C on CAlpha, Q34C on CAlpha, V35C on CAlpha, S36C on CAlpha, S38C on CAlpha, K39C on CAlpha, F78C on CAlpha, N80C on CAlpha, S81C on CAlpha, I82C on CAlpha, P84C on CAlpha, D86C on CAlpha, T87C on CAlpha, F88C on CAlpha, F89C on CAlpha, P90C on CAlpha, and A18C on CBeta.

40. The polypeptide complex of claim 37, wherein the engineered CAlpha comprises at its C-terminus:
(a) a deletion of “FFPSPESS” (SEQ ID NO: 9) or “PESS” (SEQ ID NO: 6);
(b) mutations with “VEPKS” (SEQ ID NO: 5) in place of “PESS” (SEQ ID NO: 6); or
(c) mutations with “NRGE” (SEQ ID NO: 7) in place of “PESS” (SEQ ID NO: 6).

41. The polypeptide complex of claim 37, wherein the engineered CAlpha and the engineered CBeta comprise mutations S22F, T33I, and A73T on CAlpha and E17K, H22R, D38P, and S53D on CBeta.

42. The polypeptide complex of claim 37, wherein:
(a) the engineered CAlpha comprises mutated residues P8C and D86C on CAlpha, and a deletion of 4 amino acid residues at the C terminus of CAlpha (amino acid residues 92-95); or
(b) the engineered CAlpha and the engineered CBeta comprise mutated residues P90C on CAlpha and A18C on CBeta, and a deletion of 4 amino acid residues at the C terminus of CAlpha (amino acid residues 92-95).

43. The polypeptide complex of claim 37, wherein:
(a) the engineered C2 comprises any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 79, and 80, and/or the engineered C1 comprises any one of SEQ ID NOs: 11, 13, 15, 17, 19, 21, 23, 25, 27, 29, 31, 33, 35, 37, 39, 41, 43, 45, 47, 49, 51, 53, 55, 57, 59, 61, 63, and 65; or
(b) the engineered C1 comprises any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 79, and 80, and/or the engineered C2 comprises any one of SEQ ID NOs: 11, 13, 15, 17, 19, 21, 23, 25, 27, 29, 31, 33, 35, 37, 39, 41, 43, 45, 47, 49, 51, 53, 55, 57, 59, 61, 63, and 65.

44. The polypeptide complex of claim 37, wherein the engineered C1 and the engineered C2 respectively comprise a pair of sequences selected from the group consisting of SEQ ID NOs: 10/11, 12/13, 14/15, 16/17, 18/19, 20/21, 22/23, 24/25, 26/27, 28/29, 30/31, 32/33, 34/35, 36/37, 38/39, 40/41, 42/43, 44/45, 46/47, 48/49, 50/51, 52/53, 54/55, 56/57, 58/59, 60/61, 62/63, 64/65, 79/43 and 80/51; or
the engineered C2 and the engineered C1 respectively comprise a pair of sequences selected from the group consisting of SEQ ID NOs: 12/13, 14/15, 16/17, 18/19, 20/21, 22/23, 24/25, 26/27, 28/29, 30/31, 32/33, 34/35, 36/37, 38/39, 40/41, 42/43, 44/45, 46/47, 48/49, 50/51, 52/53, 54/55, 56/57, 58/59, 60/61, 62/63, 64/65, 79/43, and 80/51.

45. The polypeptide complex of claim 37, wherein:
(a) the engineered C2 and the engineered C1 respectively comprise a pair of sequences of SEQ ID NOs: 42 and 43, or the engineered C1 and the engineered C2 respectively comprise a pair of sequences of SEQ ID NOs: 42 and 43;
(b) the engineered C2 and the engineered C1 respectively comprise a pair of sequences of SEQ ID NOs: 50 and 51, or the engineered C1 and the engineered C2 respectively comprise a pair of sequences of SEQ ID NOs: 50 and 51;
(c) the engineered C2 and the engineered C1 respectively comprise a pair of sequences of SEQ ID NOs: 79 and 43, or the engineered C1 and the engineered C2 respectively comprise a pair of sequences of SEQ ID NOs: 79 and 43; or
(d) the engineered C2 and the engineered C1 respectively comprise a pair of sequences of SEQ ID NOs: 80 and 51, or the engineered C1 and the engineered C2 respectively comprise a pair of sequences of SEQ ID NOs: 80 and 51.

46. The polypeptide complex of claim 37, wherein one of the first and the second antigenic specificities is directed to an exogenous antigen, an endogenous antigen, an autoantigen, a neoantigen, a viral antigen or a tumor antigen.

47. The polypeptide complex of claim 37, wherein one of the first and the second antigenic specificities is directed to a T-cell specific receptor molecule and/or a natural killer cell (NK cell) specific receptor molecule, and the other is directed to a tumor associated antigen.

48. The polypeptide complex of claim 37, wherein:
(a) one of the first and the second antigenic specificities is directed to PD-L1, and the other is directed to 4-1BB;
(b) one of the first and the second antigenic specificities is directed to HER2 D2, and the other is directed to HER2 D4;
(c) one of the first and the second antigenic specificities is directed to IL-17, and the other is directed to IL-20; or
(d) one of the first and the second antigenic specificities is directed to IL-4, and the other is directed to IL-13.

49. A conjugate comprising the polypeptide complex of claim 37, conjugated to a moiety.

50. An isolated polynucleotide encoding the polypeptide complex of claim 37.

51. An isolated vector comprising the polynucleotide of claim 50.

52. A host cell comprising the isolated polynucleotide of claim 50.

53. A method of expressing the polypeptide complex of claim 37, comprising culturing a host cell under the condition at which the polypeptide complex is expressed, wherein the host cell comprises a polynucleotide encoding the polypeptide complex.

54. A composition comprising the polypeptide complex of claim 37, optionally wherein the composition is a pharmaceutical composition comprising the polypeptide complex and a pharmaceutically acceptable carrier.

55. A method of treating a condition in a subject in need thereof, comprising administrating to the subject a therapeutically effective amount of the polypeptide complex of claim 37, wherein the condition can be alleviated, eliminated, treated, or prevented when the first antigen and the second antigen are both modulated.

56. A kit comprising one or more containers comprising the polypeptide complex of claim 37.


SEQUENCE LISTING (82 sequences)

SEQ ID NO: 1 - protein, 94 aa, Artificial Sequence
  TRAC_Human CAlpha
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 2 - protein, 131 aa, Artificial Sequence
  TRBC2_Human CBeta
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCG

SEQ ID NO: 3 - protein, 10 aa, Artificial Sequence
  hinge region sequence
ERKCCERKSC

SEQ ID NO: 4 - protein, 4 aa, Artificial Sequence
  C-terminus amino acid sequence
EPKS

SEQ ID NO: 5 - protein, 5 aa, Artificial Sequence
  C-terminus amino acid sequence
VEPKS

SEQ ID NO: 6 - protein, 4 aa, Artificial Sequence
  C-terminus amino acid sequence
PESS

SEQ ID NO: 7 - protein, 4 aa, Artificial Sequence
  C-terminus amino acid sequence
NRGE

SEQ ID NO: 8 - protein, 127 aa, Artificial Sequence
  engineered CBeta
DLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGR

SEQ ID NO: 9 - protein, 8 aa, Artificial Sequence
  C-terminus amino acid sequence
FFPSPESS

SEQ ID NO: 10 - protein, 95 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 11 - protein, 141 aa, Artificial Sequence
  TCR
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCP

SEQ ID NO: 12 - protein, 96 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFPSSDVPC

SEQ ID NO: 13 - protein, 142 aa, Artificial Sequence
  TCR
LEDLKNVFPPEVAVFEPSKAEIAHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADCDKTHTCPPCP

SEQ ID NO: 14 - protein, 97 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFPSVEPKSC

SEQ ID NO: 15 - protein, 143 aa, Artificial Sequence
  TCR
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADCGDKTHTCPPCP

SEQ ID NO: 16 - protein, 96 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFPSNRGEC

SEQ ID NO: 17 - protein, 143 aa, Artificial Sequence; identical to SEQ ID NO: 15
  TCR

SEQ ID NO: 18 - protein, 87 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 19 - protein, 141 aa, Artificial Sequence; identical to SEQ ID NO: 11
  TCR

SEQ ID NO: 20 - protein, 91 aa, Artificial Sequence
  TCR
PDIQNPDPAVYQLRDSKSSDKSVCLCTDFDSQTQVSQSKDSDVYITDKCVLDMRSMDFKSNSAVAWSQKSDFACANACQNSIIPEDTFFPS

SEQ ID NO: 21 - protein, 141 aa, Artificial Sequence; identical to SEQ ID NO: 11
  TCR

SEQ ID NO: 22 - protein, 91 aa, Artificial Sequence
  TCR
PDIQNPDPAVYQLRDSKSSDKSVCLFTDFDSQTQVCQSKDSDVYITDKCVLDMRSMDFKSNSAVAWSQKSDFACANAFQCSIIPEDTFFPS

SEQ ID NO: 23 - protein, 141 aa, Artificial Sequence; identical to SEQ ID NO: 11
  TCR

SEQ ID NO: 24 - protein, 91 aa, Artificial Sequence
  TCR
PDIQNPDPAVYQLRDSKSSDKSVCLFTDFDSQTQVSQCKDSDVYITDKCVLDMRSMDFKSNSAVAWSQKSDFACANAFQCSIIPEDTFFPS

SEQ ID NO: 25 - protein, 141 aa, Artificial Sequence; identical to SEQ ID NO: 11
  TCR

SEQ ID NO: 26 - protein, 91 aa, Artificial Sequence
  TCR
PDIQNPDPAVYQLRDSKSSDKSVCLFTDFDSQTQVSQSCDSDVYITDKCVLDMRSMDFKSNSAVAWSQKSDFACANAFQCSIIPEDTFFPS

SEQ ID NO: 27 - protein, 141 aa, Artificial Sequence; identical to SEQ ID NO: 11
  TCR

SEQ ID NO: 28 - protein, 91 aa, Artificial Sequence
  TCR
PDIQNPDPAVYQLRDSKSSDKSVCLFTDFDSQTCVSQSKDSDVYITDKCVLDMRSMDFKSNSAVAWSQKSDFACANAFQNCIIPEDTFFPS

SEQ ID NO: 29 - protein, 141 aa, Artificial Sequence; identical to SEQ ID NO: 11
  TCR

SEQ ID NO: 30 - protein, 91 aa, Artificial Sequence
  TCR
PDIQNPDPAVYQLRDSKSSDKSVCLFTDFDSQTQCSQSKDSDVYITDKCVLDMRSMDFKSNSAVAWSQKSDFACANAFQNCIIPEDTFFPS

SEQ ID NO: 31 - protein, 141 aa, Artificial Sequence; identical to SEQ ID NO: 11
  TCR

SEQ ID NO: 32 - protein, 91 aa, Artificial Sequence
  TCR
PDIQNPDPAVYQLRDSKSSDKSVCLFTDFDSQTQVCQSKDSDVYITDKCVLDMRSMDFKSNSAVAWSQKSDFACANAFQNCIIPEDTFFPS

SEQ ID NO: 33 - protein, 141 aa, Artificial Sequence; identical to SEQ ID NO: 11
  TCR

SEQ ID NO: 34 - protein, 91 aa, Artificial Sequence
  TCR
PDIQNPDPAVYQLRDSKSSDKSVCLFTDFDSQTCVSQSKDSDVYITDKCVLDMRSMDFKSNSAVAWSQKSDFACANAFQNSCIPEDTFFPS

SEQ ID NO: 35 - protein, 141 aa, Artificial Sequence; identical to SEQ ID NO: 11
  TCR

SEQ ID NO: 36 - protein, 91 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIICEDTFFPS

SEQ ID NO: 37 - protein, 142 aa, Artificial Sequence
  TCR
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCTP

SEQ ID NO: 38 - protein, 91 aa, Artificial Sequence
  TCR
PDIQNPDPAVYQLRDSKSSDKSVCLFTDCDSQTQVSQSKDSDVYITDKCVLDMRSMDFKSNSAVAWSQKSDFACANAFQNSIICEDTFFPS

SEQ ID NO: 39 - protein, 141 aa, Artificial Sequence; identical to SEQ ID NO: 11
  TCR

SEQ ID NO: 40 - protein, 91 aa, Artificial Sequence
  TCR
PDIQNPDPAVYQLRDSKSSDKSVCLFTDFDSQCQVSQSKDSDVYITDKCVLDMRSMDFKSNSAVAWSQKSDFACANAFQNSIICEDTFFPS

SEQ ID NO: 41 - protein, 141 aa, Artificial Sequence; identical to SEQ ID NO: 11
  TCR

SEQ ID NO: 42 - protein, 91 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPECTFFPS

SEQ ID NO: 43 - protein, 141 aa, Artificial Sequence; identical to SEQ ID NO: 11
  TCR

SEQ ID NO: 44 - protein, 91 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPEDCFFPS

SEQ ID NO: 45 - protein, 141 aa, Artificial Sequence; identical to SEQ ID NO: 11
  TCR

SEQ ID NO: 46 - protein, 91 aa, Artificial Sequence
  TCR
PDIQNPDPCVYQLRDSKSSDKSVCLFTDFDSQTQVSQSKDSDVYITDKCVLDMRSMDFKSNSAVAWSQKSDFACANAFQNSIIPEDTCFPS

SEQ ID NO: 47 - protein, 141 aa, Artificial Sequence; identical to SEQ ID NO: 11
  TCR

SEQ ID NO: 48 - protein, 91 aa, Artificial Sequence
  TCR
PDIQNPDPACYQLRDSKSSDKSVCLFTDFDSQTQVSQSKDSDVYITDKCVLDMRSMDFKSNSAVAWSQKSDFACANAFQNSIIPEDTFCPS

SEQ ID NO: 49 - protein, 141 aa, Artificial Sequence; identical to SEQ ID NO: 11
  TCR

SEQ ID NO: 50 - protein, 91 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFCS

SEQ ID NO: 51 - protein, 141 aa, Artificial Sequence
  TCR
LEDLKNVFPPEVAVFEPSECEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCP

SEQ ID NO: 52 - protein, 91 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFPS

SEQ ID NO: 53 - protein, 141 aa, Artificial Sequence; identical to SEQ ID NO: 11
  TCR

SEQ ID NO: 54 - protein, 95 aa, Artificial Sequence
  TCR
PDIQNPDPAVYQLRDSKSSDKFVCLFTDFDSQIQVSQSKDSDVYITDKCVLDMRSMDFKSNSAVAWSQKSDFTCANAFQNSIIPEDTFFPSPESS

SEQ ID NO: 55 - protein, 141 aa, Artificial Sequence
  TCR
LEDLKNVFPPEVAVFEPSKAEISRTQKATLVCLATGFYPPHVELSWWVNGKEVHDGVCTDPQPLKEQPALQDSRYALSSRLRVSATFWQNPRNHFRCQVQFYGLSENDEWTQDRAKPVTQIVSAEAWGRASDKTHTCPPCP

SEQ ID NO: 56 - protein, 96 aa, Artificial Sequence
  TCR
PDIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSC

SEQ ID NO: 57 - protein, 142 aa, Artificial Sequence
  TCR
LEDLKNVFPPEVAVFEPSKAEISRTQKATLVCLATGFYPPHVELSWWVNGKEVHDGVCTDPQPLKEQPALQDSRYALSSRLRVSATFWQNPRNHFRCQVQFYGLSENDEWTQDRAKPVTQIVSAEAWGRADCDKTHTCPPCP

SEQ ID NO: 58 - protein, 91 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPECTFFCS

SEQ ID NO: 59 - protein, 141 aa, Artificial Sequence; identical to SEQ ID NO: 51
  TCR

SEQ ID NO: 60 - protein, 96 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPECTFFPSPESSC

SEQ ID NO: 61 - protein, 142 aa, Artificial Sequence
  TCR
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADCDKTHTCPPCP

SEQ ID NO: 62 - protein, 96 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFCSPESSC

SEQ ID NO: 63 - protein, 142 aa, Artificial Sequence
  TCR
LEDLKNVFPPEVAVFEPSECEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADCDKTHTCPPCP

SEQ ID NO: 64 - protein, 96 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPECTFFCSPESSC

SEQ ID NO: 65 - protein, 142 aa, Artificial Sequence; identical to SEQ ID NO: 63
  TCR

SEQ ID NO: 66 - protein, 708 aa, Artificial Sequence
  chain 1
QVQLQESGPGLVKPSETLSLTCTVSGFSLTENSVSWIRQPPGKGLEWIGAVWSSGSTDYNSALKSRVTISRDTSKNQFSLKLSSVTAADTAVYYCTRSTYSNDFYYYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSQEQLQESGPGLVKPSQTLSLTCTVSGGSINSQGYYWSWIRQHPGKGLEWIGYIYDSGSAYYNPSLERRVAISLDTSKNQFSLNLNSVTVADTAVYYCARIVAAGRIDPWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 214 aa, Artificial Sequence
  chain 2
SYELTQPPSVSVSPGQTASITCSGSELPKRYAYWYQQKPGQSIVRVIYKDSERPSGISERFSGSSSGNTATLTISGTQAMDEADYYCSSTYGDRKLPIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 68 - protein, 203 aa, Artificial Sequence
  chain 3
SYVLTQPPSVSVAPGQTARMTCGGDNIGIKIVHWYQQKAGQAPVLVVYDDNDRPSGIPDRFSGSNSGNTATLTISRVAAGDEADYYCQVWDRRSDHVVFGGGTKLTVLPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 69 - protein, 448 aa, Artificial Sequence
  chain 1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 475 aa, Artificial Sequence
  chain 2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 71 - protein, 214 aa, Artificial Sequence
  chain 3
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 202 aa, Artificial Sequence
  chain 4
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 73 - protein, 713 aa, Artificial Sequence
  chain 1
QVQLVQSGAEVKRPGASVKVSCKASGYTFTNDIIHWVRQAPGQRLEWMGWINAGYGNTQYSQNFQDRVSITRDTSASTAYMELISLRSEDTAVYYCAREPLWFGESSPHDYYGMDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSQVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 214 aa, Artificial Sequence
  chain 2
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 207 aa, Artificial Sequence
  chain 3
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 76 - protein, 709 aa, Artificial Sequence
  chain 1
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSAVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 218 aa, Artificial Sequence
  chain 2
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 202 aa, Artificial Sequence
  chain 3
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 79 - protein, 95 aa, Artificial Sequence
  TCR
PDIQNPDCAVYQLRDSKSSDKSVCLFTDFDSQTQVSQSKDSDVYITDKCVLDMRSMDFKSNSAVAWSQKSDFACANAFQNSIIPECTFFPSPESS

SEQ ID NO: 80 - protein, 95 aa, Artificial Sequence
  TCR
PDIQNPDPAVYQLRDSKSSDKSVCLFTDFDSQTQVSQSKDSDVYITDKCVLDMRSMDFKSNSAVAWSQKSDFACANAFQNSIIPEDTFFCSPESS

SEQ ID NO: 81 - protein, 4 aa, Artificial Sequence
  Synthetic peptide
YPSN

SEQ ID NO: 82 - protein, 4 aa, Artificial Sequence
  Synthetic peptide
QSGR